SEQUENCE LISTING (25 sequences)

SEQ ID NO: 1 - DNA, 644 bp, synthetic construct
  source 1..644: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 2 - DNA, 120 bp, synthetic construct
  source 1..120: mol_type = other DNA
gtaagagcctagcatgtagaactggttacctgcagcccaagcttgctgcacgtctagggcctcaccgggtttccttgatgaggtaccgacatacttatcctgtcccttttttttccacag

SEQ ID NO: 3 - DNA, 941 bp, synthetic construct
  source 1..941: mol_type = other DNA
gtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcggggagtcacccacacaaaggaaaagggcctttccgtcctcagccgtcgcttcatgtgactccacggagtaccgggcgccgtccaggcacctcgattagttctcgagcttttggatacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 4 - DNA, 321 bp, synthetic construct
  source 1..321: mol_type = other DNA
tgtttgaatgaggcttcagtactttacagaatcgttgcctgcacatcttggaaacacttgctgggattacttcgacttcttaacccaacagaaggctcgagaaggtatattgctgttgacagtgagcgaaacgatatgggctgaatacaatagtgaagccacagatgtattgtattcagcccatatcgttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 5 - DNA, 321 bp, synthetic construct
  source 1..321: mol_type = other DNA
tgtttgaatgaggcttcagtactttacagaatcgttgcctgcacatcttggaaacacttgctgggattacttcgacttcttaacccaacagaaggctcgagaaggtatattgctgttgacagtgagcgaaagcgccaaaggagtctgtgatagtgaagccacagatgtatcacagactcctttggcgcttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 6 - DNA, 321 bp, synthetic construct
  source 1..321: mol_type = other DNA
tgtttgaatgaggcttcagtactttacagaatcgttgcctgcacatcttggaaacacttgctgggattacttcgacttcttaacccaacagaaggctcgagaaggtatattgctgttgacagtgagcgaaagtgcggatttcgtatttgctagtgaagccacagatgtagcaaatacgaaatccgcacttgtgcctactgcctcggacttcaaggggctagaattcgagcaattatcttgtttactaaaactgaataccttgctatctctttgatacatttttacaaagctgaattaaaatggtataaattaaatcacttt

SEQ ID NO: 7 - DNA, 1497 bp, synthetic construct
  source 1..1497: mol_type = other DNA
atggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctga

SEQ ID NO: 8 - DNA, 105 bp, synthetic construct
  source 1..105: mol_type = other DNA
gctatgaaacgatatgggctgaatacaaatcacaggctatgaaacgatatgggctgaatacaaatcacaggctatgaaacgatatgggctgaatacaaatcacag

SEQ ID NO: 9 - DNA, 105 bp, synthetic construct
  source 1..105: mol_type = other DNA
gctatgaaagcgccaaaggagtctgtgaatcacaggctatgaaagcgccaaaggagtctgtgaatcacaggctatgaaagcgccaaaggagtctgtgaatcacag

SEQ ID NO: 10 - DNA, 105 bp, synthetic construct
  source 1..105: mol_type = other DNA
gctatgaaagtgcggatttcgtatttgcatcacaggctatgaaagtgcggatttcgtatttgcatcacaggctatgaaagtgcggatttcgtatttgcatcacag

SEQ ID NO: 11 - DNA, 247 bp, synthetic construct
  source 1..247: mol_type = other DNA
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggt

SEQ ID NO: 12 - DNA, 131 bp, synthetic construct
  source 1..131: mol_type = other DNA
acttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 13 - DNA, 10 bp, synthetic construct
  source 1..10: mol_type = other DNA
gccaccatgg

SEQ ID NO: 14 - DNA, 8328 bp, synthetic construct
  source 1..8328: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagcggactgtttgaatgaggcttcagtactttacagaatcgttgcctgcacatcttggaaacacttgctgggattacttcgacttcttaacccaacagaaggctcgagaaggtatattgctgttgacagtgagcgaaacgatatgggctgaatacaatagtgaagccacagatgtattgtattcagcccatatcgttgtgcctactgcctcggacttcaaggggctagaattcgagcaattatcttgtttactaaaactgaataccttgctatctctttgatacatttttacaaagctgaattaaaatggtataaattaaatcactttcactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcggggagtcacccacacaaaggaaaagggcctttccgtcctcagccgtcgcttcatgtgactccacggagtaccgggcgccgtccaggcacctcgattagttctcgagcttttggatacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcgccaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgaggatcctaatgctgctatgaaacgatatgggctgaatacaaatcacaggctatgaaacgatatgggctgaatacaaatcacaggctatgaaacgatatgggctgaatacaaatcacagggtagctagcgagctcacttgtttattgcagcttataatggttacaaataaagcaatagcatcacaaatttcacaaataaagcatttttttcactgcattctagttgtggtttgtccaaactcatcaatgtatcttatcatgtctgttccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagctt

SEQ ID NO: 15 - DNA, 8575 bp, synthetic construct
  source 1..8575: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagcggactgtttgaatgaggcttcagtactttacagaatcgttgcctgcacatcttggaaacacttgctgggattacttcgacttcttaacccaacagaaggctcgagaaggtatattgctgttgacagtgagcgaaacgatatgggctgaatacaatagtgaagccacagatgtattgtattcagcccatatcgttgtgcctactgcctcggacttcaaggggctagaattcgagcaattatcttgtttactaaaactgaataccttgctatctctttgatacatttttacaaagctgaattaaaatggtataaattaaatcactttcactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcggggagtcacccacacaaaggaaaagggcctttccgtcctcagccgtcgcttcatgtgactccacggagtaccgggcgccgtccaggcacctcgattagttctcgagcttttggatacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcgccaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgaggatcctaatgctgctatgaaacgatatgggctgaatacaaatcacaggctatgaaacgatatgggctgaatacaaatcacaggctatgaaacgatatgggctgaatacaaatcacagggtagctagcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgagctcacttgtttattgcagcttataatggttacaaataaagcaatagcatcacaaatttcacaaataaagcatttttttcactgcattctagttgtggtttgtccaaactcatcaatgtatcttatcatgtctgttccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagctt

SEQ ID NO: 16 - DNA, 7868 bp, synthetic construct
  source 1..7868: mol_type = other DNA
ccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacattattgaagcatttatcagggttattgtctcagacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggattcttctgacacaacagtctcgaacttaagctgcagaagttggtcgtgaggcactgggcaggtgtccactcccagttcaattacagctcttaaggctagagtactcggaaacccgtcggcctccgaacggtaagagcctagcatgtagaactggttacctgcagcccaagcttgctgcacgtctagggcggactgtttgaatgaggcttcagtactttacagaatcgttgcctgcacatcttggaaacacttgctgggattacttcgacttcttaacccaacagaaggctcgagaaggtatattgctgttgacagtgagcgaaacgatatgggctgaatacaatagtgaagccacagatgtattgtattcagcccatatcgttgtgcctactgcctcggacttcaaggggctagaattcgagcaattatcttgtttactaaaactgaataccttgctatctctttgatacatttttacaaagctgaattaaaatggtataaattaaatcactttcagctcaccgggtttccttgatgaggtaccgacatacttatcctgtcccttttttttccacagctcgcggttgaggacaaactcttcaccggtcgccaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgaggatcctaatgctgctatgaaacgatatgggctgaatacaaatcacaggctatgaaacgatatgggctgaatacaaatcacaggctatgaaacgatatgggctgaatacaaatcacagggtagctagcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgagctcacttgtttattgcagcttataatggttacaaataaagcaatagcatcacaaatttcacaaataaagcatttttttcactgcattctagttgtggtttgtccaaactcatcaatgtatcttatcatgtctgttccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 17 - DNA, 8575 bp, synthetic construct
  source 1..8575: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagcggactgtttgaatgaggcttcagtactttacagaatcgttgcctgcacatcttggaaacacttgctgggattacttcgacttcttaacccaacagaaggctcgagaaggtatattgctgttgacagtgagcgaaacgatatgggctgaatacaatagtgaagccacagatgtattgtattcagcccatatcgttgtgcctactgcctcggacttcaaggggctagaattcgagcaattatcttgtttactaaaactgaataccttgctatctctttgatacatttttacaaagctgaattaaaatggtataaattaaatcactttcactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcggggagtcacccacacaaaggaaaagggcctttccgtcctcagccgtcgcttcatgtgactccacggagtaccgggcgccgtccaggcacctcgattagttctcgagcttttggatacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcgccaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgaggatcctaatgctgctatgaaacgatatgggctcaatacaaatcacaggctatgaaacgatatgggctcaatacaaatcacaggctatgaaacgatatgggctcaatacaaatcacagggtagctagcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgagctcacttgtttattgcagcttataatggttacaaataaagcaatagcatcacaaatttcacaaataaagcatttttttcactgcattctagttgtggtttgtccaaactcatcaatgtatcttatcatgtctgttccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagctt

SEQ ID NO: 18 - DNA, 8575 bp, synthetic construct
  source 1..8575: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagcggactgtttgaatgaggcttcagtactttacagaatcgttgcctgcacatcttggaaacacttgctgggattacttcgacttcttaacccaacagaaggctcgagaaggtatattgctgttgacagtgagcgaaacgatatgggctgaatacaatagtgaagccacagatgtattgtattcagcccatatcgttgtgcctactgcctcggacttcaaggggctagaattcgagcaattatcttgtttactaaaactgaataccttgctatctctttgatacatttttacaaagctgaattaaaatggtataaattaaatcactttcactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcggggagtcacccacacaaaggaaaagggcctttccgtcctcagccgtcgcttcatgtgactccacggagtaccgggcgccgtccaggcacctcgattagttctcgagcttttggatacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcgccaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgaggatcctaatgctgctatgaaacgatatgggctgaatactaatcacaggctatgaaacgatatgggctgaatactaatcacaggctatgaaacgatatgggctgaatactaatcacagggtagctagcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgagctcacttgtttattgcagcttataatggttacaaataaagcaatagcatcacaaatttcacaaataaagcatttttttcactgcattctagttgtggtttgtccaaactcatcaatgtatcttatcatgtctgttccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagctt

SEQ ID NO: 19 - DNA, 8575 bp, synthetic construct
  source 1..8575: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagcggactgtttgaatgaggcttcagtactttacagaatcgttgcctgcacatcttggaaacacttgctgggattacttcgacttcttaacccaacagaaggctcgagaaggtatattgctgttgacagtgagcgaaagcgccaaaggagtctgtgatagtgaagccacagatgtatcacagactcctttggcgcttgtgcctactgcctcggacttcaaggggctagaattcgagcaattatcttgtttactaaaactgaataccttgctatctctttgatacatttttacaaagctgaattaaaatggtataaattaaatcactttcactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcggggagtcacccacacaaaggaaaagggcctttccgtcctcagccgtcgcttcatgtgactccacggagtaccgggcgccgtccaggcacctcgattagttctcgagcttttggatacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcgccaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgaggatcctaatgctgctatgaaagcgccaaaggagtctgtgaatcacaggctatgaaagcgccaaaggagtctgtgaatcacaggctatgaaagcgccaaaggagtctgtgaatcacagggtagctagcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgagctcacttgtttattgcagcttataatggttacaaataaagcaatagcatcacaaatttcacaaataaagcatttttttcactgcattctagttgtggtttgtccaaactcatcaatgtatcttatcatgtctgttccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagctt

SEQ ID NO: 20 - DNA, 8575 bp, synthetic construct
  source 1..8575: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagcggactgtttgaatgaggcttcagtactttacagaatcgttgcctgcacatcttggaaacacttgctgggattacttcgacttcttaacccaacagaaggctcgagaaggtatattgctgttgacagtgagcgaaagtgcggatttcgtatttgctagtgaagccacagatgtagcaaatacgaaatccgcacttgtgcctactgcctcggacttcaaggggctagaattcgagcaattatcttgtttactaaaactgaataccttgctatctctttgatacatttttacaaagctgaattaaaatggtataaattaaatcactttcactggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcggggagtcacccacacaaaggaaaagggcctttccgtcctcagccgtcgcttcatgtgactccacggagtaccgggcgccgtccaggcacctcgattagttctcgagcttttggatacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcgccaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgaggatcctaatgctgctatgaaagtgcggatttcgtatttgcatcacaggctatgaaagtgcggatttcgtatttgcatcacaggctatgaaagtgcggatttcgtatttgcatcacagggtagctagcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgagctcacttgtttattgcagcttataatggttacaaataaagcaatagcatcacaaatttcacaaataaagcatttttttcactgcattctagttgtggtttgtccaaactcatcaatgtatcttatcatgtctgttccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacagctt

SEQ ID NO: 21 - DNA, 919 bp, synthetic construct
  source 1..919: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggattcttctgacacaacagtctcgaacttaagctgcagaagttggtcgtgaggcactgggcaggtgtccactcccagttcaattacagctcttaaggctagagtactcggaaacccgtcggcctccgaacggtaagagcctagcatgtagaactggttacctgcagcccaagcttgctgcacgtctagggcctcaccgggtttccttgatgaggtaccgacatacttatcctgtcccttttttttccacagctcgcggttgaggacaaactcttc

SEQ ID NO: 22 - DNA, 1629 bp, synthetic construct
  source 1..1629: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 23 - DNA, 222 bp, synthetic construct
  source 1..222: mol_type = other DNA
tctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggac

SEQ ID NO: 24 - DNA, 75 bp, synthetic construct
  source 1..75: mol_type = other DNA
aagaaagccgtgaaggagtcttctatccgatctgtgcaggagaccgtactccccatcaagaagcgcaagacccgg

SEQ ID NO: 25 - DNA, 3594 bp, synthetic construct
  source 1..3594: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggattcttctgacacaacagtctcgaacttaagctgcagaagttggtcgtgaggcactgggcaggtgtccactcccagttcaattacagctcttaaggctagagtactcggaaacccgtcggcctccgaacggtaagagcctagcatgtagaactggttacctgcagcccaagcttgctgcacgtctagggcggactgtttgaatgaggcttcagtactttacagaatcgttgcctgcacatcttggaaacacttgctgggattacttcgacttcttaacccaacagaaggctcgagaaggtatattgctgttgacagtgagcgaaacgatatgggctgaatacaatagtgaagccacagatgtattgtattcagcccatatcgttgtgcctactgcctcggacttcaaggggctagaattcgagcaattatcttgtttactaaaactgaataccttgctatctctttgatacatttttacaaagctgaattaaaatggtataaattaaatcactttcagctcaccgggtttccttgatgaggtaccgacatacttatcctgtcccttttttttccacagctcgcggttgaggacaaactcttcaccggtcgccaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgaggatcctaatgctgctatgaaacgatatgggctgaatacaaatcacaggctatgaaacgatatgggctgaatacaaatcacaggctatgaaacgatatgggctgaatacaaatcacagggtagctagcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgagctcacttgtttattgcagcttataatggttacaaataaagcaatagcatcacaaatttcacaaataaagcatttttttcactgcattctagttgtggtttgtccaaactcatcaatgtatcttatcatgtctgttccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

(19) United States
(12) Patent Application Publication
(10) Pub. No.: US 2025/0136651 A1
(43) Pub. Date: May 1, 2025

(54) RECOMBINANT OPTIMIZED MECP2 CASSETTES AND METHODS FOR TREATING RETT SYNDROME AND RELATED DISORDERS

(71) Applicant: THE UNIVERSITY COURT OF THE UNIVERSITY OF EDINBURGH, Edinburgh, Lothian (GB)

(72) Inventors: Stuart Robert Cobb, Edinburgh, Lothian (GB); Paul Ross, Edinburgh, Lothian (GB); Ralph David Hector, Edinburgh, Lothian (GB)

(21) Appl. No.: 18/832,458

(22) PCT Filed: Jan. 31, 2023
(86) PCT No.: PCT/GB2023/050207

§ 371 Date: Jul. 23, 2024

(30) Foreign Application Priority Data
Jan. 31, 2022 (GB) 2201242.1

(51) Int. Cl. C07K 14/47; A61K 38/17; A61K 48/00; C12N 15/113; C12N 15/86

(52) U.S. Cl. CPC C07K 14/4702; A61K 38/1709; A61K 48/005; C12N 15/113; C12N 15/86; C12N 2310/141; C12N 2750/14143; C12N 2830/48; C12N 2830/50

(57) ABSTRACT

The disclosure provides for optimized therapeutic MECP2 polynucleotide constructs utilized for replacing or compensating for the loss of MeCP2 function in patients with Rett Syndrome. Suitably, the disclosure provides gene therapy cassettes to enable better regulatory control of the MeCP2 protein, including tunable systems that allow MECP2 gene therapy to be expressed at a desired moderate level and demonstrate expression of the vector-derived transgene within a window that alleviates the disease-causing genetic deficiency without producing undesired side effects including overexpression toxicity.

### CROSS-REFERENCE TO RELATED APPLICATIONS

This application claims the benefit of and priority to United Kingdom Patent Application No. GB2201242.1, filed on Jan. 31, 2022, which is hereby incorporated by reference in its entirety.

### SEQUENCE LISTING

The instant application contains a Sequence Listing with 25 sequences, which has been submitted electronically in XML format and is hereby incorporated herein by reference in its entirety. Said XML copy, created on Jan. 24, 2023 is named P265708.WO.01_sequencelisting.xml, and is 90 kbytes in size.

### BACKGROUND

DNA methylation is the major modification of eukaryotic genomes and plays an essential role in mammalian development. Human proteins MeCP2, MBD1, MBD2, MBD3, and MBD4 comprise a family of nuclear proteins related by the presence in each of a methyl-CpG binding domain (MBD). Each of these proteins, with the exception of MBD3, is capable of binding specifically to methylated DNA. MeCP2, MBD1 and MBD2 can also repress transcription from methylated gene promoters. In contrast to other MBD family members, MeCP2 (methyl CpG binding protein 2) is X-linked and subject to X inactivation. MeCP2 is dispensable in stem cells but is essential for embryonic development.

Rett syndrome (RTT) is a neurodevelopmental disorder caused by mutations in the methyl CpG binding protein 2 (MECP2) gene. There are currently no approved treatments for RTT. Rett syndrome, a progressive neurologic developmental disorder is one of the most common causes of cognitive disability in females. Alternative splicing results in multiple transcript variants encoding different isoforms.

Although gene therapy provides for the delivery of a therapeutic transgene to affect correction in a genetic disease, many genes such as MECP2 are highly dosage sensitive whereby too little or too much expression of a gene product can have deleterious effects. Viral-mediated gene transfer is a powerful means to deliver therapeutic transgenes to target tissues and cells including cells of the nervous system. However, high virus titers are typically necessary to enable effective system-wide transduction for maximal therapeutic impact. Consequently, these same high titers may cause overexpression toxicity due to supraphysiological levels of transgene expression achieved in some cells. A transgene system that provides for dosage control of therapeutic transgenes is described in WO/2022/003348. Additional therapeutic constructs that provide optimal expression levels and further control of MeCP2 that are suitable for treating Rett Syndrome are needed.

### SUMMARY

The disclosure provides for optimized therapeutic MECP2 polynucleotide constructs utilized for replacing or compensating for the loss of MeCP2 function in patients with Rett Syndrome. Suitably, the disclosure provides gene therapy cassettes to enable better regulatory control of the MeCP2 protein, including tunable systems that allow MECP2 gene therapy to be expressed at a desired moderate level, as shown in FIG. 5A-C. Suitably, the MECP2 gene therapy constructs may demonstrate efficacy and clear improvement in motor and breathing phenotype domains in a mouse model of RTT (FIG. 6A-B and FIG. 7A-F). Suitably, polynucleotide constructs including RTT252, RTT253, RTT254 (also referred to as NGN-401), RTT269, RTT270, RTT271 and RTT272 may demonstrate expression of the vector-derived transgene within a window that alleviates the disease-causing genetic deficiency without producing undesired side effects including overexpression toxicity (FIG. 3A-C and FIG. 4A-C).

Suitably provided herein is a polynucleotide comprising from 5′ to 3′:
a promoter;
at least one non-mammalian or synthetic miRNA expressed within an intron;
a protein translation initiation site (Kozak sequence);
a human MECP2 coding sequence of SEQ ID NO: 7, a nucleotide sequence having at least 90% identity to SEQ ID NO:7, a codon optimized or wildtype human MECP2 coding sequence;
at least one 3′ stability element;
at least three miRNA binding sites for the non-mammalian or synthetic miRNA;
optionally the miRNA binding site comprises at least one mismatch, optionally a single mismatch;
and a polyadenylation signal.

In certain aspects provided herein is a polynucleotide comprising from 5′ to 3′:
a promoter;
at least one non-mammalian or synthetic miRNA expressed within an intron;
a protein translation initiation site (Kozak sequence);
a codon optimized or wildtype human MECP2 coding sequence;
at least one 3′ stability element;
at least three miRNA binding sites for the non-mammalian or synthetic miRNA;
optionally the miRNA binding site comprises at least one mismatch, optionally a single mismatch, optionally three miRNA binding sites for the non-mammalian or synthetic miRNA; optionally wherein the miRNA binding site comprises a single mismatch;
and a polyadenylation signal.

Micro RNAs (miRNAs) are a class of small, single-stranded, non-coding RNAs of ˜22 nucleotides in length. Most miRNAs are transcribed by RNA polymerase II, either as independent transcripts or as RNAs embedded within introns of mRNAs. Primary miRNA transcripts are processed into ˜70 nt hairpin precursor miRNAs and then finally to ˜22 nt mature miRNAs by two RNase III enzymes (Drosha and Dicer). miRNAs function by regulating protein levels, targeting messenger RNAs (mRNAs) for translational repression and/or mRNA degradation. The inventors have developed non-mammalian or synthetic miRNAs that are capable of knocking-down expression of transcripts containing the respective binding region. In some instances, these are insect-derived miRNA sequences originally designed to target the firefly luciferase protein. In other instances, they are synthetic miRNA sequences, with no orthology to naturally occurring miRNAs. In some instances, synthetic miRNA sequences are designed to target codon optimized coding sequences, where the coding sequence is altered at the DNA level while retaining the same amino acid sequence. In a gene therapy context, this allows exogenously delivered transgenes to be exclusively targeted by the synthetic miRNAs, whilst endogenous genes are unaffected. Suitably a miRNA may be embedded within different introns.

Suitably, the polynucleotide may comprise one non-mammalian or synthetic miRNA expressed within an intron. Suitably, the non-mammalian or synthetic miRNA may comprise SEQ ID NO: 4. Suitably, the human MECP2 coding sequence may comprise a nucleotide sequence having at least 90% identity at least 95%, at least 97%, at least 99%, at least 100% identity to SEQ ID NO:7. Suitably, the MECP2 sequence may be a codon optimized human MECP2 sequence. Suitably, the protein translation initiation site may be a Kozak sequence comprising SEQ ID NO: 13. Suitably the polynucleotide may comprise, a human MECP2 coding sequence or any active, suitably functionally active similar to the complete sequence, fragment thereof, including a minigene encoding such a functional fragment, wherein the coding sequence comprises a nucleotide sequence having at least 90% identity to SEQ ID NO:7, or to SEQ ID NO: 23, which encodes the Methyl-CpG Binding Domain (MBD) of MeCP2, or SEQ ID NO: 24, which encodes the NCoR/SMRT Interaction Domain (NID) of MeCP2.

>SEQ ID NO: 23
TCTGCCTCCCCCAAACAGCGGCGCTCCATCATCCGTGACCGGGGACCCA
TGTATGATGACCCCACCCTGCCTGAAGGCTGGACACGGAAGCTTAAGCA
AAGGAAATCTGGCCGCTCTGCTGGGAAGTATGATGTGTATTTGATCAAT
CCCCAGGGAAAAGCCTTTCGCTCTAAAGTGGAGTTGATTGCGTACTTCG
AAAAGGTAGGCGACACATCCCTGGAC
>SEQ ID NO: 24
AAGAAAGCCGTGAAGGAGTCTTCTATCCGATCTGTGCAGGAGACCGTAC
TCCCCATCAAGAAGCGCAAGACCCGG

Suitably, the promoter may comprise CBM or CBE (SEQ ID NO:21 or 22).

Suitably, the CBM promoter may comprise a nucleotide sequence having at least 90% identity, at least 95%, at least 97%, at least 99%, at least 100% identity to SEQ ID NO:21. Suitably, the CBE promoter may comprise a nucleotide sequence having at least 90% identity at least 95%, at least 97%, at least 99%, at least 100% identity to SEQ ID NO:22. Suitably, the at least one 3′ stability element may be WPRE. Suitably, the polynucleotide comprises three miRNA binding sites for the non-mammalian or synthetic miRNA. Suitably, the miRNA binding sites may comprise SEQ ID NO: 8. Suitably, the miRNA binding site may comprise one mismatch. Suitably, the polyadenylation signal may be a simian vacuolating virus 40 polyadenylation signal (SV40 pA). Suitably, the SV40 pA signal comprises the nucleotide sequence of SEQ ID NO:12.

Suitably, the polynucleotide may comprise: a CBM promoter, one non-mammalian or synthetic miRNA expressed within an intron, a wild-type human MECP2 coding sequence with an optimized Kozak sequence, a WPRE stability element, three miRNA binding sites for the non-mammalian or synthetic miRNA, and an SV40 pA signal.

Suitably, the polynucleotide may comprise: a CBM promoter, one non-mammalian or synthetic miRNA expressed within an intron, a codon optimized human MECP2 coding sequence with an optimized Kozak sequence, a WPRE stability element, three miRNA binding sites for the non-mammalian or synthetic miRNA, and an SV40 pA signal.

Suitably, the polynucleotide construct may comprise SEQ ID NO: 25 (RTT254). Suitably, the polynucleotide construct may comprise a nucleotide sequence having at least 90% identity, at least 95%, at least 97% or at least 99% identity to SEQ ID NO:25.

Suitably, the polynucleotide may further comprise at least one adeno-associated virus (AAV) inverted terminal repeat (ITR).

Suitably, the polynucleotide may comprise two AAV ITRs.

Suitably, the disclosure provides a vector comprising the polynucleotide of any of the embodiments described herein.

Suitably, the vector may be a viral vector.

Suitably, the vector may be an adeno-associated virus (AAV) vector.

Suitably, the AAV vector may be an AAV9 vector.

In another aspect, the present disclosure provides a recombinant adeno-associated virus (rAAV), comprising any of the polynucleotides or vectors described herein. In certain embodiments, the rAAV is AAV9.

In another aspect, the present disclosure provides a virion comprising the rAAV described herein.

In another aspect, the present disclosure provides a transformed cell comprising any of the polynucleotides described herein, the vectors described herein, the rAAVs described herein, or the virions described herein.

In another aspect, the present disclosure provides a pharmaceutical composition comprising any of the polynucleotides described herein, the vectors described herein, the rAAVs described herein, or the virions described herein, and optionally, a pharmaceutically acceptable carrier.

In another aspect, the present disclosure provides a method of treating a MECP2-associated disorder in a subject, the method comprising administering to the subject an effective amount of any of the polynucleotides described herein, the vectors described herein, the rAAVs described herein, or the virions described herein, or the pharmaceutical compositions described herein.

Also provided is an effective amount of any of the polynucleotides described herein, the vectors described herein, the rAAVs described herein, or the virions described herein, or the pharmaceutical compositions described herein for use as a medicament, particularly for use in treating a MECP2-associated disorder in a subject.

Further provided is use of any of the polynucleotides described herein, the vectors described herein, the rAAVs described herein, or the virions described herein, or the pharmaceutical compositions described herein in the preparation of a medicament for the treatment of a MECP2-associated disorder in a subject

In another aspect, the treated subject exhibits improvement in one or more symptoms associated with a MECP2-associated disorder.

In another aspect, the subject is dosed with 1.0×10¹⁵ vg comprising NGN-401, delivered via a 10 mL ICV injection at 1.0×10¹⁴ vg/mL. Suitably, the subject is a human. Suitably, the subject is dosed at a range of 1.0×10¹⁴ to 1.0×10¹⁶ vg. Suitably, the subject is dosed at a range of 1.0×10¹⁴ to 1.0×10¹⁶ vg comprising NGN-401 (SEQ ID NO:25). Suitably, the dose is delivered via ICV injection, particularly via a 10 mL ICV injection. Suitably, the dose comprising NGN-401 is delivered via ICV injection, particularly via a 10 mL ICV injection. Suitably, the dose is delivered via a 10 mL ICV injection at a range of 1.0×10¹³ to 1.0×10¹⁵ vg/mL.

In another aspect, the effective dose is 8.3×10¹¹ vg/g brain. Suitably, the effective dose is 8.2×10¹¹ vg/g brain to 8.4×10¹¹ vg/g brain. Suitably, the effective dose is 8.3×10¹⁰ vg/g brain to 8.3×10¹² vg/g brain. Suitably, the subject is a human. Suitably, the polynucleotide construct may comprise SEQ ID NO: 25 (NGN-401/RTT254).

In another aspect, the subject is substantially free of MECP2 overexpression toxicity.

### BRIEF DESCRIPTION OF THE DRAWINGS

FIG. 1A is a graphic depiction illustrating the MeCP2 dosage sensitive gene therapy cassettes designed to reduce dosage sensitivity, prevent overexpression and achieve a therapeutic setpoint transgene level.

FIG. 1B shows graphs of flow cytometry data illustrating the effects of different modifications of the therapeutic cassette (feed forward circuit) for tuning MeCP2 protein expression level. Reporter constructs, in which the reporter mNeonGreen is fused to hMeCP2 and a second expression cassette allowing mRuby to be measured as a transfection control, were transfected into HEK cells and after 48 hrs cells were processed, analyzed by flow cytometry and levels of mRuby (transfection efficiency) and mNeonGreen (MeCP2) were measured.

FIG. 2 depicts a schematic showing the polynucleotide cassette elements that are modulated to adjust dosage insensitivity and setpoint of expression of MeCP2.

FIGS. 3A-C show a table depicting the modular polynucleotide sequence elements and design strategy for the MeCP2 constructs (FIG. 3A) along with MeCP2 expression data. 21-23 days after dosing wild-type mice with AAV9-RTT252, AAV9-RTT253, AAV9-RTT254, AAV9-RTT269, AAV9-RTT270, AAV9-RTT271 or AAV9-RTT272, tissue samples were collected and analyzed by western blot to determine levels of MeCP2 expression in WT cortex (FIG. 3B) and WT hippocampus (FIG. 3C).

FIGS. 4A-C are graphic depictions comparing the therapeutic MEPC2 constructs for survival (FIG. 4A), bodyweight (FIG. 4B), and RTT clinical score (FIG. 4C) in Mecp2^{-/y} (KO) mice following injection at P1 with 3×10¹¹ vg/mouse of a therapeutic AAV9-MECP2 construct. The RTT clinical score is an observational scoring system used to determine the severity of the Rett phenotype in mice. Scoring ranges from 0 (like wild-type) to 5 (most severe) for each individual component of the phenotype.

FIGS. 5A-C depict the systematic tuning using different polynucleotide cassette components to identify and titrate expression levels to obtain optimal efficacy-which is an intermediate or moderate level of expression. Survival plots and RTT clinical scores are shown for Mecp2^{-/y} animals dosed with 3×10¹¹ vg/mouse of an AAV9-MECP2 construct expressing weak (FIG. 5A), moderate (FIG. 5B) or strong (FIG. 5C) levels of transgenic MeCP2.

FIGS. 6A-B depict the improvement in survival (FIG. 6A) and efficacy (RTT phenotype score, FIG. 6B) for AAV9-RTT254 treated KO animals compared with vehicle-treated KO animals.

FIGS. 7A-F depict breakdown of the individual components of the RTT score, including graphic results of improved motor and breathing phenotypes in AAV9-RTT254 treated KO mice compared to controls, at two doses (1×10¹¹ vg and 3×10¹¹ vg).

FIG. 8 is a plasmid map depicting the elements of construct SEQ ID NO:14 (RTT252_CBE-ffluc1-hsaMECP2-3×binding-SV40 pA).

FIG. 9 is a plasmid map depicting the elements of construct SEQ ID NO:15 (RTT253_CBE-ffluc1-hsaMECP2-3×binding-WPRE3-SV40 pA).

FIG. 10 is a plasmid map depicting the elements of construct SEQ ID NO:16 (RTT254_CBM-ffluc1-hsaMECP2-3×binding-WPRE3-SV40 pA).

FIG. 11 is a plasmid map depicting the elements of construct SEQ ID NO:17 (RTT269_CBE-ffluc1-hsaMECP2-3×binding_mut3-WPRE3-SV40 pA).

FIG. 12 is a plasmid map depicting the elements of construct SEQ ID NO:18 (RTT270_CBE-ffluc1-hsaMECP2-3×binding_mut6-WPRE3-SV40 pA).

FIG. 13 is a plasmid map depicting the elements of construct SEQ ID NO:19 (RTT271_CBE-ran1g-hsaMECP2-3×binding-WPRE3-SV40 pA).

FIG. 14 is a plasmid map depicting the elements of construct SEQ ID NO:20 RTT272_CBE-ran2g-hsaMECP2-3×binding-WPRE3-SV40 pA).

FIG. 15 is a graph showing survival curves for NGN-401 treated Mecp2^{-/y} mice compared with vehicle-treated mice.

FIG. 16 is a graph showing weekly assessments of bodyweight following ICV delivery of vehicle or NGN-401 at P0-2. Animals were weighed weekly beginning at P28. Group size numbers are shown in the figure legend.

FIG. 17 is a graph showing weekly assessment of RTT phenotype score after ICV delivery of vehicle or NGN-401 at P0-2. Animals were scored weekly from 0 (normal) to 5 (most severe) in each parameter, beginning at P28 (age 4 weeks). Scores were combined to give an aggregate RTT phenotype score. Group size numbers are shown in the figure legend.

FIG. 18 is a survival curve for in-life safety for 26 weeks for WT mice treated with vehicle and Mecp2^{+/-} mice treated with NGN-401 or AAV9-RTT251 at either 1.0×10¹¹ vg/mouse or 3.0×10¹¹ vg/mouse.

FIG. 19 is a graph showing weekly assessment of bodyweight for NGN-401 treated Mecp2^{+/-} mice and vehicle treated WT and Mecp2^{+/-} mice.

FIG. 20 is a graph showing weekly assessment of MeCP2 overexpression toxicity of regulated NGN-401 or unregulated AAV9-RTT251 vectors at P1/2 in Mecp2^{+/-} female mice.

FIG. 21 is a graph showing vector biodistribution for NGN-401 treated Mecp2^{+/-} mice.

FIG. 22 is a graph showing vector biodistribution for AAV9-RTT251 treated Mecp2^{+/-} mice.

FIG. 23A-C are graphs of western blot protein expression data for NGN-401 treated Mecp2^{+/-} mice in cortex (FIG. 24A), cerebellum (FIG. 24B), and liver FIG. 24C).

FIG. 24A-B are graphs of western blot protein expression data for AAV9-RTT251 treated Mecp2^{+/-} mice in cortex (FIG. 25A) and liver FIG. 25C).

FIG. 25 shows in-life survival data following ICV administration of NGN-401 at P1/2 in Mecp2^{+/-} female mice at a dose of 7.4×10¹¹ vg/mouse.

FIG. 26 is a graph showing in-life bodyweight data following ICV administration of NGN-401 at P1/2 in Mecp2^{+/-} female mice at a dose of 7.4×10¹¹ vg/mouse. Mice were assessed weekly after P28 (age 4 weeks).

FIG. 27 is a graph showing MeCP2 overexpression toxicity score data following ICV administration of NGN-401 at P1/2 in Mecp2^{+/-} female mice at a dose of 7.4×10¹¹ vg/mouse. Mice were assessed weekly after P28 (age 4 weeks).

FIG. 28 is a graph showing in-life data RTT phenotype score data following ICV administration of NGN-401 at P1/2 in Mecp2^{+/-} female mice. Mice were assessed weekly after P28 (age 4 weeks).

FIG. 29 is a graph showing vector DNA biodistribution levels at 8-week timepoint in cortex, cerebellum, thoracic spinal cord, and liver after ICV delivery of NGN-401 at a dose of 7.4×10¹¹ vg/mouse. Results are presented as number of vector genome copies per diploid genome determined by a qPCR assay targeting the WPRE3 element of the NGN-401 vector and normalized per diploid genome using an assay targeting the mouse actin gene. Group size numbers are shown in the figure legend. vg=vector genomes.

FIG. 30 is a graph showing western blot quantification of MeCP2 protein levels at 8-week timepoint in cortex, cerebellum, and liver after ICV delivery of NGN-401 at a dose of 7.4×10¹¹ vg/mouse. Results are presented as the ratio of MeCP2 levels compared to levels in vehicle treated Mecp2^{+/-} mice. Group size numbers are in figure legend.

FIG. 31 is a graph showing data of transgene mRNA levels in NHPs treated with NGN-401 or AAV9-RTT251 at 5.0×10¹¹ or 1.5×10¹² vg/g brain weight, evaluated at Day 29/30 after dosing. mRNA levels in treated NHPs were determined via qRT-PCR using an assay targeting the WPRE3 element of the MECP2 transcript generated by the NGN-401 vector. Transgene mRNA levels are plotted for each animal relative to the average of the NGN-401 low dose group. n=3/group. vg=vector genomes.

### DETAILED DESCRIPTION

Rett syndrome (RTT) is a neurological disorder caused by mutations in the X-linked MECP2 gene. Mecp2-null mice recapitulate the cardinal features of the disorder and gene reactivation studies using conditional alleles lead to robust phenotypic correction. Whilst this makes RTT an attractive gene therapy target, MECP2 is a dosage sensitive gene with both animal studies and the human duplication disorder suggesting that MeCP2 levels need to be kept within a narrow range to achieve efficacy while avoiding overexpression related toxicity. These challenges are magnified by the biodistribution pattern of commonly used AAV vectors, which lead to hotspots of expression as well as excessive transgene expression in sensitive cell types.

To overcome these challenges, Applicants have developed optimized polynucleotide cassettes utilizing a single gene circuit in which transgene expression is regulated by a miRNA-based feedforward loop. This circuit provides a cell autonomous mechanism to prevent overexpression in strongly transduced cells whilst still allowing expression of therapeutic protein levels in more modestly transduced targets. Importantly, the miRNA sequence is not based on any existing mammalian miRNA thus preventing interference with endogenous miRNA-mRNA gene regulation in transduced cells. Optimized therapeutic polynucleotide MECP2 constructs and methods for treating a Rett syndrome and related disorders in a subject are provided. Coding sequences, including splice variants for human MECP2 can be found at the NCBI database as Gene ID 4204.

In certain embodiments, utilizing the wildtype MEPC2 gene in the therapeutic constructs described herein, exhibits improved protein expression, e.g., the protein encoded thereby is expressed at a more desirable or favorable level in a cell compared with the level of expression of the protein provided by various codon optimized MECP2 in an otherwise identical therapeutic polynucleotide cassette.

### Definitions

Unless otherwise defined, all technical and scientific terms used herein have the meaning commonly understood by one of ordinary skill in the art to which this invention belongs. The terminology used herein is for the purpose of describing particular embodiments only and is not intended to be limiting of the invention. The following terms have the meanings given:

AAV “rep” and “cap” genes refer to polynucleotide sequences encoding replication and encapsidation proteins of adeno-associated virus. AAV rep and cap are referred to herein as AAV “packaging genes.”

“AAV” is an abbreviation for adeno-associated virus and may be used to refer to the virus itself or modifications, derivatives, or pseudotypes thereof. The term covers all subtypes and both naturally occurring and recombinant forms, except where required otherwise. The abbreviation “rAAV” refers to recombinant adeno-associated virus. The term “AAV” includes AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV 8), AAV type 9 (AAV9), avian AAV, bovine AAV, canine AAV, equine AAV, primate AAV, non-primate AAV, and ovine AAV, and modifications, derivatives, or pseudotypes thereof. “Primate AAV” refers to AAV that infect primates, “non-primate AAV” refers to AAV that infect non-primate mammals, “bovine AAV” refers to AAV that infect bovine mammals, etc. In some embodiments, the AAV particle is AAV1, AAV2, rAAV3, AAV4, AAV5, AAV6, AAV7, AAV8, AAV9, AAV10, AAV11, AAV12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16. In some embodiments, the rAAV particle is a derivative, modification, or pseudotype of AAV1, AAV2, rAAV3, AAV4, AAV5, AAV6, AAV7, AAV8, AAV9, AAV 10, AAV11, AAV 12, AAV 13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV-PHP.B, AAV-PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

The various serotypes of AAV are attractive for several reasons, most prominently that AAV is believed to be non-pathogenic and that the wildtype virus can integrate its genome site-specifically into human chromosome 19 (Linden et al., 1996, Proc Natl Acad Sci USA 93:11288-11294). The insertion site of AAV into the human genome is called AAVS1. Site-specific integration, as opposed to random integration, is believed to likely result in a predictable long-term expression profile.

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC-002077 (AAV-1), AF063497 (AAV-1), NC-001401 (AAV-2), AF043303 (AAV-2), NC-001729 (AAV-3), NC-001829 (AAV-4), U89790 (AAV-4), NC-006152 (AAV-5), AF513851 (AAV-7), AF513852 (AAV-8), and NC-006261 (AAV-8); the disclosures of which are incorporated by reference herein. See also, e.g., Srivistava et al., 1983, J. Virology 45:555; Chiorini et al., 1998, J. Virology 71:6823; Chiorini et al., 1999, J. Virology 73: 1309; Bantel-Schaal et al., 1999, J. Virology 73:939; Xiao et al., 1999, J. Virology 73:3994; Muramatsu et al., 1996, Virology 221:208; Shade et al., 1986, J. Virol. 58:921; Gao et al., 2002, Proc. Nat. Acad. Sci. USA 99: 11854; Moris et al., 2004, Virology 33:375-383; international patent publications WO 00/28061, WO 99/61601, WO 98/11244; WO 2013/063379; WO 2014/194132; WO 2015/121501, and U.S. Pat. Nos. 6,156,303 and 7,906,111.

An “rAAV vector” as used herein refers to an AAV vector comprising a polynucleotide sequence not of AAV origin (i.e., a polynucleotide heterologous to AAV), typically a sequence of interest for the genetic transformation of a cell. In some embodiments, the heterologous polynucleotide may be flanked by at least one, and sometimes by two, AAV inverted terminal repeat sequences (ITRs). The term rAAV vector encompasses both rAAV vector particles and rAAV vector plasmids. A rAAV vector may either be single-stranded (ssAAV) or self-complementary (scAAV). An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as a “rAAV vector particle” or simply an “rAAV vector”. Thus, production of rAAV particle necessarily includes production of rAAV vector, as such a vector is contained within an rAAV particle.

“Vector,” means a recombinant plasmid or virus that comprises a polynucleotide to be delivered into a host cell, either in vitro or in vivo.

“Recombinant,” as used herein means that the vector, polynucleotide, polypeptide or cell is the product of various combinations of cloning, restriction or ligation steps (e.g. relating to a polynucleotide or polypeptide comprised therein), and/or other procedures that result in a construct that is distinct from a product found in nature. A recombinant virus or vector is a viral particle comprising a recombinant polynucleotide. The terms respectively include replicates of the original polynucleotide construct and progeny of the original virus construct.

“Recombinant viral vector” means a recombinant polynucleotide vector comprising one or more heterologous sequences (i.e., polynucleotide sequence not of viral origin).

“Recombinant”, as applied to an AAV particle means that the AAV particle is the product of one or more procedures that result in an AAV particle construct that is distinct from an AAV particle in nature.

“AAV Rep” means AAV replication proteins and analogs thereof.

“AAV Cap” means AAV capsid proteins, VP1, VP2 and VP3 and analogs thereof. In wild type AAV virus, three capsid genes vp1, vp2 and vp3 overlap each other. See, Grieger and Samulski, 2005, J. Virol. 79(15):9933-9944. A single P40 promoter allows all three capsid proteins to be expressed at a ratio of about 1:1:10, vp1, vp2, vp3, respectively, which complement with rAAV production. For the production of recombinant AAV vectors, desired ratio of VP1:VP2:VP3 is in the range of about 1:1:1 to about 1:1:100, preferably in the range of about 1:1:2 to about 1:1:50, more preferably in the range of about 1:1:5 to about 1:1:20. Although the desired ratio of VP1:VP2 is 1:1, the ratio range of VP1:VP2 could vary from 1:50 to 50:1.

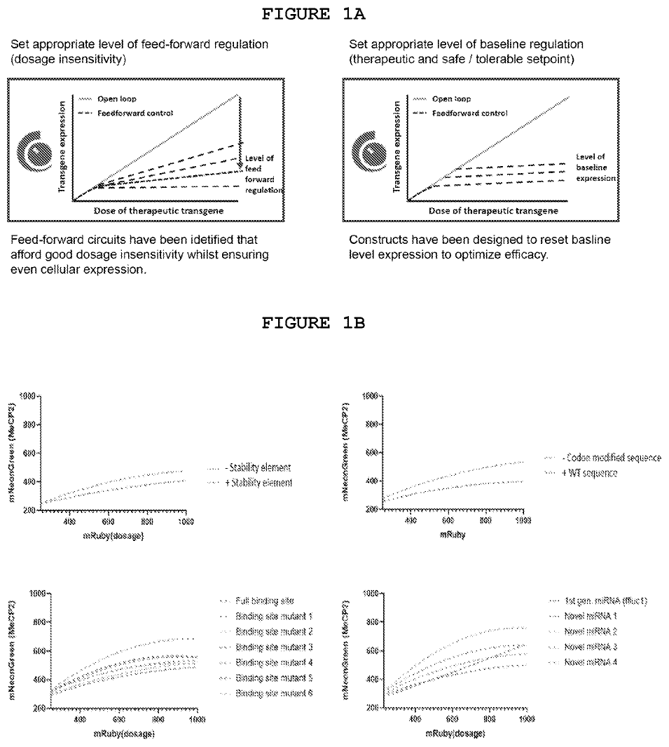

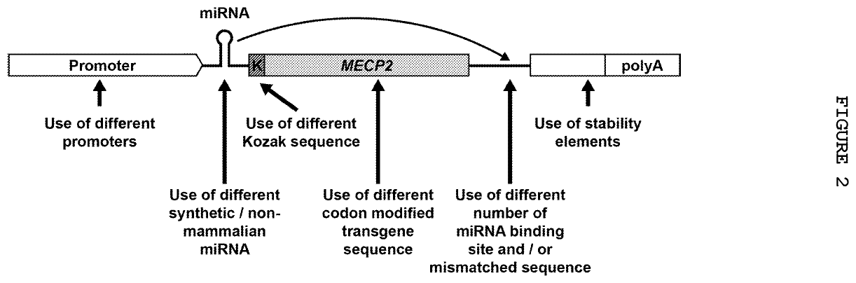

A comprehensive list and alignment of amino acid sequences of capsids of known AAV serotypes is provided by Marsic et al., 2014, Molecular Therapy 22(11):1900-1909, especially at supplementary FIG. 1.

For illustrative purposes only, wild type AAV2 comprises a small (20-25 nm) icosahedral virus capsid of AAV composed of three proteins (VP1, VP2, and VP3; a total of 60 capsid proteins compose the AAV capsid) with overlapping sequences. The proteins VP1 (735 aa; Genbank Accession No. AAC03780), VP2 (598 aa; Genbank Accession No. AAC03778) and VP3 (533 aa; Genbank Accession No. AAC03779) exist in a 1:1:10 ratio in the capsid. That is, for AAVs, VP1 is the full-length protein and VP2 and VP3 are progressively shorter versions of VP1, with increasing truncation of the N-terminus relative to VP1.

“AAV TR” means a palindromic terminal repeat sequence at or near the ends of the AAV genome, comprising mostly complementary, symmetrically arranged sequences, and includes analogs of native AAV TRs and analogs thereof. In the case of recombinant parvovirus vectors, the recombinant polynucleotide is flanked by at least one, preferably two, inverted terminal repeat sequences (ITRs).

“Cis-motifs” includes conserved sequences such as found at or close to the termini of the genomic sequence and recognized for initiation of replication; cryptic promoters or sequences at internal positions likely used for transcription initiation, splicing or termination.

“Therapeutically effective amount” means a minimal amount of active agent which is necessary to impart therapeutic benefit to a subject. For example, a “therapeutically effective amount” to a patient is such an amount which induces, ameliorates, stabilizes, slows down the progression or otherwise causes an improvement in the pathological symptoms, disease progression or physiological conditions associated with or resistance to succumbing to a disorder.

“Gene” means a polynucleotide containing at least one open reading frame that is capable of encoding a particular polypeptide or protein after being transcribed and translated.

“Coding sequence” means a sequence which encodes a particular protein” or “encoding nucleic acid”, denotes a nucleic acid sequence which is transcribed (in the case of DNA) and translated (in the case of mRNA) into a polypeptide in vitro or in vivo when placed under the control of (operably linked to) appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

“Chimeric” means, with respect to a viral capsid or particle, that the capsid or particle includes sequences from different parvoviruses, preferably different AAV serotypes, as described in Rabinowitz et al., U.S. Pat. No. 6,491,907, the disclosure of which is incorporated in its entirety herein by reference. See also Rabinowitz et al., 2004, J. Virol. 78(9):4421-4432. A particularly preferred chimeric viral capsid is the AAV2.5 capsid, which has the sequence of the AAV2 capsid with the following mutations: 263 Q to A; 265 insertion T; 705 N to A; 708 V to A; and 716 T to N. wherein the nucleotide sequence encoding such capsid is defined as SEQ ID NO: 15 as described in WO 2006/066066. Other preferred chimeric AAVs include, but are not limited to, AAV2i8 described in WO 2010/093784, AAV2G9 and AAV8G9 described in WO 2014/144229, and AAV9.45 (Pulicherla et al., 2011, Molecular Therapy 19(6):1070-1078).

“Flanked,” with respect to a sequence that is flanked by other elements, indicates the presence of one or more the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence (e.g., a transgene) that is “flanked” by two other elements (e.g., TRs), indicates that one element is located 5′ to the sequence and the other is located 3′ to the sequence; however, there may be intervening sequences there between.

“Polynucleotide” means a sequence of nucleotides connected by phosphodiester linkages. Polynucleotides are presented herein in the direction from the 5′ to the 3′ direction. A polynucleotide of the present invention can be a deoxyribonucleic acid (DNA) molecule or ribonucleic acid (RNA) molecule. Where a polynucleotide is a DNA molecule, that molecule can be a gene or a cDNA molecule. Nucleotide bases are indicated herein by a single letter code: adenine (A), guanine (G), thymine (T), cytosine (C), inosine (I) and uracil (U). A polynucleotide of the present invention can be prepared using standard techniques well known to one of skill in the art.

“Transduction” of a cell by a virus means that there is transfer of a nucleic acid from the virus particle to the cell.

“Codon optimized MECP2” means a modified nucleic acid encoding the MECP2 gene with at least one modification compared with a wild-type nucleic acid encoding MECP2 (SEQ ID NO: 7), wherein the modification includes, but is not limited to, decreased GC content or an MECP2 gene with a reduced CpG content. Human MECP2 can be found at the NCBI database as Gene ID 4204 (considered wildtype).

“Transfection” of a cell means that genetic material is introduced into a cell for the purpose of genetically modifying the cell. Transfection can be accomplished by a variety of means known in the art, such as calcium phosphate, polyethyleneimine, electroporation, and the like.

“Polypeptide” encompasses both peptides and proteins, unless indicated otherwise.

“Gene transfer” or “gene delivery” refers to methods or systems for reliably inserting foreign nucleic acid, e.g. DNA or RNA into host cells. Such methods can result in transient expression of non-integrated transferred DNA, extrachromosomal replication and expression of transferred replicons (e.g. episomes), or integration of transferred genetic material into the genomic DNA of host cells.

“Transgene” is used to mean any heterologous nucleotide sequence incorporated in a vector, including a viral vector, for delivery to and including expression in a target cell (also referred to herein as a “host cell”), and associated expression control sequences, such as promoters. It is appreciated by those of skill in the art that expression control sequences will be selected based on ability to promote expression of the transgene in the target cell. An example of a transgene is a nucleic acid encoding a therapeutic polypeptide.

The term “cell culture,” refers to cells grown adherent or in suspension, bioreactors, roller bottles, hyperstacks, microspheres, macrospheres, flasks and the like, as well as the components of the supernatant or suspension itself, including but not limited to rAAV particles, cells, cell debris, cellular contaminants, colloidal particles, biomolecules, host cell proteins, nucleic acids, and lipids, and flocculants. Large scale approaches, such as bioreactors, including suspension cultures and adherent cells growing attached to microcarriers or macrocarriers in stirred bioreactors, are also encompassed by the term “cell culture.” Cell culture procedures for both large and small-scale production of proteins are encompassed by the present disclosure.

The terms “purifying”, “purification”, “separate”, “separating”, “separation”, “isolate”, “isolating”, or “isolation”, as used herein, refer to increasing the degree of purity of rAAV particles from a sample comprising the target product and one or more impurities. Typically, the degree of purity of the target product is increased by removing (completely or partially) at least one impurity from the sample. In some embodiments, the degree of purity of the rAAV in a sample is increased by removing (completely or partially) one or more impurities from the sample by using a method described herein.

“Homologous” used in reference to peptides, refers to amino acid sequence similarity between two peptides. When an amino acid position in both of the peptides is occupied by identical amino acids, they are homologous at that position. Thus by “substantially homologous” means an amino acid sequence that is largely, but not entirely, homologous, and which retains most or all of the activity as the sequence to which it is homologous.

As used herein, “substantially homologous” as used herein means that a sequence is at least 50% identical, and preferably at least 75% and more preferably 95% homology to the reference peptide. Additional peptide sequence modification are included, such as minor variations, deletions, substitutions or derivatizations of the amino acid sequence of the sequences disclosed herein, so long as the peptide has substantially the same activity or function as the unmodified peptides. Derivatives of an amino acid may include but not limited to trifluoroleucine, hexafluoroleucine, 5,5,5-trifluoroisoleucine, 4,4,4-trifluorovaline, p-fluorophenylaline, o-fluorotyrosine, m-fluorotyrosine, 2,3-difluorotyrosine, 4-fluorohistidine, 2-fluorohistidine, 2,4-difluorohistidine, fluoroproline, difluoroproline, 4-hydroxyproline, selenomethionine, telluromethionine, selenocysteine, selenatryptophans, 4-aminotryptophan, 5-aminotryptophan, 5-hydroxytryptophan, 7-azatryptophan, 4-fluorotryptophan, 5-fluorotryptophan, 6-fluorotryptophan, homoallylglycine, homopropargylglycine, 2-butynylglycine, cis-crotylglycine, allylglycine, dehydroleucine, dehydroproline, 2-amino-3-methyl-4-pentenoic acid, azidohomoalanine, asidoalanine, azidonorleucine, p-ethynylphenylalanine, p-azidophenylalanine, p-bromophenylalanine, p-acetylphenylalanine and benzofuranylalanine. Notably, a modified peptide will retain activity or function associated with the unmodified peptide, the modified peptide will generally have an amino acid sequence “substantially homologous” with the amino acid sequence of the unmodified sequence.

In certain embodiments, the therapeutic polynucleotide construct contains a wild-type MECP2 gene. In alternative embodiments, provided herein are modified MECP2 genes. Additional embodiments provided herein include nucleic acid constructs, such as vectors, which include as part of their sequence a modified MECP2 gene, e.g., GC content optimized MECP2 gene sequence comprising a greater or lesser amount of GC nucleotides compared with the wild type MECP2 gene sequence and/or a MECP2 gene sequence having reduced, or increased, levels of CpG dinucleotides compared with the level of CpG dinucleotides present in the wild type MECP2 gene. For example, embodiments include plasmids and/or other vectors that include either the wildtype or the modified MECP2 sequence along with other elements, such as regulatory elements. Further embodiments provide packaged gene delivery vehicle, such as a viral capsid, including either the wildtype or the modified MECP2 sequence. Provided herein are also methods of delivery and, preferably, expressing the wildtype or modified MECP2 gene by delivering the modified sequence into a cell along with elements required to promote expression in the cell. The invention also provides gene therapy methods in which the wildtype or modified MECP2 gene sequence is administered to a subject, e.g., as a component of a vector and/or packaged as a component of a viral gene delivery vehicle. Particular embodiments include those wherein the modified nucleic acid sequence has an identity of 90% to SEQ ID NO: 7 (wildtype human MECP2). In certain embodiments the MECP2 construct exhibits greater than 90, 91, 92, 93, 94, 95, 96, 97, 98, or 99% identity with SEQ ID NO: 7 (wildtype human MECP2).

Treatment may, for example, be affected to increase levels of MeCP2 in the subject in an amount which provides a therapeutic level of MeCP2 without undesired toxicity effects.

### Modified Nucleic Acid for Expression of MECP2

“Optimized” or “codon-optimized” as referred to interchangeably herein, refer to a coding sequence that has been optimized relative to a wild type coding sequence (e.g., a coding sequence for MECP2) to increase expression of the coding sequence, e.g., by minimizing usage of rare codons, decreasing the number of CpG dinucleotides, removing cryptic splice donor or acceptor sites, removing Kozak sequences, removing ribosomal entry sites, and the like.

“Percentage identity” as used herein, refers to the numerical score of two given polynucleotides and/or polypeptides that have identical nucleic and/or amino acids within the same position as given by a typical sequence alignment program (i.e. BLAST methods). Suitably “percentage identity” is determine over a complete length of a polynucleotide and/polypeptide or over the length of a functional fragment of a polynucleotide and/or polypeptide. A functional fragment may be provided as a shorter part of the polynucleotide and/or polypeptide which provides a same desired function as a complete length of polynucleotide and/or polypeptide.

### Codon Optimization

There are sixty-four different codons. Sixty-one of them encode the twenty standard amino acids, while another three function as stop codons. The greater number of codons relative to the number of amino acids they code for, means that a single amino acid can be encoded by more than one codon. Indeed, some common amino acids, such as arginine and leucine, are encoded by as many as 6 codons.

Different organisms exhibit bias towards use of certain codons over others for the same amino acid. Some species are known to avoid certain codons almost entirely. Such biases may affect protein expression. Therefore, in certain embodiments it can be useful to consider codon optimization when design gene therapy constructs.

While numerous factors contribute to the success of protein expression, codon optimization plays a critical role, particularly when proteins are expressed in a heterologous system. As an example, if a human gene is to be expressed in E. coli, choosing codons preferentially used by the bacterium can increase the success of protein expression. This is particularly true when rare codons are eliminated.

In certain embodiments as described herein below, it has been determined that the wildtype MECP2 coding sequence provides optimal moderate expression in certain of the therapeutic cassettes, as shown and described herein.

### Sequence Modifications and Polynucleotide Cassette Elements

Examples of modifications include elimination of one or more cis-acting motifs and introduction of one or more Kozak sequences. In one embodiment, one or more cis-acting motifs are eliminated and one Kozak sequence is introduced.

Examples of cis acting motifs that may be eliminated include internal TATA-boxes; chi-sites; ribosomal entry sites; ARE, INS, and/or CRS sequence elements; repeat sequences and/or RNA secondary structures; (cryptic) splice donor and/or acceptor sites, branch points; and restriction sites, (e.g., Sall).

In certain embodiments, the MeCP2 gene sequence may also include flanking restriction sites to facilitate subcloning into expression vector. Many such restriction sites are well known in the art.

The disclosure includes a nucleic acid vector including the MECP2 gene sequence and various regulatory or control elements. The precise nature of regulatory elements useful for gene expression will vary from organism to organism and from cell type to cell type. In general, they include a promoter which directs the initiation of RNA transcription in the cell of interest. The promoter may be constitutive or regulated. Constitutive promoters are those which cause an operably linked gene to be expressed essentially at all times. Regulated promoters are those which can be activated or deactivated. Regulated promoters include inducible promoters, which are usually “off” but which may be induced to turn “on,” and “repressible” promoters, which are usually “on” but may be turned “off” Many different regulators are known, including temperature, hormones, cytokines, heavy metals and regulatory proteins. The distinctions are not absolute; a constitutive promoter may often be regulated to some degree. In some cases, an endogenous pathway may be utilized to provide regulation of the transgene expression, e.g., using a promoter that is naturally downregulated when the pathological condition improves.

Examples of suitable promoters include adenoviral promoters, such as the adenoviral major late promoter; heterologous promoters, such as the cytomegalovirus (CMV) promoter; the respiratory syncytial virus promoter; the Rous Sarcoma Virus (RSV) promoter; the albumin promoter; inducible promoters, such as the Mouse Mammary Tumor Virus (MMTV) promoter; the metallothionein promoter; heat shock promoters; the α-1-antitrypsin promoter; the hepatitis B surface antigen promoter; the transferrin promoter; the apolipoprotein A-1 promoter; chicken beta-actin (CBA) promoter, the CBh promoter, and the CAG promoter (cytomegalovirus early enhancer element and the promoter, the first exon, and the first intron of chicken beta-actin gene and the splice acceptor of the rabbit beta-globin gene) (Alexopoulou et al., 2008, BioMed. Central Cell Biol. 9:2), the CBE promoter (cytomegalovirus early enhancer element and the promoter of chicken beta-actin, and the first exon, first intron, and second exon of human elongation factor 1 alpha), the CBM promoter (cytomegalovirus early enhancer element and the promoter of chicken beta-actin, and the first exon, first intron, and second exon of MINIX) and human MECP2 promoters. The promoter may be a tissue-specific promoter, such as the mouse albumin promoter, which is active in liver cells as well as the transthyretin promoter (TTR). In certain embodiments, liver detargeted promoters can be used. It will be clear to one skilled in the art how to utilize and adapt any of these features as described herein.

In another aspect, the modified nucleic acid encoding MECP2 further comprises an enhancer to increase expression of the protein. Many enhancers are known in the art, including, but not limited to, the cytomegalovirus major immediate-early enhancer. More specifically, the CMV MIE promoter comprises three regions: the modulator, the unique region and the enhancer (Isomura and Stinski, 2003, J. Virol. 77(6):3602-3614). The CMV enhancer region can be combined with other promoters, or a portion thereof, to form hybrid promoters to further increase expression of a nucleic acid operably linked thereto. For example, a chicken beta-actin (CBA) promoter, or a portion thereof, can be combined with the CMV promoter/enhancer, or a portion thereof, and a hybrid intron of chicken beta-actin (CBA) and minute virus of mice (MMV) introns to make a version of CBA termed the “CBh” promoter, which stands for chicken beta-actin hybrid promoter, as described in Gray et al. (2011, Human Gene Therapy 22:1143-1153).

In some embodiments, a synthetic RNA circuit may be used to regulate expression of the transgene. The circuit includes a single-gene microRNA (miRNA)-based feed-forward loop. It provides a non-mammalian, or synthetic (not naturally occurring) miRNA expressed within an intron that targets its own transcript, wherein the miRNAs are not expected to target human mRNAs. The miRNA is non-mammalian or synthetic. Expressing the miRNA from within different introns (hEF1a vs MINIX) may be used to fine-tune the circuit. Expressing different miRNAs (EXACT1 vs EXACT2 vs EXACT3) may be used to fine-tune the circuit. Binding sites in the 3′UTR of the construct mRNA are specific to the non-mammalian or synthetic miRNA being expressed from within the intron of the circuit and provide control of the expression of the transgene. The non-mammalian or synthetic miRNA binding sites are not expected to allow binding of any human endogenous miRNAs. Providing different numbers of miRNA binding sites (one or more) may be used to fine-tune the circuit.

Introns can also be used to increase efficiency in mammalian expression vectors. Examples of introns are murine cytomegalovirus (MCMV) immediate early (IE) promoter, human cytomegalovirus (HCMV) immediate early (IE) promoter, and human elongation factor one alpha (EF-1 alpha) promoter. The intron can be varied depending on the gene of interest.

Further, the control elements can include a collagen stabilization sequence (CSS), a stop codon, a termination sequence, and a poly-adenylation signal sequence, such as, but not limited to a bovine growth hormone poly A signal sequence (bGHpA), to drive efficient addition of a poly-adenosine “tail” at the 3′ end of a eukaryotic mRNA (see, e.g., Goodwin and Rottman, 1992, J. Biol. Chem. 267(23):16330-16334).

The poly-A tail is a long chain of adenine nucleotides that is added to a messenger RNA (mRNA) molecule during RNA processing to increase the stability of the molecule. Similar to what happens in vivo. The poly-A tail makes the RNA molecule more stable and prevents its degradation. Additionally, the poly-A tail allows the mature messenger RNA molecule to be exported from the nucleus and translated into a protein by ribosomes in the cytoplasm.

The woodchuck hepatitis virus post-transcriptional regulatory element (WPRE) increases transgene expression from a variety of viral vectors. WPRE is most effective when placed downstream of the transgene, proximal to the polyadenylation signal. It is possible that WPRE reduces viral mRNA readthrough transcription by improving transcript termination, which in turn would increase viral titers and expression. (Gene Therapy volume 14, pages 1298-1304 (2007)).

### Non-Viral Vectors

In a particular embodiment, the vector used according to the invention is a non-viral vector. Typically, the non-viral vector may be a plasmid which includes nucleic acid sequences encoding the MECP2, or variants thereof.

### Packaged MECP2 Sequence

The MECP2 gene sequence may also be provided as a component of a packaged viral vector. In general, packaged viral vectors include a viral vector packaged in a capsid. Viral vectors and viral capsids are discussed in the ensuing sections. The nucleic acid packaged in the rAAV vector can be single-stranded (ss), self-complementary (sc), or double-stranded (ds). It is expected that the construct comprising any of the polynucleotide constructs described herein is capable of desired packaging and expression. Additionally, the single stranded vector exhibits similarly desirable packing capabilities.

### Viral Vector

Typically, viral vectors carrying transgenes are assembled from polynucleotides encoding the transgene, suitable regulatory elements and elements necessary for production of viral proteins which mediate cell transduction. Examples of a viral vector include but are not limited to adenoviral, retroviral, lentiviral, herpesvirus and adeno-associated virus (AAV) vectors.

The viral vector component of the packaged viral vectors produced according to the methods of the invention includes at least one transgene, e.g., a MECP2 gene sequence and associated expression control sequences for controlling expression of the modified MECP2 therapeutic cassette.

In a preferred embodiment, the viral vector includes a portion of a parvovirus genome, such as an AAV genome with rep and cap deleted and/or replaced by the MECP2 gene sequence and its associated expression control sequences. The MECP2 gene sequence is typically inserted adjacent to one or two (i.e., is flanked by) AAV TRs or TR elements adequate for viral replication (Xiao et al., 1997, J. Virol. 71(2): 941-948), in place of the nucleic acid encoding viral rep and cap proteins. Other regulatory sequences suitable for use in facilitating tissue-specific expression of the MECP2 cassette in the target cell may also be included.

One skilled in the art would appreciate that an AAV vector comprising a transgene and lacking virus proteins needed for viral replication (e.g., cap and rep), cannot replicate since such proteins are necessary for virus replication and packaging. Further, AAV is a Dependovirus in that it cannot replicate in a cell without co-infection of the cell by a helper virus. Helper viruses include, typically, adenovirus or herpes simplex virus. Alternatively, as discussed below, the helper functions (E1a, E1b, E2a, E4, and VA RNA) can be provided to a packaging cell including by transfecting the cell with one or more nucleic acids encoding the various helper elements and/or the cell can comprise the nucleic acid encoding the helper protein. For instance, HEK 293 were generated by transforming human cells with adenovirus 5 DNA and now express a number of adenoviral genes, including, but not limited to E1 and E3 (see, e.g., Graham et al., 1977, J. Gen. Virol. 36:59-72). Thus, those helper functions can be provided by the HEK 293 packaging cell without the need of supplying them to the cell by, e.g., a plasmid encoding them.

The viral vector may be any suitable nucleic acid construct, such as a DNA or RNA construct and may be single stranded, double stranded, or duplexed (i.e., self-complementary as described in WO 2001/92551).

One skilled in the art would appreciate that an rAAV vector can further include a “stuffer” or “filler” sequence (filler/stuffer) where the nucleic acid comprising the transgene is less than the approximately 4.1 to 4.9 kb size for optimal packaging of the nucleic acid into the AAV capsid. See, Grieger and Samulski, 2005, J. Virol. 79(15):9933-9944. That is, AAV vectors typically accept inserts of DNA having a defined size range which is generally about 4 kb to about 5.2 kb, or slightly more. Thus, for shorter sequences, inclusion of a filler/stuffer in the insert fragment in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. In particular embodiments of an rAAV vector, a heterologous polynucleotide sequence has a length less than 4.7 Kb and the filler/stuffer polynucleotide sequence has a length that when combined (e.g., inserted into a vector) with the heterologous polynucleotide sequence has a total length between about 3.0-5.5 Kb, or between about 4.0-5.0 Kb, or between about 4.3-4.8 Kb.

An intron can also function as a filler/stuffer polynucleotide sequence in order to achieve a length for AAV vector packaging into a virus particle. Introns and intron fragments that function as a filler/stuffer polynucleotide sequence also can enhance expression. For example, inclusion of an intron element may enhance expression compared with expression in the absence of the intron element (Kurachi et al., 1995, J. Biol. Chem. 270(10):5276-5281). Furthermore, filler/stuffer polynucleotide sequences are well known in the art and include, but are not limited to, those described in WO 2014/144486.

### Viral Capsid

The viral capsid component of the packaged viral vectors may be a parvovirus capsid. AAV Cap and chimeric capsids are preferred. Examples of suitable parvovirus viral capsid components are capsid components from the family Parvoviridae, such as an autonomous parvovirus or a Dependovirus. For example, the viral capsid may be an AAV capsid (e.g., AAV1, AAV2, AAV3, AAV4, AAV5, AAV6, AAV7 AAV8, AAV9, AAV10, AAV11, AAV12, AAV1.1, AAV2.5, AAV6.1, AAV6.3.1, AAV9.45, AAVrh10, AAVrh74, RHM4-1 (SEQ ID NO:5 of WO 2015/013313), AAV2-TT, AAV2-TT-S312N, AAV3B-S312N, AAV-LK03, AAVrh10, AAV-PHP.B, AAV-PHP.eB, AAV-PHP.S, AAV2.GL, AAV2.NN, snake AAV, avian AAV, bovine AAV, canine AAV, equine AAV, ovine AAV, goat AAV, shrimp AAV, and any other AAV now known or later discovered. see, e.g., Fields et al., VIROLOGY, volume 2, chapter 69 (4^{th} ed., Lippincott-Raven Publishers). Capsids may be derived from a number of AAV serotypes disclosed in U.S. Pat. No. 7,906,111; Gao et al., 2004, J. Virol. 78:6381; Moris et al., 2004, Virol. 33:375; WO 2013/063379; WO 2014/194132; and include true type AAV (AAV-TT) variants disclosed in WO 2015/121501, and RHM4-1, RHM15-1 through RHM15-6, and variants thereof, disclosed in WO 2015/013313, and one skilled in the art would know there are likely other variants not yet identified that perform the same or similar function, or may include components from two or more AAV capsids. A full complement of AAV Cap proteins includes VP1, VP2, and VP3. The ORF comprising nucleotide sequences encoding AAV VP capsid proteins may comprise less than a full complement AAV Cap protein or the full complement of AAV Cap proteins may be provided.

One or more of the AAV Cap proteins may be a chimeric protein, including amino acid sequences of AAV Caps from two or more viruses, preferably two or more AAVs, as described in Rabinowitz et al., U.S. Pat. No. 6,491,907, the entire disclosure of which is incorporated herein by reference. For example, the chimeric virus capsid can include an AAV1 Cap protein or subunit and at least one AAV2 Cap or subunit. The chimeric capsid can, for example, include an AAV capsid with one or more B19 Cap subunits, e.g., an AAV Cap protein or subunit can be replaced by a B19 Cap protein or subunit. For example, in a preferred embodiment, the Vp3 subunit of the AAV capsid can be replaced by the Vp2 subunit of B19.

Another embodiment includes chimeric viral strains synthesized include the combination of AAV backbones from AAV2, AAV3, AAV6, AAV8, etc., with a galactose (Gal) binding footprint from AAV9. Adeno-associated viruses (AAVs) are helper-dependent parvoviruses that exploit heparan sulfate (HS), galactose (Gal), or sialic acids (Sia) as primary receptors for cell surface binding. For instance, AAV serotypes 2 and 3b utilize HS. AAV1, 4, and 5 bind Sia with different linkage specificities, AAV serotype 6, which recognizes both Sia and HS, whereas AAV9 exploits Gal for host cell attachment. Specifically, the galactose (Gal) binding footprint from AAV9 was grafted onto the heparin sulfate-binding AAV serotype 2 and just grafting of orthogonal glycan binding footprints improves transduction efficiency. A new dual glycan-binding strain (AAV2G9) and a chimeric, muscle-tropic strain (AAV2i8G9) were generated by incorporating the Gal binding footprint from AAV9 into the AAV2 VP3 backbone or the chimeric AAV2i8 capsid template using structural alignment and site-directed mutagenesis. In vitro binding and transduction assays confirmed the exploitation of both HS and Gal receptors by AAV2G9 for cell entry. Subsequent in vivo characterization of the kinetics of transgene expression and vector genome biodistribution profiles indicate fast, sustained, and enhanced transgene expression by this rationally engineered chimeric AAV strain. A similar, improved transduction profile was observed with the liver-detargeted, muscle-specific AAV2i8G9 chimera (Shen, et al., 2013, J. Biol. Chem. 288(4):28814-28823). Such new grafting combination is fully described in WO2014/144229 the contents of which are incorporated by reference herein. Additional liver de-targeted AAVs, such as AAV9.45, are described in Pulicherla et al., 2011, Molecular Therapy 19(6):1070-1078, the contents of which are incorporated by reference as if set forth in their entirety herein.

In yet another embodiment the present invention provides for the use of ancestral AAV vectors for use in therapeutic in vivo gene therapy. Specifically, in silico-derived sequences were synthesized de novo and characterized for biological activities. This effort led to the generation of nine functional putative ancestral AAVs and the identification of Anc80, the predicted ancestor of AAV serotypes 1, 2, 8 and 9 (Zinn et al., 2015, Cell Reports 12:1056-1068). Predicting and synthesis of such ancestral sequences in addition to assembling into a virus particle may be accomplished by using the methods described in WO 2015/054653, the contents of which are incorporated by reference herein. Notably, the use of the virus particles assembled from ancestral viral sequences exhibit reduced susceptibility to pre-existing immunity in current day human population than do contemporary viruses or portions thereof.

### Production of Packaged Viral Vector

The invention includes packaging cells, which are encompassed by “host cells,” which may be cultured to produce packaged viral vectors of the invention. The packaging cells of the invention generally include cells with heterologous (1) viral vector function(s), (2) packaging function(s), and (3) helper function(s). Each of these component functions is discussed in the ensuing sections.

Initially, the vectors can be made by several methods known to skilled artisans (see, e.g., WO 2013/063379). A preferred method is described in Grieger, et al. 2015, Molecular Therapy 24(2):287-297, the contents of which are incorporated by reference herein for all purposes. Briefly, efficient transfection of HEK293 cells is used as a starting point, wherein an adherent HEK293 cell line from a qualified clinical master cell bank is used to grow in animal component-free suspension conditions in shaker flasks and WAVE bioreactors that allow for rapid and scalable rAAV production. Using the triple transfection method (e.g., WO 96/40240), the suspension HEK293 cell line generates greater than 1×10⁵ vector genome containing particles (vg)/cell or greater than X 10¹⁴ vg/L of cell culture when harvested 48 hours post-transfection. More specifically, triple transfection refers to the fact that the packaging cell is transfected with three plasmids: one plasmid encodes the AAV rep and cap genes, another plasmid encodes various helper functions (e.g., adenovirus or HSV proteins such as E1a, E1b, E2a, E4, and VA RNA, and another plasmid encodes the transgene and its various control elements (e.g., MECP2 gene and CBM or CBE promoter).

To achieve the desired yields, a number of variables are optimized such as selection of a compatible serum-free suspension media that supports both growth and transfection, selection of a transfection reagent, transfection conditions and cell density. A universal purification strategy, based on ion exchange chromatography methods, was also developed that resulted in high purity vector preps of AAV serotypes 1-6, 8, 9 and various chimeric capsids. This user-friendly process can be completed within one week, results in high full to empty particle ratios (>90% full particles), provides post-purification yields (>1×10¹³ vg/L) and purity suitable for clinical applications and is universal with respect to all serotypes and chimeric particles. This scalable manufacturing technology has been utilized to manufacture GMP Phase I clinical AAV vectors for retinal neovascularization (AAV2), Hemophilia B (scAAV8), Giant Axonal Neuropathy (scAAV9) and Retinitis Pigmentosa (AAV2), which have been administered into patients. In addition, a minimum of a 5-fold increase in overall vector production by implementing a perfusion method that entails harvesting rAAV from the culture media at numerous time-points post-transfection.

### Viral Vector Functions

The packaging cells of the invention include viral vector functions, along with packaging and vector functions. The viral vector functions typically include a portion of a parvovirus genome, such as an AAV genome, with rep and cap deleted and replaced by the wildtype or optimized MECP2 sequence and its associated expression control sequences. The viral vector functions include sufficient expression control sequences to result in replication of the viral vector for packaging. Typically, the viral vector includes a portion of a parvovirus genome, such as an AAV genome with rep and cap deleted and replaced by the transgene and its associated expression control sequences. The transgene is typically flanked by two AAV TRs, in place of the deleted viral rep and cap ORFs. Appropriate expression control sequences are included, such as a tissue-specific promoter and other regulatory sequences suitable for use in facilitating tissue-specific expression of the transgene in the target cell. The transgene is typically a nucleic acid sequence that can be expressed to produce a therapeutic polypeptide or a marker polypeptide.

“Duplexed vectors” may interchangeably be referred to herein as “dimeric” or “self-complementary” vectors. The duplexed parvovirus particles may, for example, comprise a parvovirus capsid containing a virion DNA (vDNA). The vDNA is self-complementary so that it may form a hairpin structure upon release from the viral capsid. The duplexed vDNA appears to provide to the host cell a double-stranded DNA that may be expressed (i.e., transcribed and, optionally, translated) by the host cell without the need for second-strand synthesis, as required with conventional parvovirus vectors. Duplexed/self-complementary rAAV vectors are well-known in the art and described, e.g., in WO 2001/92551, WO 2015/006743, and many others.

The viral vector functions may suitably be provided as duplexed vector templates, as described in U.S. Pat. No. 7,465,583 to Samulski et al. (the entire disclosure of which is incorporated herein by reference for its teaching regarding duplexed vectors). Duplexed vectors are dimeric self-complementary (sc) polynucleotides (typically, DNA). The duplexed vector genome preferably contains sufficient packaging sequences for encapsidation within the selected parvovirus capsid (e.g., AAV capsid). Those skilled in the art will appreciate that the duplexed vDNA may not exist in a double-stranded form under all conditions but has the ability to do so under conditions that favor annealing of complementary nucleotide bases. “Duplexed parvovirus particle” encompasses hybrid, chimeric and targeted virus particles. Preferably, the duplexed parvovirus particle has an AAV capsid, which may further be a chimeric or targeted capsid, as described above.

The viral vector functions may suitably be provided as duplexed vector templates, as described in U.S. Pat. No. 7,465,583 to Samulski et al. (the entire disclosure of which is incorporated herein by reference for its teaching regarding duplexed vectors). Duplexed vectors are dimeric self-complementary (sc) polynucleotides (typically, DNA). For example, the DNA of the duplexed vectors can be selected so as to form a double-stranded hairpin structure due to intrastrand base pairing. Both strands of the duplexed DNA vectors may be packaged within a viral capsid. The duplexed vector provides a function comparable to double-stranded DNA virus vectors and can alleviate the need of the target cell to synthesize complementary DNA to the single-stranded genome normally encapsulated by the virus.

The TR(s) (resolvable and non-resolvable) selected for use in the viral vectors are preferably AAV sequences, with serotypes 1, 2, 3, 4, 5 and 6 being preferred. Resolvable AAV TRs need not have a wild-type TR sequence (e.g., a wild-type sequence may be altered by insertion, deletion, truncation or missense mutations), as long as the TR mediates the desired functions, e.g., virus packaging, integration, and/or provirus rescue, and the like. The TRs may be synthetic sequences that function as AAV inverted terminal repeats, such as the “double-D sequence” as described in U.S. Pat. No. 5,478,745 to Samulski et al., the entire disclosure of which is incorporated in its entirety herein by reference. Typically, but not necessarily, the TRs are from the same parvovirus, e.g., both TR sequences are from AAV2

The packaging functions include capsid components. The capsid components are preferably from a parvoviral capsid, such as an AAV capsid or a chimeric AAV capsid function. Examples of suitable parvovirus viral capsid components are capsid components from the family Parvoviridae, such as an autonomous parvovirus or a Dependovirus. For example, the capsid components may be selected from AAV capsids, e.g., AAV1, AAV2, AAV3, AAV4, AAV5, AAV6, AAV7, AAV8, AAV9, AAV10, AAV11, AAV12, AAVrh10, AAVrh74, RHM4-1, RHM15-1, RHM15-2, RHM15-3/RHM15-5, RHM15-4, RHM15-6, AAV Hu.26, AAV1.1, AAV2.5, AAV6.1, AAV6.3.1, AAV9.45, AAV2i8, AAV2G9, AAV2i8G9, AAV2-TT AAV2-TT-S312N), AAV3B-S312N, and AAV-LK03 (See, U.S. Pat. No. 10,548,947, and other novel capsids as yet unidentified or from non-human primate sources. Capsid components may include components from two or more AAV capsids.

In another embodiment, one or more of the VP capsid proteins is a chimeric protein, comprising amino acid sequences from two or more viruses, preferably two or more AAVs, as described in Rabinowitz et al., U.S. Pat. No. 6,491,907. A chimeric capsid is described herein as having at least one amino acid residue from one serotype combined with another serotype that is sufficient to modify a) viral yield, b) immune response, c) targeting, d) de-targeting, etc.

Further chimeric proteins can be made by instruction set forth in Li, et al., 2008, Mol. Ther. 16(7):1252-1260, the contents of which are incorporated by reference herein. Specifically, a DNA shuffling-based approach was used for developing cell type-specific vectors through directed evolution. Capsid genomes of adeno-associated virus (AAV) serotypes 1-9 were randomly fragmented and reassembled using PCR to generate a chimeric capsid library. A single infectious clone (chimeric-1829) containing genome fragments from AAV1, 2, 8, and 9 was isolated from an integrin minus hamster melanoma cell line previously shown to have low permissiveness to AAV. Molecular modeling studies suggest that AAV2 contributes to surface loops at the icosahedral threefold axis of symmetry, while AAV1 and 9 contribute to two- and five-fold symmetry interactions, respectively. The C-terminal domain (AAV9) was identified as a critical structural determinant of melanoma tropism through rational mutagenesis. Chimeric-1829 utilizes heparan sulfate as a primary receptor and transduces melanoma cells more efficiently than all serotypes. Application of this technology to alternative cell/tissue types using AAV or other viral capsid sequences is likely to yield a new class of biological nanoparticles as vectors for human gene transfer.

The packaged viral vector generally includes the wild type or modified MECP2 sequence and expression control sequences flanked by TR elements, referred to herein as the “transgene” or “transgene expression cassette,” sufficient to result in packaging of the vector DNA and subsequent expression of the wildtype or modified MECP2 sequence in the transduced cell. The viral vector functions may, for example, be supplied to the cell as a component of a plasmid or an amplicon.

The viral vector functions may exist extra chromosomally within the cell line and/or may be integrated into the cell's chromosomal DNA.

Any method of introducing the nucleotide sequence carrying the viral vector functions into a cellular host for replication and packaging may be employed, including but not limited to, electroporation, calcium phosphate precipitation, microinjection, cationic or anionic liposomes, and liposomes in combination with a nuclear localization signal. In embodiments wherein the viral vector functions are provided by transfection using a virus vector; standard methods for producing viral infection may be used.

### Packaging Functions

The packaging functions include genes for viral vector replication and packaging. Thus, for example, the packaging functions may include, as needed, functions necessary for viral gene expression, viral vector replication, rescue of the viral vector from the integrated state, viral gene expression, and packaging of the viral vector into a viral particle. The packaging functions may be supplied together or separately to the packaging cell using a genetic construct such as a plasmid or an amplicon, a Baculovirus, or HSV helper construct. The packaging functions may exist extrachromosomally within the packaging cell but are preferably integrated into the cell's chromosomal DNA. Examples include genes encoding AAV Rep and Cap proteins.

rAAV Production Systems

Numerous cell culture-based systems are known in the art for production of rAAV particles, any of which can be used to practice a method disclosed herein. The cell culture-based systems include transfection, stable cell line production, and infectious hybrid virus production systems which include Adenovirus-AAV hybrids, herpesvirus-AAV hybrids and baculovirus-AAV hybrids. rAAV production cultures for the production of rAAV virus particles all require; (1) suitable host cells, including, for example, human-derived cell lines such as HeLa, A549, or HEK293 cells and their derivatives (HEK293T cells, HEK293F cells), mammalian cell lines such as Vero, CHO cells or CHO-derived cells, or insect-derived cell lines such as SF-9 in the case of baculovirus production systems; (2) suitable helper virus function, provided by wild type or mutant adenovirus (such as temperature sensitive adenovirus), herpes virus, baculovirus, or a plasmid construct providing helper functions; (3) AAV rep and cap genes and gene products; (4) a transgene (such as a therapeutic transgene) flanked by AAV ITR sequences; and (5) suitable media and media components to support rAAV production.

A skilled artisan is aware of the numerous methods by which AAV rep and cap genes, AAV helper genes (e.g., adenovirus E1a gene, E1b gene, E4 gene, E2a gene, and VA gene), and rAAV genomes (comprising one or more genes of interest flanked by inverted terminal repeats (ITRs)) can be introduced into cells to produce or package rAAV. The phrase “adenovirus helper functions” refers to a number of viral helper genes expressed in a cell (as RNA or protein) such that the AAV grows efficiently in the cell. The skilled artisan understands that helper viruses, including adenovirus and herpes simplex virus (HSV), promote AAV replication and certain genes have been identified that provide the essential functions, e.g. the helper may induce changes to the cellular environment that facilitate such AAV gene expression and replication. In some embodiments, AAV rep and cap genes, helper genes, and rAAV genomes are introduced into cells by transfection of one or more plasmid vectors encoding the AAV rep and cap genes, helper genes, and rAAV genome. In some embodiments, AAV rep and cap genes, helper genes, and rAAV genomes can be introduced into cells by transduction with viral vectors, for example, rHSV vectors encoding the AAV rep and cap genes, helper genes, and rAAV genome. In some embodiments, one or more of AAV rep and cap genes, helper genes, and rAAV genomes are introduced into the cells by transduction with an rHSV vector. In some embodiments, the rHSV vector encodes the AAV rep and cap genes. In some embodiments, the rHSV vector encodes the helper genes. In some embodiments, the rHSV vector encodes the rAAV genome. In some embodiments, the rHSV vector encodes the AAV rep and cap genes. In some embodiments, the rHSV vector encodes the helper genes and the rAAV genome. In some embodiments, the rHSV vector encodes the helper genes and the AAV rep and cap genes.

Any suitable media known in the art may be used for the production of rAAV particles. These media include, without limitation, media produced by Hyclone Laboratories and JRH including Modified Eagle Medium (MEM), Dulbecco's Modified Eagle Medium (DMEM), and Sf-900 II SFM media as described in U.S. Pat. No. 6,723,551, which is incorporated herein by reference in its entirety. In some embodiments, the medium comprises Dynamis™ Medium, FreeStyle™ 293 Expression Medium, or Expi293™ Expression Medium from Invitrogen/ThermoFisher. In some embodiments, the medium comprises Dynamis™ Medium. In some embodiments, a method disclosed herein uses a cell culture comprising a serum-free medium, an animal-component free medium, or a chemically defined medium. In some embodiments, the medium is an animal-component free medium. In some embodiments, the medium comprises serum. In some embodiments, the medium comprises fetal bovine serum. In some embodiments, the medium is a glutamine-free medium. In some embodiments, the medium comprises glutamine. In some embodiments, the medium is supplemented with one or more of nutrients, salts, buffering agents, and additives (e.g., antifoam agent). In some embodiments, the medium is supplemented with glutamine. In some embodiments, the medium is supplemented with serum. In some embodiments, the medium is supplemented with fetal bovine serum. In some embodiments, the medium is supplemented with poloxamer, e.g., Kolliphor® P 188 Bio. In some embodiments, a medium is a base medium. In some embodiments, the medium is a feed medium.

rAAV production cultures can routinely be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, multilayer or multitray tissue culture flasks (or stacks, e.g. hyperstacks), microcarriers, and packed-bed or fluidized-bed bioreactors. rAAV vector production cultures may also include suspension-adapted host cells such as HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells and SF-9 cells which can be cultured in a variety of ways including, for example, spinner flasks, stirred tank bioreactors, and disposable systems such as the Wave bag system. Numerous suspension cultures are known in the art for production of rAAV particles, including for example, the cultures disclosed in U.S. Pat. Nos. 6,995,006, 9,783,826, and in U.S. Pat. Appl. Pub. No. 20120122155, each of which is incorporated herein by reference in its entirety.

### Packaging Cell

Any cell or cell line that is known in the art to produce rAAV particles can be used in any one of the methods disclosed herein. In some embodiments, a method of producing rAAV particles or increasing the production of rAAV particles disclosed herein uses HeLa cells, HEK293 cells, HEK293 derived cells (e.g., HEK293T cells, HEK293F cells), Vero cells, CHO cells, CHO-K1 cells, CHO derived cells, EB66 cells, BSC cells, HepG2 cells, LLC-MK cells, CV-1 cells, COS cells, MDBK cells, MDCK cells, CRFK cells, RAF cells, RK cells, TCMK-1 cells, LLCPK cells, PK15 cells, LLC-RK cells, MDOK cells, BHK cells, BHK-21 cells, NS-1 cells, MRC-5 cells, WI-38 cells, BHK cells, 3T3 cells, 293 cells, RK cells, Per.C6 cells, chicken embryo cells or SF-9 cells. In some embodiments, a method disclosed herein uses mammalian cells. In some embodiments, a method disclosed herein uses insect cells, e.g., SF-9 cells. In some embodiments, a method disclosed herein uses HEK293 cells. In some embodiments, a method disclosed herein uses HEK293 cells adapted for growth in suspension culture.

In some embodiments, a cell culture disclosed herein is a suspension culture. In some embodiments, a cell culture disclosed herein is a suspension culture comprising HEK293. In some embodiments, a cell culture disclosed herein is a suspension culture comprising HEK293 cells adapted for growth in suspension culture. In some embodiments, a cell culture disclosed herein comprises a serum-free medium, an animal-component free medium, or a chemically defined medium. In some embodiments, a cell culture disclosed herein comprises a serum-free medium. In some embodiments, suspension-adapted cells are cultured in a shaker flask, a spinner flask, a cellbag, or a bioreactor.

In some embodiments, a cell culture disclosed herein comprises cells attached to a substrate (e.g., microcarriers) that are themselves in suspension in a medium. In some embodiments, the cells are HEK293 cells.

In some embodiments, a cell culture disclosed herein is an adherent culture. In some embodiments, a cell culture disclosed herein is an adherent culture comprising HEK293. In some embodiments, a cell culture disclosed herein comprises a serum-free medium, an animal-component free medium, or a chemically defined medium. In some embodiments, a cell culture disclosed herein comprises a serum-free medium.

In some embodiments, a cell culture disclosed herein comprises a high-density cell culture. In some embodiments, the culture has a total cell density of between about 1×10E+06 cells/ml and about 30×10E+06 cells/ml. In some embodiments, more than about 50% of the cells are viable cells. In some embodiments, the cells are HeLa cells, HEK293 cells, HEK293 derived cells (e.g., HEK293T cells, HEK293F cells), Vero cells, or SF-9 cells. In further embodiments, the cells are HEK293 cells. In further embodiments, the cells are HEK293 cells adapted for growth in suspension culture.

Cell lines for use as packaging cells include insect cell lines. Any insect cell which allows for replication of AAV and which can be maintained in culture can be used in accordance with the present invention. Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. The following references are incorporated herein for their teachings concerning use of insect cells for expression of heterologous polypeptides, methods of introducing nucleic acids into such cells, and methods of maintaining such cells in culture: Methods in Molecular Biology, ed. Richard, Humana Press, N J (1995); O'Reilly et al., Baculovirus Expression Vectors: A Laboratory Manual, Oxford Univ. Press (1994); Samulski et al., 1989, J. Virol. 63:3822-3828; Kajigaya et al., 1991, Proc. Nat'l. Acad. Sci. USA 88: 4646-4650; Ruffing et al., 1992, J. Virol. 66:6922-6930; Kimbauer et al., 1996, Virol. 219:37-44; Zhao et al., 2000, Virol. 272:382-393; and Samulski et al., U.S. Pat. No. 6,204,059.

For example, virus capsids utilized in embodiments described herein can be produced using any method known in the art, e.g., by expression from a baculovirus (Brown et al., (1994) Virology 198:477-488). As a further alternative, the virus vectors of the invention can be produced in insect cells using baculovirus vectors to deliver the rep/cap genes and rAAV template as described, for example, by Urabe et al., 2002, Human Gene Therapy 13:1935-1943.

In another aspect, provided herein are methods of rAAV production in insect cells wherein a baculovirus packaging system or vectors may be constructed to carry the AAV Rep and Cap coding region by engineering these genes into the polyhedrin coding region of a baculovirus vector and producing viral recombinants by transfection into a host cell. Notably when using Baculovirus production for AAV, preferably the AAV DNA vector product is a self-complementary AAV like molecule without using mutation to the AAV ITR. This appears to be a by-product of inefficient AAV rep nicking in insect cells which results in a self-complementary DNA molecule by virtue of lack of functional Rep enzyme activity. The host cell is a baculovirus-infected cell or has introduced therein additional nucleic acid encoding baculovirus helper functions or includes these baculovirus helper functions therein. These baculovirus viruses can express the AAV components and subsequently facilitate the production of the capsids.

During production, the packaging cells generally include one or more viral vector functions along with helper functions and packaging functions sufficient to result in replication and packaging of the viral vector. These various functions may be supplied together or separately to the packaging cell using a genetic construct such as a plasmid or an amplicon, and they may exist extrachromosomally within the cell line or integrated into the cell's chromosomes.

The cells may be supplied with any one or more of the stated functions already incorporated, e.g., a cell line with one or more vector functions incorporated extrachromosomally or integrated into the cell's chromosomal DNA, a cell line with one or more packaging functions incorporated extrachromosomally or integrated into the cell's chromosomal DNA, or a cell line with helper functions incorporated extrachromosomally or integrated into the cell's chromosomal DNA.

rAAV Purification

The rAAV particles produced can be isolated using methods known in the art. In some embodiments, methods of isolating rAAV particles comprises downstream processing such as, for example, harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxylapatite chromatography, sterile filtration, or any combination(s) thereof. In some embodiments, downstream processing includes at least 2, at least 3, at least 4, at least 5 or at least 6 of harvest of a cell culture, clarification of the harvested cell culture (e.g., by centrifugation or depth filtration), tangential flow filtration, affinity chromatography, anion exchange chromatography, cation exchange chromatography, size exclusion chromatography, hydrophobic interaction chromatography, hydroxylapatite chromatography, and sterile filtration. In some embodiments, downstream processing comprises harvest of a cell culture, clarification of the harvested cell culture (e.g., by depth filtration), sterile filtration, tangential flow filtration, affinity chromatography, and anion exchange chromatography. In some embodiments, downstream processing comprises clarification of a harvested cell culture, sterile filtration, tangential flow filtration, affinity chromatography, and anion exchange chromatography. In some embodiments, downstream processing comprises clarification of a harvested cell culture by depth filtration, sterile filtration, tangential flow filtration, affinity chromatography, and anion exchange chromatography. In some embodiments, clarification of the harvested cell culture comprises sterile filtration. In some embodiments, downstream processing does not include centrifugation.

In some embodiments, a method of isolating rAAV particles comprises harvest of a cell culture, clarification of the harvested cell culture (e.g., by depth filtration), a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises harvest of a cell culture, clarification of the harvested cell culture (e.g., by depth filtration), a first sterile filtration, affinity chromatography, anion exchange chromatography (e.g., monolith anion exchange chromatography or AEX chromatography using a quaternary amine ligand), a tangential flow filtration, and a second sterile filtration. In some embodiments, a method of isolating rAAV particles comprises clarification of a harvested cell culture, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises clarification of a harvested cell culture, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, a method of isolating rAAV particles comprises clarification of a harvested cell culture by depth filtration, a first sterile filtration, a first tangential flow filtration, affinity chromatography, anion exchange chromatography (e.g., monolith anion exchange chromatography or AEX chromatography using a quaternary amine ligand), a second tangential flow filtration, and a second sterile filtration. In some embodiments, a method of isolating rAAV particles disclosed herein comprises clarification of a harvested cell culture by depth filtration, a first sterile filtration, affinity chromatography, anion exchange chromatography (e.g., monolith anion exchange chromatography or AEX chromatography using a quaternary amine ligand), tangential flow filtration, and a second sterile filtration. In some embodiments, the method does not include centrifugation. In some embodiments, clarification of the harvested cell culture comprises sterile filtration.

Recombinant AAV particles can be harvested from rAAV production cultures by harvest of the production culture comprising host cells or by harvest of the spent media from the production culture, provided the cells are cultured under conditions known in the art to cause release of rAAV particles into the media from intact host cells. Recombinant AAV particles can also be harvested from rAAV production cultures by lysis of the host cells of the production culture. Suitable methods of lysing cells are also known in the art and include for example multiple freeze/thaw cycles, sonication, microfluidization, and treatment with chemicals, such as detergents and/or proteases.

At harvest, rAAV production cultures can contain one or more of the following: (1) host cell proteins; (2) host cell DNA; (3) plasmid DNA; (4) helper virus; (5) helper virus proteins; (6) helper virus DNA; and (7) media components including, for example, serum proteins, amino acids, transferrins and other low molecular weight proteins. rAAV production cultures can further contain product-related impurities, for example, inactive vector forms, empty viral capsids, aggregated viral particles or capsids, mis-folded viral capsids, degraded viral particle.

In some embodiments, the rAAV production culture harvest is clarified to remove host cell debris. In some embodiments, the production culture harvest is clarified by filtration through a series of depth filters. Clarification can also be achieved by a variety of other standard techniques known in the art, such as, centrifugation or filtration through any cellulose acetate filter of 0.2 mm or greater pore size known in the art. In some embodiments, clarification of the harvested cell culture comprises sterile filtration. In some embodiments, the production culture harvest is clarified by centrifugation. In some embodiments, clarification of the production culture harvest does not included centrifugation.

In some embodiments, harvested cell culture is clarified using filtration. In some embodiments, clarification of the harvested cell culture comprises depth filtration. In some embodiments, clarification of the harvested cell culture further comprises depth filtration and sterile filtration. In some embodiments, harvested cell culture is clarified using a filter train comprising one or more different filtration media. In some embodiments, the filter train comprises a depth filtration media. In some embodiments, the filter train comprises one or more depth filtration media. In some embodiments, the filter train comprises two depth filtration media. In some embodiments, the filter train comprises a sterile filtration media. In some embodiments, the filter train comprises 2 depth filtration media and a sterile filtration media. In some embodiments, the depth filter media is a porous depth filter. In some embodiments, the filter train comprises Clarisolve® 20MS, Millistak+® COHC, and a sterilizing grade filter media. In some embodiments, the filter train comprises Clarisolve® 20MS, Millistak+® COHC, and Sartopore® 2 XLG 0.2 pm. In some embodiments, the harvested cell culture is pretreated before contacting it with the depth filter. In some embodiments, the pretreating comprises adding a salt to the harvested cell culture. In some embodiments, the pretreating comprises adding a chemical flocculent to the harvested cell culture. In some embodiments, the harvested cell culture is not pre-treated before contacting it with the depth filter.

In some embodiments, the clarified feed is concentrated via tangential flow filtration (“TFF”) before being applied to a chromatographic medium, for example, affinity chromatography medium. Large scale concentration of viruses using TFF ultrafiltration has been described by Paul et al, Human Gene Therapy 4:609-615 (1993). TFF concentration of the clarified feed enables a technically manageable volume of clarified feed to be subjected to chromatography and allows for more reasonable sizing of columns without the need for lengthy recirculation times. In some embodiments, the clarified feed is concentrated between at least two-fold and at least ten-fold. In some embodiments, the clarified feed is concentrated between at least ten-fold and at least twenty-fold. In some embodiments, the clarified feed is concentrated between at least twenty-fold and at least fifty-fold. In some embodiments, the clarified feed is concentrated about twenty-fold. One of ordinary skill in the art will also recognize that TFF can also be used to remove small molecule impurities (e.g., cell culture contaminants comprising media components, serum albumin, or other serum proteins) form the clarified feed via diafiltration. In some embodiments, the clarified feed is subjected to diafiltration to remove small molecule impurities. In some embodiments, the diafiltration comprises the use of between about 3 and about 10 diafiltration volume of buffer. In some embodiments, the diafiltration comprises the use of about 5 diafiltration volume of buffer. One of ordinary skill in the art will also recognize that TFF can also be used at any step in the purification process where it is desirable to exchange buffers before performing the next step in the purification process. In some embodiments, the methods for isolating rAAV from the clarified feed disclosed herein comprise the use of TFF to exchange buffers.

Affinity chromatography can be used to isolate rAAV particles from a composition. In some embodiments, affinity chromatography is used to isolate rAAV particles from the clarified feed. In some embodiments, affinity chromatography is used to isolate rAAV particles from the clarified feed that has been subjected to tangential flow filtration. Suitable affinity chromatography media are known in the art and include without limitation, AVB Sepharose™ POROS™ CaptureSelect™ AAVX affinity resin, POROS™ CaptureSelect™ AAV9 affinity resin, and POROS™ CaptureSelect™ AAV8 affinity resin. In some embodiments, the affinity chromatography media is POROS™ CaptureSelect™ AAV9 affinity resin. In some embodiments, the affinity chromatography media is POROS™ CaptureSelect™ AAV8 affinity resin. In some embodiments, the affinity chromatography media is POROS™ CaptureSelect™ AAVX affinity resin.

Anion exchange chromatography can be used to isolate rAAV particles from a composition. In some embodiments, anion exchange chromatography is used after affinity chromatography as a final concentration and polish step. Suitable anion exchange chromatography media are known in the art and include without limitation, Unosphere Q (Biorad, Hercules, Calif), and N-charged amino or imino resins such as e.g., POROS 50 PI, or any DEAE, TMAE, tertiary or quaternary amine, or PEI-based resins known in the art (U.S. Pat. No. 6,989,264; Brument et al., Mol. Therapy 6(5):678-686 (2002); Gao et al., Hum. Gene Therapy 11:2079-2091 (2000)). In some embodiments, the anion exchange chromatography media comprises a quaternary amine. In some embodiments, the anion exchange media is a monolith anion exchange chromatography resin. In some embodiments, the monolith anion exchange chromatography media comprises glycidylmethacrylate-ethylenedimethacrylate or styrene-divinylbenzene polymers. In some embodiments, the monolith anion exchange chromatography media is selected from the group consisting of CIMmultus™ QA-1 Advanced Composite Column (Quaternary amine), CIMmultus™ DEAE-1 Advanced Composite Column (Diethylamino), CIM® QA Disk (Quaternary amine), CIM® DEAE, and CIM® EDA Disk (Ethylene diamino). In some embodiments, the monolith anion exchange chromatography media is CIMmultus™ QA-1 Advanced Composite Column (Quaternary amine). In some embodiments, the monolith anion exchange chromatography media is CIM® QA Disk (Quaternary amine). In some embodiments, the anion exchange chromatography media is CIM QA (BIA Separations, Slovenia). In some embodiments, the anion exchange chromatography media is BIA CIM® QA-80 (Column volume is 80 mL). One of ordinary skill in the art can appreciate that wash buffers of suitable ionic strength can be identified such that the rAAV remains bound to the resin while impurities, including without limitation impurities which may be introduced by upstream purification steps are stripped away.

In additional embodiments the disclosure provides compositions comprising isolated rAAV particles produced according to a method disclosed herein. In some embodiment, the composition is a pharmaceutical composition comprising a pharmaceutically acceptable carrier.

As used herein the term “pharmaceutically acceptable means a biologically acceptable formulation, gaseous, liquid or solid, or mixture thereof, which is suitable for one or more routes of administration, in vivo delivery or contact. A “pharmaceutically acceptable” composition is a material that is not biologically or otherwise undesirable, e.g., the material may be administered to a subject without causing substantial undesirable biological effects. Thus, such a pharmaceutical composition may be used, for example in administering rAAV isolated according to the disclosed methods to a subject. Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions. Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Pharmaceutical compositions and delivery systems appropriate for rAAV particles and methods and uses of the invention are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, Md.; and Poznansky et al, Drug Delivery Systems (1980), R. L. Juliano, ed., Oxford, N.Y., pp. 253-315).

As used herein, the rAAVs described can be used as a gene therapy to treat a MECP2 deficiency. Methods of treatment includes injecting said rAAV in a subject requiring it. One skilled in the art would understand the quantities needed to treat the subject, as it would depend on multiple factors including size, age, and gender of the subject.

### Methods of Treatment

In another aspect, methods of treatment are presented. The method comprises administering an effective amount of the pharmaceutical composition comprising any of the desired constructs or rAAV virions described above to a patient in need thereof.

In some embodiments, the effective amount is at least 1×10⁸ viral genomes per dose. In some embodiments, the effective amount is at least 5×10⁸ viral genomes/dose, 7.5×10⁸ viral genomes/dose, at least 1×10⁹ viral genomes/dose, at least 2.5×10⁹ viral genomes/dose, at least 5×10⁹ viral genomes/dose.

In some embodiments, the effective amount is at least 1×10¹¹ viral genomes/kg patient weight, at least 5×10¹¹ viral genomes/kg, at least 1×10¹² viral genomes/kg, at least 5×10¹² viral genomes/kg, at least 1×10¹³ viral genomes/kg, at least 1×10¹⁴ viral genomes/kg, or at least 5×10¹⁴. In some embodiments, the rAAV is dosed based upon brain weight rather than by bodyweight. In some embodiments, the rAAV dose is considered a low dose and is particularly beneficial for a CNS indication.

In some embodiments, the rAAV is administered intravenously. In some embodiments, the rAAV is administered intrathecally. In some embodiments, the rAAV is administered by intracerebral ventricular injection. In some embodiments, the rAAV is administered by intracisternal magna administration. In some embodiments, the rAAV is administered by intravitreal injection.

In various embodiments a method of treating a MECP2-associated disorder (Rett Syndrome) in a subject is disclosed, wherein the method comprises administering to the subject an effective amount of any of the polynucleotide constructs described herein, or the vectors, or the rAAV comprising the vectors, or the virion, or any pharmaceutical composition comprising any of these elements, as described herein.

In certain embodiments, as an additional modification of the therapeutic cassette, the gene of interest (GOI) was tested as codon optimized and wild type. It was determined that several different codon-optimized MECP2 sequences were compared with the wild-type human MECP2 sequence (SEQ ID NO: 7) and it was determined that codon optimized MECP2 expression was not improved over wild-type MECP2 sequence (SEQ ID NO: 7). Thus, in the lead therapeutic cassettes, wild-type human MECP2 codon is utilized.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### Example 1 Optimizing MECP2 Expression Cassette

FIG. 1A shows the MeCP2 dosage sensitive gene therapy cassettes designed to reduce dosage sensitivity, prevent overexpression and achieve a therapeutic setpoint transgene level. FIG. 1B shows graphs of flow cytometry data illustrating the effects of different modifications of the therapeutic cassette (feed forward circuit) for tuning MeCP2 protein expression level. Reporter constructs, in which the reporter mNeonGreen is fused to hMeCP2 and a second expression cassette allowing mRuby to be measured as a transfection control, were transfected into HEK cells and after 48 hrs cells were processed, analyzed by flow cytometry and levels of mRuby (transfection efficiency) and mNeonGreen (MeCP2) were measured.

FIG. 2 is a schematic showing exemplifications of the polynucleotide cassette elements that are modulated to adjust dosage insensitivity and setpoint of expression of MeCP2.

### Example 2 Mouse Model Data Illustrating Efficacy Using Therapeutic MECP2 Cassettes

Efficacy: To demonstrate efficacy in vivo, either regulated or unregulated MECP2 constructs were delivered to male Mecp2 knockout mice using AAV. EXACT-regulated MECP2 constructs packaged into AAV9 showed highly efficient viral genome packaging. This severe mouse model displays reduced lifespan (median survival ˜11 weeks) and prominent respiratory and motor impairments. Mice dosed by neonatal injection (ICV) with 1e11 vg/mouse of the lead regulated MECP2 construct showed significant improvement in survival (median survival extension=14 weeks weeks) and a concomitant improvement in RTT-like clinical score. In contrast, mice dosed with the unregulated constructs did not show any improvement in survival, possibly due to overexpression toxicity. At a higher dose of 3×10¹¹ vg/mouse, mice treated with the regulated lead construct showed a profound improvement in lifespan (75% survival beyond 35 weeks) and significant amelioration of RTT-like phenotypes. At this higher dose, mice treated with the unregulated construct showed severe signs of MeCP2 overexpression and were euthanised at ˜3 weeks. These data demonstrate the ability of the EXACT circuit to enable strong efficacy and significantly improve the safety profile of an MECP2 gene therapy vector. In separate toxicity studies, performed by Labcorp, safety was demonstrated in non-human primates treated at therapeutically relevant doses. Finally, the safety of the non-mammalian miRNA element was also assessed in human cell lines by RNAseq, which showed no changes in the expression of the top predicted human gene targets when transfected with the miRNA.

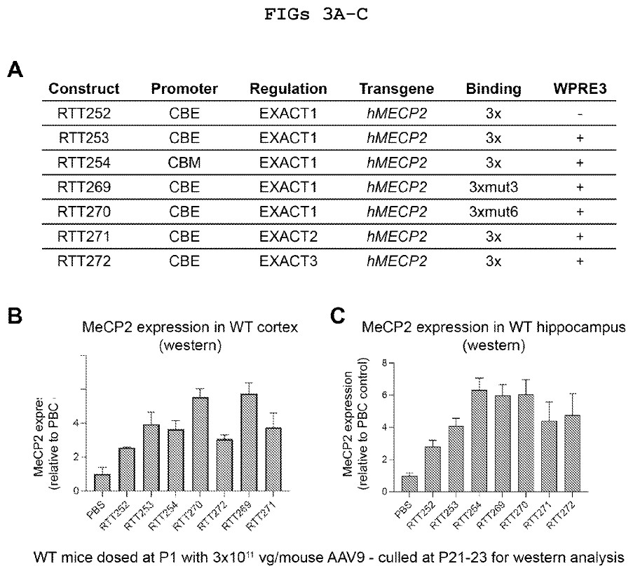

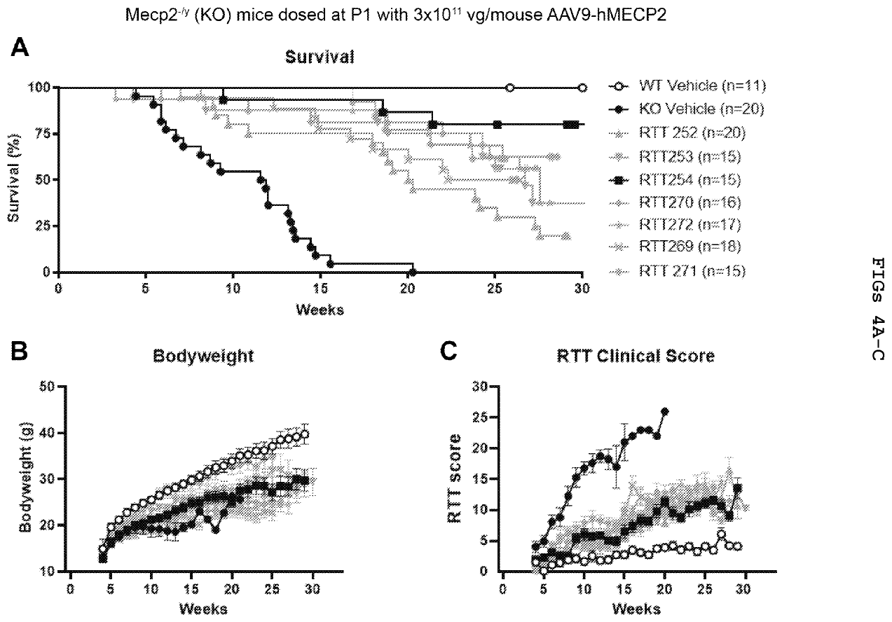

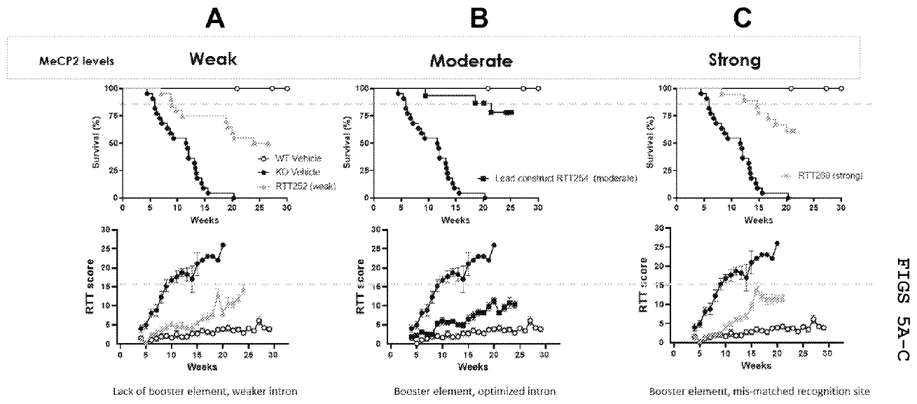

These results are illustrated in FIGS. 3A-C which depict the modular polynucleotide sequence elements and design strategy for the MeCP2 constructs (FIG. 3A) along with MeCP2 expression data. 21-23 days after dosing wild-type mice with AAV9-RTT252, AAV9-RTT253, AAV9-RTT254 (also referred to as NGN-401), AAV9-RTT269, AAV9-RTT270, AAV9-RTT271 or AAV9-RTT272, tissue samples were collected and analyzed by western blot to determine levels of MeCP2 expression in WT cortex (FIG. 3B) and WT hippocampus (FIG. 3C).

FIGS. 4A-C are graphic depictions comparing the therapeutic MEPC2 constructs for survival (FIG. 4A), bodyweight (FIG. 4B), and RTT clinical score (FIG. 4C) in Mecp2^{-/y} (KO) mice following injection at P1 with 3×10¹¹ vg/mouse of a therapeutic AAV9-MECP2 construct. The RTT clinical score is an observational scoring system used to determine the severity of the Rett phenotype in mice. Scoring ranges from 0 (like wild-type) to 5 (most severe) for each individual component of the phenotype.

FIGS. 5A-C depict the systematic tuning using different polynucleotide cassette components to identify and titrate expression levels to obtain optimal efficacy-which is an intermediate or moderate level of expression. Survival plots and RTT clinical scores are shown for Mecp2^{-/y} animals dosed with 3×10¹¹ vg/mouse of an AAV9-MECP2 construct expressing weak (FIG. 5A), moderate (FIG. 5B) or strong (FIG. 5C) levels of transgenic MeCP2.

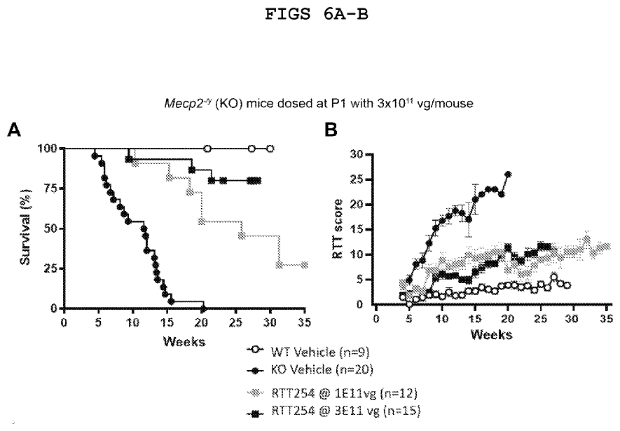

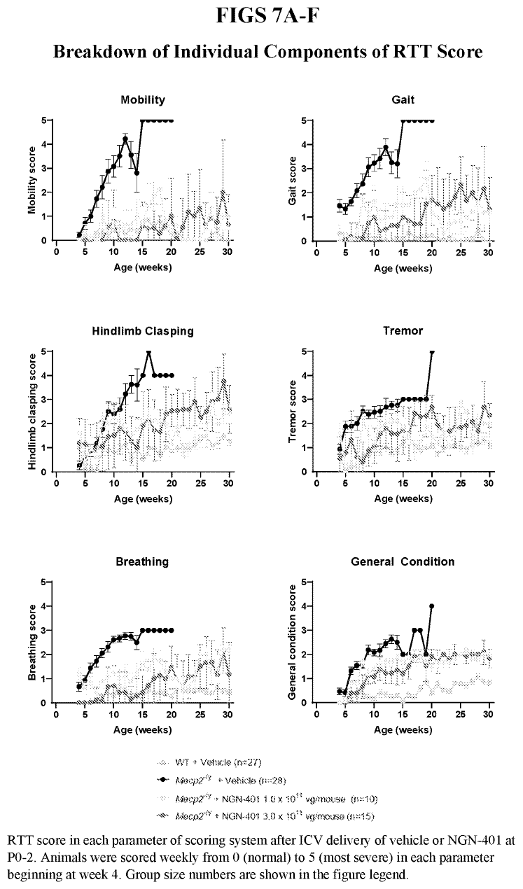

FIGS. 6A-B depict the improvement in survival (FIG. 6A) and efficacy (RTT phenotype score, (FIG. 6B) for AAV9-RTT254 treated KO animals compared with vehicle-treated KO animals.

FIGS. 7A-F depict the graphic results of improved motor and breathing phenotype domains in AAV9-RTT254 treated KO mice compared to controls, at two doses (1×10¹¹ vg and 3×10¹¹ vg).

### Example 3: Dose Selection in Hemizygous Male Mouse Model: RTT254/NGN-401 Survival & Scoring Data to 52 Weeks Post-Injection

The hemizygous male mouse model of Rett syndrome (RTT) has a complete knockout of Mecp2 in every cell (Mecp2^{-/y}), yielding rapid development of robust and reproducible RTT-like phenotypes, such as breathing disturbances, debilitating apnea events, spasticity, motor incoordination, and loss of ambulation. The mice typically only survive to 5-20 weeks of age, with a median survival of approximately 10 weeks.

An in vivo efficacy study was conducted to test NGN-401 in the male Mecp2^{-/y} mouse model. Mecp2^{-/y} mice were dosed with NGN-401 or vehicle via intracerebroventricular (ICV) injection at postnatal day P0-2. NGN-401 was administered at a dose level of either 1.0×10¹¹ or 3.0×10¹¹ total vg/mouse, which was selected based on early proof-of-concept in vivo studies. Mice dosed with NGN-401 were followed to track survival and disease phenotypes.

A total of 10-29 animals per group were included in the study to assess survival and RTT phenotype amelioration. RTT phenotypes were assessed using a scoring system developed at the University of Edinburgh. Animals treated with NGN-401 showed a marked increase in survival (median survival was extended from 9 weeks in vehicle control Mecp2^{-/y} mice to 23 and 37 weeks at doses of 1.0×10¹¹ and 3.0×10¹¹ vg/mouse of NGN-401, respectively). Amelioration of the RTT phenotype was also observed, with reduced RTT-like phenotypes compared to vehicle treated mice. Greater efficacy was observed at the higher dose of NGN-401.

TABLE 1
Study Design

| | | | | | Number |
|---|---|---|---|---|---|
| | | | | Dose | (Necropsy/ |
| Group | Sex | Genotype | Test Article | (vg/mouse) | Survival |
| 1 | M | WT | Vehicle | — | 6/27 |
| 2 | M | Mecp2^{-/y} | Vehicle | — | 6/28 |
| 3 | M | Mecp2^{-/y} | NGN-401 | 1.0 × 10¹¹ | 6/10 |
| 4 | M | Mecp2^{-/y} | NGN-401 | 3.0 × 10¹¹ | 6/15 |

All clinical assessments were carried out by personnel blind to both genotype and treatment. An animal caretaker conducted daily cage side observations for each animal and abnormal findings were recorded. Body weight was recorded once a week from P28 onwards. Individual body weights were measured using a countertop scale. RTT phenotypes were assessed weekly from P28 onwards using the RTT score, a non-invasive observational scoring system; a modified version of the scoring system developed by Dr. Jacky Guy, University of Edinburgh (Guy et al., 2007 (DOI: 10.1126/science.1138389)). Animals were given a score of 0-5 by a blinded researcher in each of 6 parameters: mobility, gait, hindlimb clasping, tremor, breathing, and general condition. A score of 0 signifies the phenotype of wild-type animals, and a score of 5 represents the most severe phenotype. These scores are then combined to give an aggregate RTT phenotype score. Detailed records for each animal were collected. Animals were culled when they reached the humane endpoint criteria for euthanasia, or the planned terminal sacrifice at 30 weeks. Due to the extended survival in Mecp2^{-/y} mice treated with NGN-401, these cohorts were extended out to 52 weeks to fully assess survival and phenotype improvements.

### In Life Evaluations: Survival

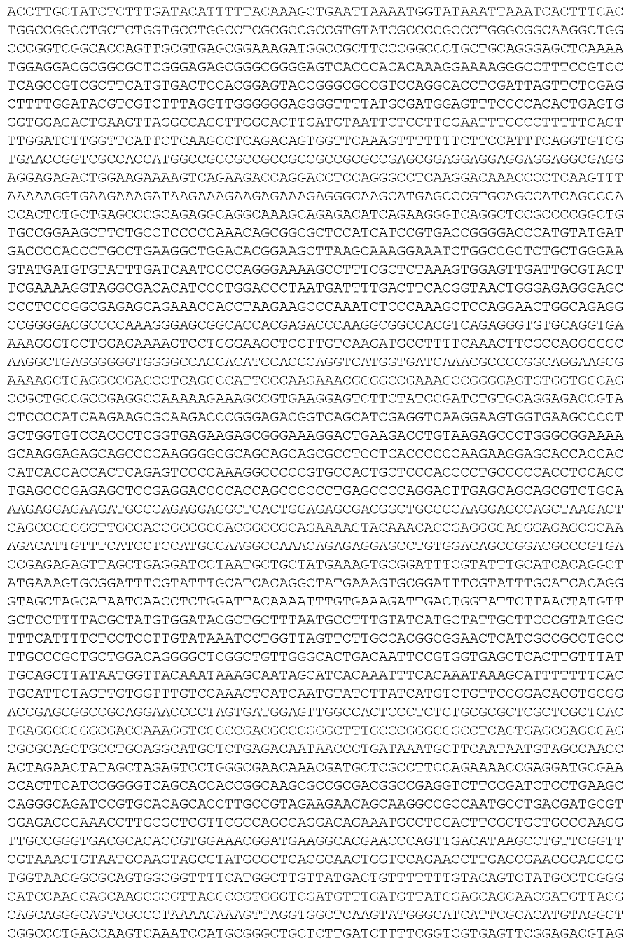

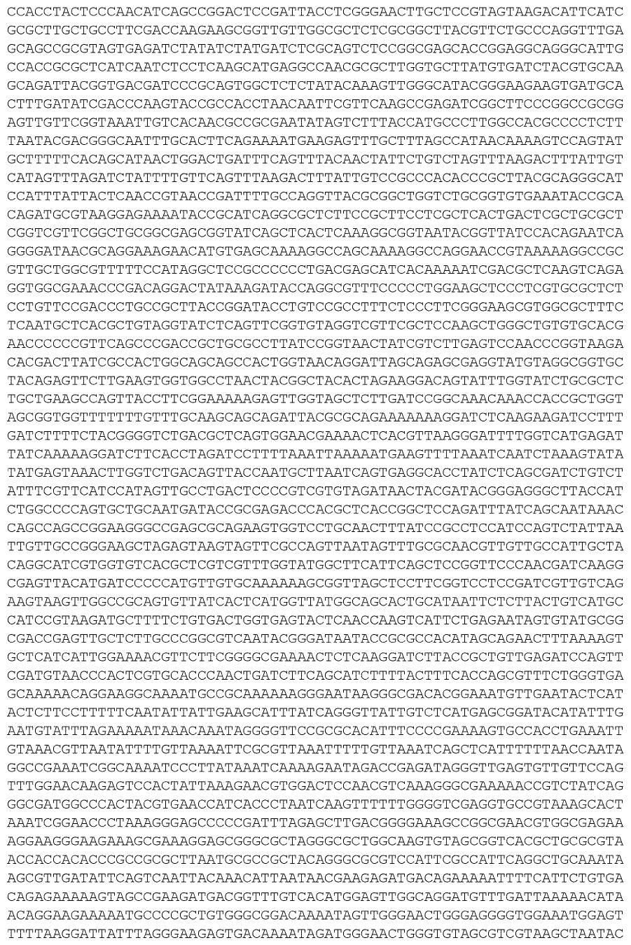

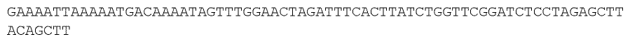

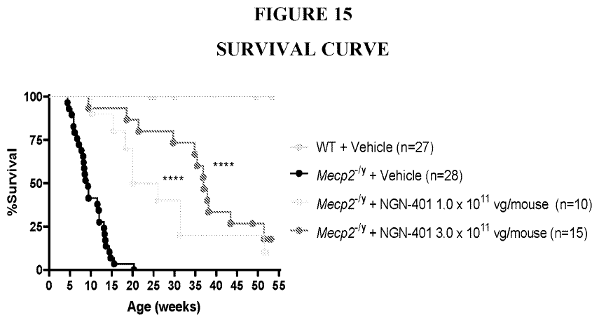

Mice treated with NGN-401 showed a significant increase in survival compared to vehicle treated mice (FIG. 15). Median survival was extended from 9 weeks in vehicle control Mecp2^{-/y} mice to 23 weeks at a dose of 1.0×10¹¹ vg/mouse, and 37 weeks at a dose of 3.0×10¹¹ vg/mouse (p<0.0001, Mantel-Cox test). All mice in the vehicle treated cohort were found dead or reached a humane endpoint by week 20 of the study. In contrast, the longest-lived mice reached 52 weeks of age at both doses of NGN-401 treatment, at which point the study was ended. Survival curve for animals found dead or reaching humane endpoint with RTT-like phenotypes after ICV delivery of vehicle or NGN-401 at P0-2. Pups found missing before weaning or mice culled for reasons unrelated to RTT phenotype were not included. Group size numbers are shown in the figure legend. **** p<0.0001 Mantel-Cox test.

### In Life Evaluations: Bodyweight

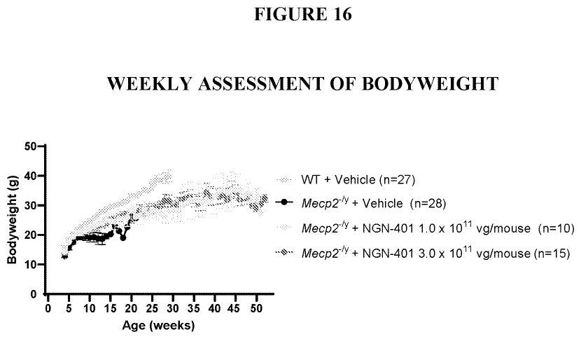

Bodyweights were recorded weekly for each animal from P28 onwards. There was a pronounced difference in bodyweight between vehicle treated WT and vehicle treated Mecp2^{-/y} mice, with significant differences between groups from 8 weeks through to 13 weeks of age (Mixed-effects model (REML) with Sidak's multiple comparisons test) (FIG. 16). When compared to vehicle treated Mecp2^{-/y} mice, Mecp2^{-/y} mice treated with NGN-401 at a dose of either 1.0×10¹¹ or 3.0×10¹¹ vg/mouse did not show significant differences in bodyweight (Mixed-effects model (REML) with Dunnett's multiple comparisons test). NGN-401 treated Mecp2^{-/y} mice continued to gain bodyweight throughout the study.

In-life bodyweight following ICV delivery of vehicle or NGN-401 at P0-2. Animals were weighed weekly beginning at P28. Group size numbers are shown in the figure legend.

### In Life Evaluations: RTT Phenotype Score

Male Mecp2^{-/y} mice develop a rapidly progressing RTT-like phenotype from −4 weeks of age when they develop overt locomotor, autonomic and breathing disturbances. Mice were assessed weekly for RTT-like phenotypes from P28 onwards using an observational scoring system. The aggregate RTT score reflects the summation of individual scores from all six parameters evaluated (mobility, gait, hindlimb clasping, tremor, breathing and general condition).

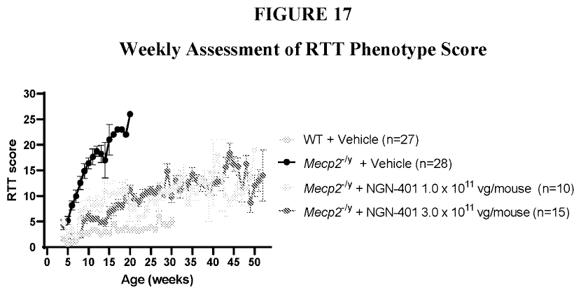

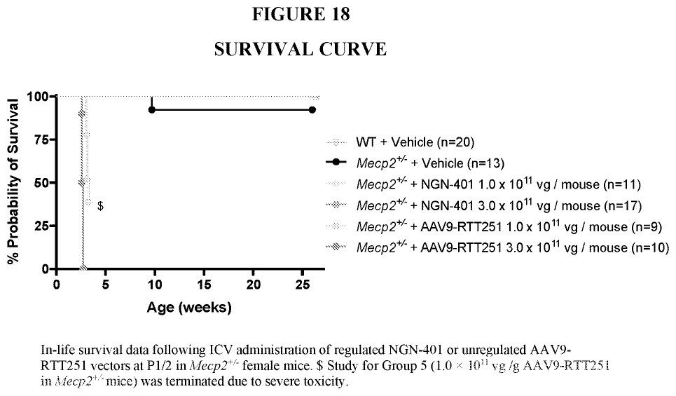

There was a pronounced difference in aggregate RTT score between the vehicle treated WT and vehicle treated Mecp2^{-/y} mice throughout the study with significant differences between groups from 5 weeks through to 13 weeks of age (Mixed-effects model (REML) with Sidak's multiple comparisons test) (FIG. 17). Similarly, when compared to vehicle treated Mecp2^{-/y} mice, Mecp2^{-/y} mice treated with NGN-401 at a dose of either 1.0×10¹¹ or 3.0×10¹¹ vg/mouse showed a robust improvement in phenotype score throughout the study, with significant differences between NGN-401 treated and vehicle treated mice from 5 weeks through to 13 weeks of age (Mixed-effects model (REML) with Dunnett's multiple comparisons test). Statistical comparisons could not be performed past 13 weeks of age due to insufficient mice remaining in the vehicle treated Mecp2^{-/y} cohort.

Combined RTT phenotype score after ICV delivery of vehicle or NGN-401 at P0-2. Animals were scored weekly from 0 (normal) to 5 (most severe) in each parameter, beginning at P28. Scores were combined to give an aggregate RTT phenotype score. Group size numbers are shown in the figure legend (FIG. 17).

### In Life Evaluations: Individual RTT Phenotypes

NGN-401 treated animals showed a marked improvement in the phenotypic score. As shown in FIG. 7A-F, analysis of each of the six RTT parameters evaluated (i.e., mobility, gait, hindlimb clasping, tremor, breathing, and general condition) showed that ICV delivery of NGN-401 led to a reduction in all parameters, particularly mobility, gait, and breathing. Animals treated with vehicle developed a rapid rise in clinical score to highest level (score=5) in mobility, gait and hindlimb clasping parameters, exhibiting a very severe phenotype of loss of locomotion, and clasping in both hindlimbs. This rapid rise in clinical score was prevented with NGN-401 treatment. Similarly, in the breathing parameter, NGN-401 treatment reduced the frequency of visible apneas.

### Example 4

### Tolerability in Heterozygous Female Mouse Model: Survival & Scoring to 25 Weeks Post-Injection

The female heterozygous Mecp2^{+/-} mouse model exhibits mild and variable phenotypes, rendering it undesirable as a robust efficacy model. However, the sex, genotype, and mosaicism of MeCP2 expression in these animals is more representative of the female Rett syndrome (RTT) patient.

An in vivo study in female heterozygous mice was performed to assess the tolerability of NGN-401, an MECP2 gene therapy construct in which expression levels are regulated by an EXACT miRNA circuit. NGN-401 was compared to AAV9-RTT251, an unregulated MECP2 vector which does not contain the EXACT miRNA regulatory circuit. The vectors were manufactured using a baculovirus production system and administered via intracerebroventricular (ICV) injection at P1/P2 at a dose of either 1.0×10¹¹ vg/mouse or 3.0×10¹¹ vg/mouse.

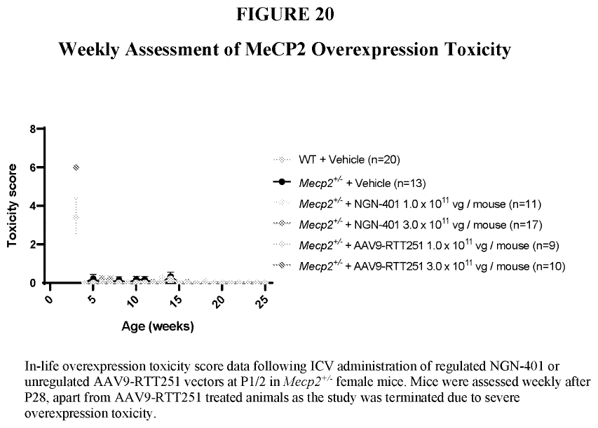

A total of 9-20 animals per group were injected and the tolerability of NGN-401 was assessed using an MeCP2 overexpression toxicity scoring system developed at the University of Edinburgh. As shown in FIG. 20, animals treated with NGN-401 showed no evidence of toxicity during the 26 week in-life portion of the study. In contrast, mice treated with an unregulated MECP2 vector (AAV9-RTT251) showed severe toxicity at approximately 3 weeks of age, leading to death or euthanasia for humane purposes.

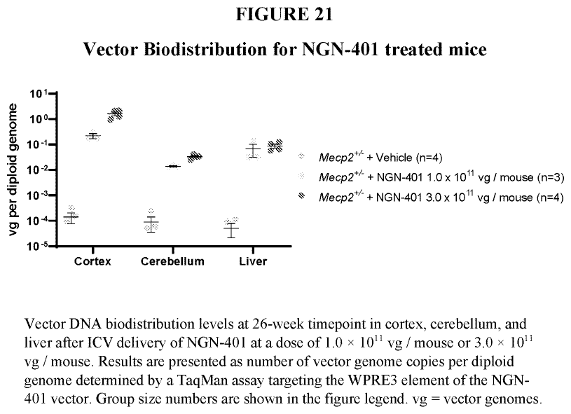

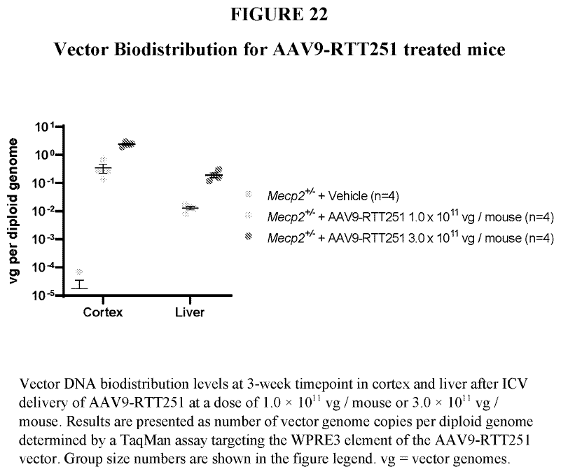

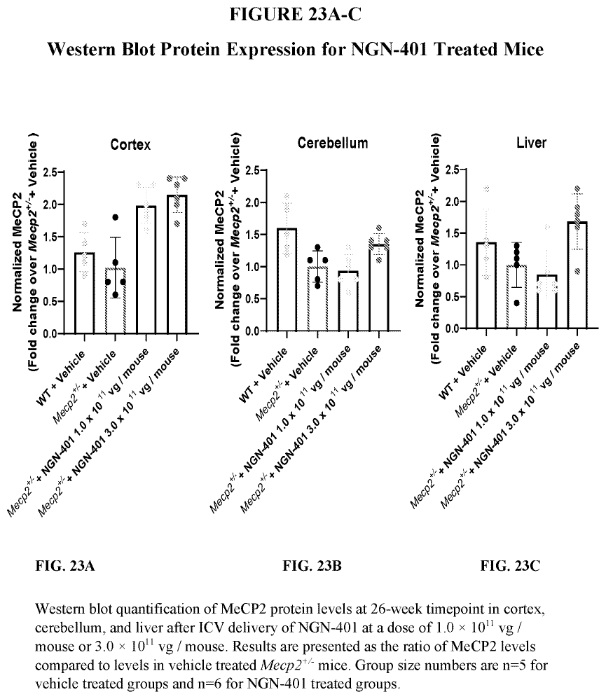

As shown in FIGS. 21-22, vector DNA biodistribution analysis and western blot analysis showed that mice treated with either NGN-401 or AAV9-RTT251 exhibited similar levels of vector genome copies at equivalent doses. However, as shown in FIGS. 23A-C-24A-B, MeCP2 expression levels varied dramatically. Mice treated with NGN-401 expressed vector derived MeCP2 protein at a maximum of 120% of the endogenous protein levels of the Mecp2^{+/-} mouse, whereas vector derived MeCP2 protein levels in AAV9-RTT251 treated mice reached up to 1,900% of the endogenous protein levels of the Mecp2^{+/-} mouse. In conclusion, NGN-401 is well-tolerated in a heterozygous Mecp2^{+/-} mouse model that closely mimics the relevant patient population and overcomes the severe toxicity observed with an equivalent, unregulated vector.

TABLE 2
Study Design

| | | | | Dose | Survival |
|---|---|---|---|---|---|
| Group | Sex | Genotype | Test Article | (vg/mouse) | (Actual enrolled) |
| 1 | F | WT | Vehicle | — | 20 |
| 2 | F | Mecp2^{+/-} | Vehicle | — | 13 |
| 3 | F | Mecp2^{+/-} | NGN-401 | 1.0 × 10¹¹ | 11 |
| 4 | F | Mecp2^{+/-} | NGN-401 | 3.0 × 10¹¹ | 17 |
| 5 | F | Mecp2^{+/-} | AAV9-RTT251 | 1.0 × 10¹¹ | 9 |
| 6 | F | Mecp2^{+/-} | AAV9-RTT251 | 3.0 × 10¹¹ | 10 |

All clinical assessments were carried out by personnel blind to both genotype and treatment. An animal caretaker conducted daily cage side observations for each animal and abnormal findings were flagged. For mice treated with NGN-401, body weight was recorded once a week from P28 onwards. Mice treated with AAV9-RTT251 died or reached a humane endpoint before P28, so bodyweights were not obtained. For mice treated with NGN-401, MeCP2 overexpression toxicity was assessed weekly from P28 onwards using a 6-point scoring system developed by Kamal Gadalla at the University of Edinburgh. Mice treated with AAV9-RTT251 died or reached a humane endpoint before planned initiation of toxicity scoring. Mice that reached humane endpoint were scored for MeCP2 overexpression toxicity before culling.

### In Life Evaluations: Survival

In-life safety was monitored for 26 weeks. WT mice and Mecp2^{+/-} mice treated with NGN-401 at either 1.0×10¹¹ vg/mouse or 3.0×10¹¹ vg/mouse did not suffer any spontaneous deaths and no animals had to be culled due to reaching a humane endpoint. In contrast, all Mecp2^{+/-} mice treated with AAV9-RTT251 at 3.0×10¹¹ vg/mouse were found dead or reached a humane endpoint by P19. At the lower dose of 1.0×10¹¹ vg/mouse, over half of the mice treated with AAV9-RTT251 were found dead or had reached a humane endpoint by P23, at which point this arm of the study was stopped for ethical reasons and the remaining mice culled. For vehicle treated Mecp2^{+/-} mice, one animal was culled due to reaching a humane endpoint at approximately 10 weeks of age. All other vehicle treated Mecp2^{+/-} mice survived until the end of the 26-week study (Error! Reference source not found.).

### In Life Evaluations: Body Weight

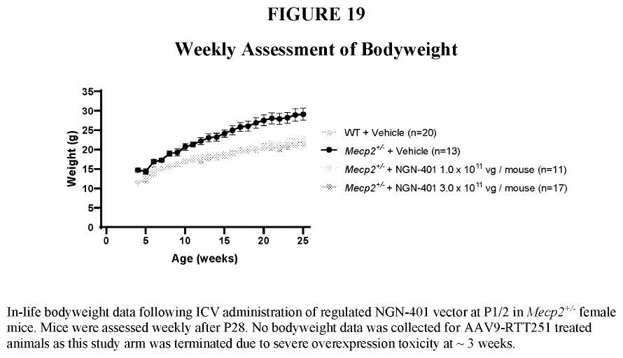

For NGN-401 and vehicle treated mice, bodyweights were recorded weekly from P28 onwards. A robust phenotype effect was observed. Vehicle-treated Mecp2^{+/-} bodyweight was increased compared to vehicle treated WT animals (at 26 weeks, mean bodyweight of vehicle-treated Mecp2^{-/-} mice was 29.1 g, compared to 21.2 g for vehicle-treated WT animals). In contrast, Mecp2^{+/-} mice treated with NGN-401 showed a correction of bodyweight to WT levels. For AAV9-RTT251 treated mice, bodyweight was not measured due to mice found dead or reaching a humane endpoint before initiation of phenotyping (FIG. 19).

### In Life Evaluations: MeCP2 Overexpression Toxicity Score

Toxicity phenotypes were monitored using a scoring system developed by Dr. Kamal Gadalla at the University of Edinburgh to classify deleterious effects associated with MeCP2 overexpression. Severe toxicity was observed in both unregulated AAV9-RTT251 treatment groups. For animals treated with the highest dose of 3.0×10¹¹ vg/mouse, all mice were either found dead or had reached the maximum toxicity score of six by P19 and were culled for humane reasons. For animals treated with the lower dose of 1.0×10¹¹ vg/mouse, over half of the mice were found dead or reached the maximum toxicity score of six by P23. At this point the AAV9-RTT251 arm of the study was terminated for ethical reasons. In contrast, animals treated with NGN-401 at equivalent doses showed no observable in-life toxicity and maintained an average toxicity score near 0 through 26 weeks of age (Error! Reference source not found.20).

### Vector Biodistribution

A TaqMan qPCR assay targeting the WPRE3 component of the NGN-401 and AAV9-RTT251 vectors was used to determine the levels of vector DNA across various regions. For NGN-401 treated mice (Error! Reference source not found.), biodistribution was measured at 26 weeks of age, at the end of the in-life portion of the study. Vector genome presence was detected in a dose-dependent manner in the cortex, cerebellum, and liver in all NGN-401 treated animals, with levels being highest in the cortex and lowest in the cerebellum. For vehicle treated mice, some background signal was amplified in individual samples, suggestive of trace levels of vector DNA that may have been introduced during tissue collection or nucleic acid extraction, however it was typically below the assay limit of quantification.

For AAV9-RTT251 treated mice, biodistribution was measured at approximately 3 weeks of age, at which point mice had to be culled having reached a humane endpoint due to overexpression toxicity. Vector genome presence was detected in a dose-dependent manner in the cortex and liver in all AAV9-RTT251 treated animals (FIG. 22). Vector genome levels in the cortex were broadly similar for NGN-401 and AAV9-RTT251 at equivalent doses. This shows that the prevention of toxicity in NGN-401 was related to the EXACT regulation of expression levels and not due to differences in vector biodistribution.

### MeCP2 Expression: Western Blot Data

Western blot analysis using an anti-MeCP2 antibody was used to measure protein expression across various regions. The anti-MeCP2 antibody recognizes both mouse and human versions of the protein, therefore measured protein levels were a combination of mouse endogenous MeCP2 protein and vector derived human protein. Results showed that while mice treated with either NGN-401 or AAV9-RTT251 exhibited similar levels of vector genome copies at equivalent doses, MeCP2 expression levels varied dramatically. In the cortex, which had the highest levels of vector biodistribution, mice treated with NGN-401 (Error! Reference source not found.23A) expressed vector derived MeCP2 protein at 98% and 115% of levels in vehicle treated Mecp2^{+/+} at doses of 1.0×10¹¹ and 3.0×10¹¹ vg/mouse, respectively. In the cerebellum, levels were lower, with no detectable vector derived protein at the lower dose and 35% of levels in vehicle treated Mecp2^{+/-} at the higher dose (FIG. 23B). Crucially, even at the highest dose in the well transduced cortex, treatment with NGN-401 led to overall MeCP2 protein levels only 70% above levels in vehicle treated WT mice. This demonstrates the ability of NGN-401 to maintain protein expression within physiological limits.

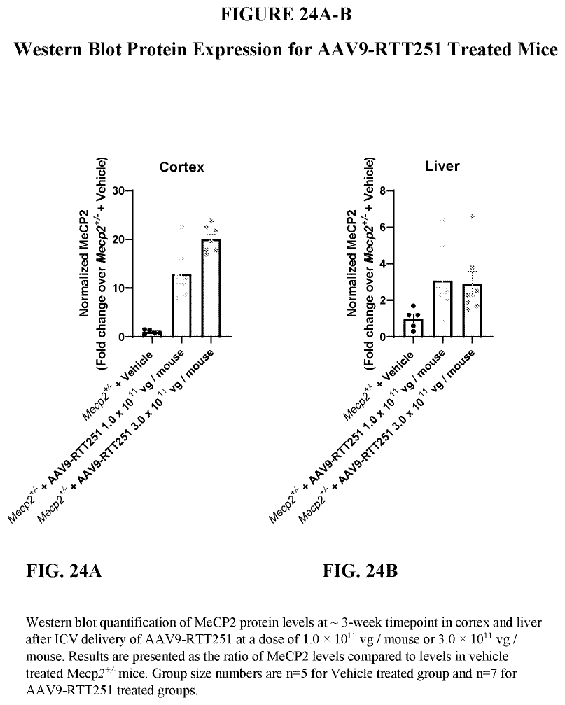

In contrast, mice treated with AAV9-RTT251 (Error! Reference source not found.24A) expressed vector derived MeCP2 protein in the cortex at 1,200% and 1,900% of levels in vehicle treated Mecp2^{+/-} mice for doses of 1.0×10¹¹ and 3.0×10¹¹ vg/mouse, respectively. Differences in the liver were less marked (FIG. 24B), with NGN-401 treated mice expressing vector derived MeCP2 protein at around 70% of endogenous MeCP2 levels of Mecp2^{+/-} mice at the highest dose compared to 190% in AAV9-RTT251 treated mice. This demonstrated that in the absence of the regulatory circuit present in NGN-401, vector derived MeCP2 protein levels are dramatically higher than normal physiological levels.

### Example 5

### Maximum Feasible Dose in Heterozygous Female Mouse Model: Survival & Scoring to 8 Weeks Post-Injection, Plus Expression (Western) & Histopathology

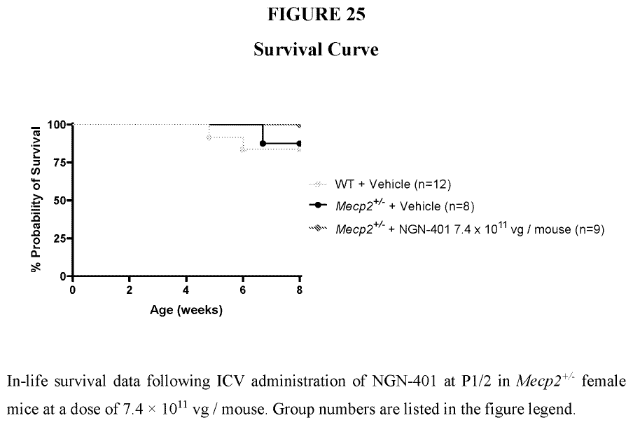

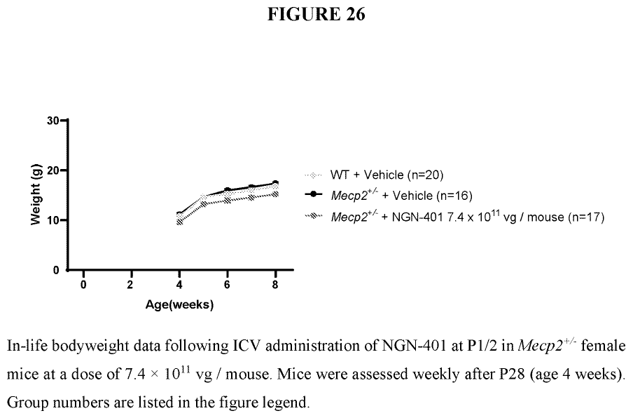

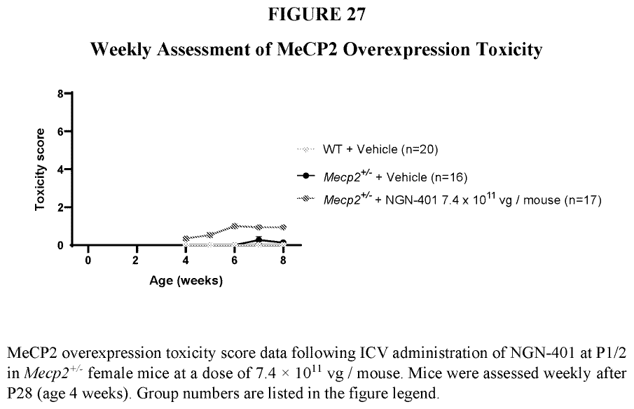

A further in vivo study in female heterozygous Mecp2^{+/-} mice was conducted to assess the tolerability of NGN-401, an MECP2 gene therapy construct in which expression levels are regulated by an EXACT miRNA circuit. In this study, an extremely high dose of 7.4×10¹¹ vg/mouse was used (FIG. 25), the maximum achievable based on the vector titer and injection volume limitations. Previous studies using lower doses of 1.0×10¹¹ and 3.0×10¹¹ vg/mouse did not reveal any evidence of toxic MeCP2 overexpression effects.

The vector was manufactured using a baculovirus production system and administered via intracerebroventricular (ICV) injection at postnatal day 1 or 2 (P1/P2). A total of 8-12 animals per group were used for the 8 week in-life arm of the study. The tolerability of NGN-401 was assessed using an MeCP2 toxicity scoring system (MeCP2 overexpression score) developed at the University of Edinburgh. In this study, animals treated with high dose NGN-401 showed a very mild phenotype, manifesting as abnormal clasping of the hindlimb when lifted by the base of the tail. This represented a score of 1, the lowest score achievable on the overexpression toxicity score. The phenotype stabilized at 6 weeks and then plateaued.

Assessment of vector DNA at 8 weeks of age showed widespread biodistribution across various CNS regions, with highest levels in the cortex and lowest levels in the cerebellum. Western blot analysis showed a similar pattern, with highest MeCP2 levels observed in the cortex. Importantly, even in the highly transduced cortex, transgene derived MeCP2 levels in NGN-401 treated Mecp2^{+/-} mice were maintained within physiological levels and were similar to MeCP2 levels in untreated WT mice.

To identify any histopathological correlates of the mild hindlimb phenotype, eight mice per group were sacrificed at 8 weeks of age and tissues assessed by an expert veterinary neuropathologist. Results showed that NGN-401 did not induce adverse findings in the tissues evaluated.

In conclusion, even at the maximum feasible vector dose that could be administered, NGN-401 treatment in Mecp2^{+/-} mice showed only a very mild in-life hindlimb phenotype which was not associated with any histopathological changes. This contrasts with an unregulated vector, which was previously shown to be highly toxic even at a dose of 1.0×10¹¹ vg/mouse, a dose 7.4 times less than that used for NGN-401 in the current study. This highlights the markedly improved safety window achieved by the EXACT regulated NGN-401 construct.

TABLE 3
Study design

| | | | | | 6-month/ |
|---|---|---|---|---|---|
| | | | | Dose | 2-month |
| Group | Sex | Genotype | Test Article | (vg/mouse) | Necropsy |
| 1 | F | WT | Vehicle | — | 12/8 |
| 2 | F | Mecp2^{+/-} | Vehicle | — | 8/8 |
| 3 | F | Mecp2^{+/-} | NGN-401 | 7.4 × 10¹¹ | 9/8 |

All clinical assessments were carried out by personnel blind to both genotype and treatment. An animal caretaker conducted daily cage side observations for each animal and abnormal findings were flagged. Body weight was recorded once a week from P28 onwards. MeCP2 overexpression toxicity was assessed weekly from P28 onwards using a 6-point scoring system developed at the University of Edinburgh. RTT phenotypes were assessed weekly from P28 onwards using the RTT score, a non-invasive observational scoring system modified from a previous scoring system developed at the University of Edinburgh (Guy et al., 2007). Animals were given a score of 0-5 by a blinded researcher in each of 6 parameters: mobility, gait, hindlimb clasping, tremor, breathing, and general condition. A score of 0 signifies the phenotype of wild-type animals, and a score of 5 represents the most severe phenotype. These scores are then combined to give an aggregate RTT phenotype score.

### In Life Evaluations: Survival

In-life safety was monitored until 8 weeks post-injection (Error! Reference source not found.5). Mecp2^{+/-} mice treated with NGN-401 at 7.4×10¹¹ vg/mouse did not suffer any spontaneous deaths and no animals had to be culled due to reaching a humane endpoint. For vehicle treated Mecp2^{+/-} mice, one animal was culled due to reaching a humane endpoint at approximately 7 weeks of age and was found to have developed hydrocephalus on necropsy. For vehicle treated WT mice, one mouse was found dead at approximately 5 weeks of age and one mouse reached a humane endpoint at approximately 6 weeks of age due to severe hydrocephalus.

### In Life Evaluations: Body Weight

Body weight was recorded weekly for each animal from P28 onwards (Error! Reference source not found.26). NGN-401 treated Mecp2^{+/-} bodyweight was slightly reduced compared to WT animals (at 8 weeks, mean bodyweight of NGN-401 treated Mecp2^{+/-} mice was 15.2 g, compared with 16.8 g for vehicle-treated WT animals). In contrast, vehicle treated Mecp2^{+/-} mice had a slightly higher body weight of 17.4 g. These results suggests that treatment with NGN-401 leads to a very subtle decrease in bodyweight.

### In Life Evaluations: MeCP2 Overexpression Score

Toxicity phenotypes were monitored using a toxicity scoring system that was developed at the University of Edinburgh to classify deleterious effects associated with MeCP2 overexpression. From 5 weeks onwards a very mild phenotype was detectable in the majority of Mecp2^{+/-} mice treated with NGN-401 at a dose of 7.4×10¹¹ vg/mouse (Error! Reference source not found.27). This manifested as an abnormal positioning of the hindlimb when the mouse was suspended from the base of the tail and met the criteria for the lowest score of 1 on the MeCP2 overexpression score. Apart from this specific phenotype mice appeared to be otherwise healthy and normal. This phenotype was not observed in WT or Mecp2^{+/-} treated with vehicle. To identify any histopathological correlates of this mild hindlimb phenotype a cohort of mice were culled at 8 weeks and an extensive set of tissues collected for assessment by a veterinary pathologist.

### In Life Evaluations: RTT Score

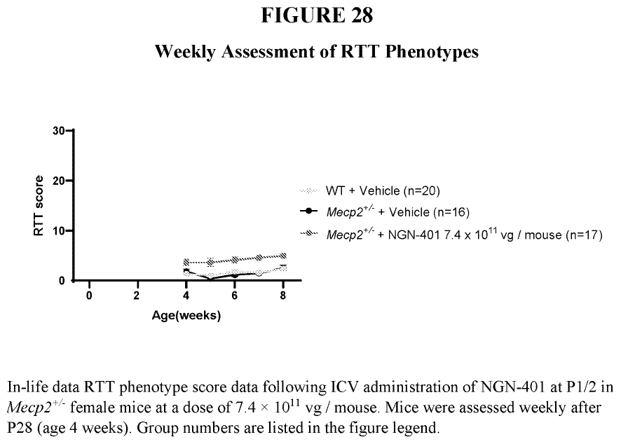

In comparison with the more severe male mouse model, Mecp2^{+/-} female mice display subtle and highly variable phenotypes. For WT and Mecp2^{+/-} mice treated with vehicle, RTT phenotypes were negligible, with a score of around 2.5 out of 30 at 8 weeks of age. At the same age, Mecp2^{+/-} mice treated with NGN-401 displayed a mild phenotype, scoring 5 (FIG. 28). The increase in RTT score in NGN-401-treated mice is due to the presence of the mild hindlimb phenotype. Hindlimb clasping phenotypes are captured by both the RTT score and the MeCP2 overexpression score, as they are usually observed in the Mecp2^{+/-} mice from 5-6 months of age but can also occur because of overexpression toxicity. The phenotype is detectable, but mild.

### Vector Biodistribution

A qPCR assay targeting the WPRE3 component of the NGN-401 vector was used to determine the levels of vector DNA across various regions. For NGN-401 treated mice (Error! Reference source not found. 29) biodistribution was measured at 8 weeks of age in the necropsy arm of the study. Vector genome presence was detected at varying levels in cortex, cerebellum, thoracic spinal cord, and liver in all NGN-401 treated animals, with levels being highest in the cortex (5.4 copies of NGN-401 per diploid genome) and lowest in the cerebellum (0.14 copies of NGN-401 per diploid genome).

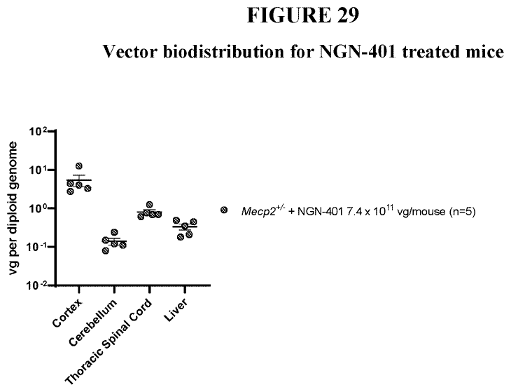

In FIG. 29, vector DNA biodistribution levels are shown at 8-week timepoint in cortex, cerebellum, thoracic spinal cord, and liver after ICV delivery of NGN-401 at a dose of 7.4×10¹¹ vg/mouse. Results are presented as number of vector genome copies per diploid genome determined by a qPCR assay targeting the WPRE3 element of the NGN-401 vector and normalized per diploid genome using an assay targeting the mouse actin gene. Group size numbers are shown in the figure legend. vg=vector genomes.

### MeCP2 Expression: Western Blot

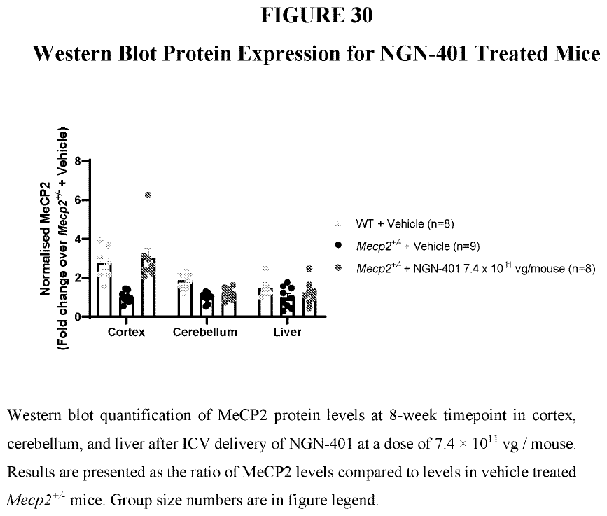

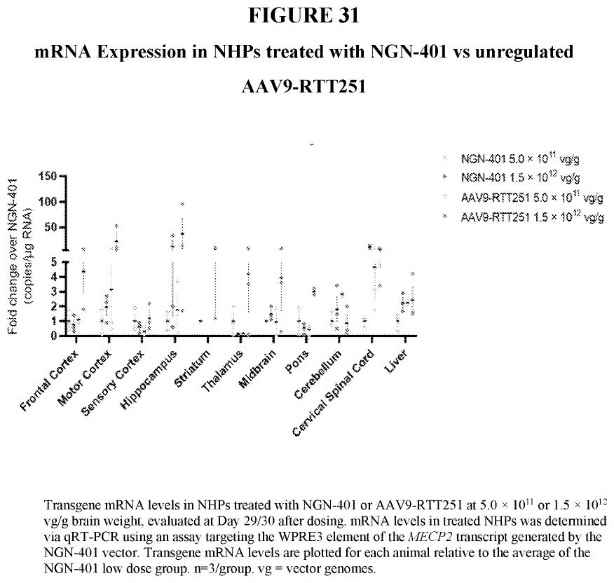

Western blot analysis using an anti-MeCP2 antibody was used to measure protein expression across various regions. The anti-MeCP2 antibody recognizes both mouse and human versions of the protein, therefore measured protein levels were a combination of mouse endogenous MeCP2 protein and vector derived human protein. In the cortex, which had the highest levels of vector biodistribution, mice treated with NGN-401 (FIG. 30) expressed vector derived MeCP2 protein at 200% of levels in vehicle treated Mecp2^{+/-}. In the cerebellum, levels were lower, with vector derived MeCP2 protein at only 16% of levels in vehicle treated Mecp2^{+/-}. Importantly, even at this very high dose in the well transduced cortex, treatment with NGN-401 led to overall MeCP2 protein levels only ˜10% above levels in vehicle treated WT mice. This demonstrates the ability of NGN-401 to maintain protein expression within physiological limits even after treatment with very high doses of NGN-401.

### Histopathology

Histopathological evaluation of tissues collected at the 2-month interim sacrifice was performed by an expert neuropathologist. NGN-401 did not produce adverse findings in the brain (injection site) or any other organs evaluated: Autonomic Ganglia (various, located lateral and/or ventral to vertebrae), Bone with Bone Marrow (vertebrae), Brain (one hemisphere), Forebrain (major regions: cerebral cortex [frontal, parietal, temporal, occipital], striatum, hippocampus, hypothalamus, thalamus), Midbrain, Hind Brain (major regions: cerebellum, pons or occasionally medulla oblongata), Dorsal Root Ganglia (DRG, for cervical and lumbar spinal cord divisions—with spinal nerve roots [mainly for caudal lumbar sections]), Heart (one longitudinal section through ventricles), Intestine, Large—colon cross section (located in the lumbar spinal cord sections), Intestine, Small—jejunum (or occasionally duodenum) cross section, Kidney (one cross section through the hilus), Liver, Lung, Nerves (multiple cross sections of caudal [tail], sciatic, and tibial trunks, and one longitudinal section of sciatic nerve), Ovary, Oviduct, Spinal Cord (cross sections from cervical, thoracic, lumbar [variable], and sacral domains), Skeletal Muscle (gastrocnemius, quadriceps femoris, various tail muscles), Spleen (one longitudinal section), Uterus (one longitudinal section).

### Example 6: NHP Safety (Labcorp); Data for Safety and Regulated Vs Unregulated Expression

Safety and biodistribution studies were initiated in cynomolgus macaques to compare the safety, vector biodistribution and MECP2 expression levels following delivery of the regulated NGN-401 construct (equivalent to RTT254) in comparison to an otherwise equivalent, unregulated MECP2 construct (AAV9-RTT251) in a large animal model.

NGN-401 or AAV9-RTT251 was administered by ICV injection at two doses separated by a half-log (Table 4) as determined using a qualified ddPCR titer assay targeting the human MECP2 transgene. The NGN-401 and AAV9-RTT251 vectors used in this study were produced using a baculovirus system at Virovek. Doses were selected to be within the NGN-401 dose range shown to be efficacious in the Mecp2^{-/y} efficacy studies. The study was designed to reveal any potential NGN-401-related toxicities, including potential toxicity due to overexpression of MeCP2, by evaluating a higher dose than previously tested in NHPs and including the comparison to AAV9-RTT251, the unregulated vector described above. The AAV9-RTT251 treatment groups were sacrificed one month following vector dosing to avoid potential for severe toxicity, given the known toxicity observed in previous mouse studies. Animals received daily oral prednisolone (1 mg/kg) beginning two weeks prior to vector dosing and continued throughout the duration of the study.

TABLE 4
Design of NGN-2021-012 Safety and Biodistribution Study in NHPs

| | | | Dosage Level (based on GOI ddPCR) | | | | |
|---|---|---|---|---|---|---|---|
| | | Test | Concentration | Volume | Total Dose | Dose | Sacrifice |
| Group | N | Article | (vg/mL) | (mL) | (vg)^{a} | (vg/g brain)^{b} | Timepoint |
| 1 | 2M/1F | NGN-401 | 2.85 × 10¹³ | 1.3 | 3.7 × 10¹³ | 5.0 × 10¹¹ | 30 days |
| 2 | 1M/2F | NGN-401 | 8.50 × 10¹³ | 1.3 | 1.1 × 10¹⁴ | 1.5 × 10¹² | 30 days |
| 3 | 1M/2F | NGN-401 | 8.50 × 10¹³ | 1.3 | 1.1 × 10¹⁴ | 1.5 × 10¹² | 92 days |
| 4 | 2M/1F | AAV9- | 2.85 × 10¹³ | 1.3 | 3.7 × 10¹³ | 5.0 × 10¹¹ | 30 days |
| | | RTT251 | | | | | |
| 5 | 2M/1F | AAV9- | 1.10 × 10¹⁴ | 1.0 | 1.1 × 10¹⁴ | 1.5 × 10¹² | 30 days |
| | | RTT251 | | | | | |

^{a}Titer used at dosing did not change following evaluation using qualified MECP2 ddPCR assay
^{b}Cynomolgus macaque brain weight = 74 g

The study included assessment of clinical pathology and neurobehavioral observation, as well as anatomic pathology. All animals survived to the scheduled sacrifice, and no test article-related clinical or neurobehavioral observations, changes in body weight or food consumption, or macroscopic findings were noted with either NGN-401 or AAV9-RTT251.

Clinical pathology effects with both NGN-401 and AAV9-RTT251 consisted of minimally or mildly increased platelet, white blood cell, absolute neutrophil and absolute monocyte counts, fibrinogen concentration and alanine aminotransferase (ALT) activity. The increased platelet and leukocyte counts and fibrinogen concentration were suggestive of an inflammatory response. Of the animals with collections on Day 92, values generally decreased towards baseline by this timepoint. The transiently increased alanine aminotransferase activity has been reported in AAV9 studies and lacked correlative liver weight changes or microscopic findings in the liver. Asymptomatic increase in ALT is an expected effect of AAV gene therapy. Transiently increased ALT activity has been reported in AAV studies involving intravenous (IV) and intrathecal (IT) routes of administration with greater hepatic exposure which were successfully managed with steroid treatment.

Test article-related microscopic observations with both vectors were noted in the cervical, thoracic, and/or lumbar dorsal root ganglia (mononuclear cell infiltrate, neuronal cell loss, and/or axonal degeneration) and/or the spinal cord (axonal degeneration in the nerve roots and/or white matter). These findings had no dose relationship for incidence or severity, were similar at interim and terminal sacrifices and were considered not adverse. This is aligned with the current understanding of the safety profile for AAV gene therapy. Histopathological findings in dorsal root ganglia (DRG) are an expected effect of AAV gene therapy as clinically asymptomatic findings have been reported in AAV studies with NHPs. Currently available data suggests that DRG pathology is almost universal after AAV gene therapy in nonclinical studies using NHPs and has not resulted in clinical signs after treatment at therapeutic transgene doses.

Nerve conduction measurement was performed in upper (radial and median) and lower (sural, peroneal, and saphenous) sensory nerves at Week 3/4 and Week 13 of the study. A reduction in nerve conduction velocity (NCV) ≥3 m/s from baseline was considered NCV slowing. The summary of the incidence of sensory NCV slowing in each treatment group is provided in Table 5.

TABLE 5
Number of NHPs Exhibiting Sensory NCV Slowing in NGN-2021-012

| Group | Radial | Median | Sural | Peroneal | Saphenous |
|---|---|---|---|---|---|
| NGN-401 | | | | | |
| 5.0 × 10¹¹ vg/g | | | | | |
| (n = 3) | | | | | |
| NGN-401 | | | 1 of 6^{a} | 1 of 6^{a} | |
| 1.5 × 10¹² vg/g | | | 1 of 3^{b} | | |
| (n = 6) | | | | | |
| AAV9- | | | 3 of 3^{a} | 1 of 3^{a} | |
| RTT251 | | | (no response | | |
| 5.0 × 10¹¹ vg/g | | | in 2) | | |
| (n = 3) | | | | | |
| AAV9- | | | 2 of 3^{a} | | 1 of 3^{a} |
| RTT251 | | | | | |
| 1.5 × 10¹² vg/g | | | | | |
| (n = 3) | | | | | |

^{a}Finding at Week 3/4
^{b}Finding at Week 13

No sensory functional loss was noted for the radial or median nerves at any time point. At Week 3/4, two animals that received the low dose of AAV9-RTT251 exhibited no response in the sural nerve, and one animal exhibited NCV slowing. This was considered severe and occurred with increased incidence when compared to the other groups. All other findings indicated low incidence of minor or moderate impact on the respective nerve function, and respective nerves are expected to remain within physiologically functional parameters. Of note, a single animal in the low dose AAV9-RTT251 group was noted with a minor F-M wave latency difference latency prolongation during Weeks 3/4, potentially indicating a slowing of motor conduction in the proximal portion of the tibial nerve.

Overall, the sural NCV data highlight a difference in the safety profile of NGN-401 when compared to an otherwise equivalent MECP2 vector that does not contain EXACT self-regulation technology, as two of nine NGN-401 treated animals exhibited a sural NCV slowing, while five of six AAV9-RTT251 treated animals exhibited sural NCV slowing.

One-month following ICV administration of NGN-401 and AAV9-RTT251, select tissues (brain, liver, spinal cord) were collected for evaluation of mRNA expression. Transgene expression was evaluated using a qRT-PCR assay targeting the WPRE3 element in the 3′ UTR of the MECP2 mRNA. The impact of the EXACT technology in NGN-401 is apparent when the mRNA levels produced by AAV9-RTT251 are directly compared to those produced by NGN-401. The transgene mRNA levels (copies per g RNA) for each dose group were normalized to the NGN-401 low dose group in each region evaluated (Error! Reference source not found.). In a majority of regions, AAV9-RTT251 produced mRNA levels that were several fold higher than the levels in the corresponding group that received an equivalent dose of NGN-401, and also exhibited more variability between animals. This data provides evidence that EXACT technology is able to regulate expression levels in CNS tissues in a large animal model.

These data illustrate that several optimized therapeutic polynucleotide cassettes are good candidates for effective gene therapy for Rett syndrome and avoid the safety/toxicity concerns.

### Clinical Design

A clinical dose has been designed and proposed for a clinical trial wherein the subject is dosed with 1.0×10¹⁵ vg, delivered via a 10 mL ICV injection at 1.0×10¹⁴ vg/mL.

Based on an average female brain weight, 1E15 vg scales to 8.3×10¹¹ vg/g brain, but actual dose per gram of brain weight could be higher in females with Rett syndrome due to smaller brain size. For some patients this will work out to an effective dose of about 8.3×10¹¹ vg/g brain. It is expected that the dosed subjects will be substantially free of MECP2 overexpression toxicity, in line with the GLP and NHP safety and tox studies provided herein above. Additionally, endpoints will be mapped to preclinical data according to the improvements in various domains in animal models as shown below.

### Mapping of Endpoints to Preclinical Data

| Improvement in | |
|---|---|
| various domains in | |
| Rett animal models | Measures in humans that can map to animal data |
| Apnea/Breathing | Apnea/Breathing - CGI breathing, |
| | Emerald/RSBQ |
| Motor | Motor Improvement - CGI motor, |
| Improvement/Gait | aspects, RSBQ, Rett Syndrome Gross Motor |
| | Scale |
| Autonomic | Breathing items from CGI and RSBQ, Heart |
| dysfunction | rate variability, GI symptoms, Emerald findings, |
| | constipation, HR and BP variability |

SEQUENCE LISTING AND FEATURES

| SEQ ID NO | Construct |
|---|---|
| SEQ ID NO: 1 | CMV/CBA |
| SEQ ID NO: 2 | MINIX_intron |
| SEQ ID NO: 3 | EF1a_intron |
| SEQ ID NO: 4 | EXACT1 (ffluc1) |
| SEQ ID NO: 5 | EXACT2 (ran1g) |
| SEQ ID NO: 6 | EXACT3 (ran2g) |
| SEQ ID NO: 7 | hMECP2 |
| SEQ ID NO: 8 | 3xEXACT1_binding (ffluc1) |
| SEQ ID NO: 9 | 3xEXACT2_binding (ran1g) |
| SEQ ID NO: 10 | 3xEXACT3_binding (ran2g) |
| SEQ ID NO: 11 | WPRE3 |
| SEQ ID NO: 12 | Sv40polyA |
| SEQ ID NO: 13 | Preferred Kozak sequence |
| SEQ ID NO: 14 | RTT252_CBE-ffluc1-hsaMECP2- |
| | 3xbinding-SV40pA |
| SEQ ID NO: 15 | RTT253_CBE-ffluc1-hsaMECP2- |
| | 3xbinding-WPRE3-SV40pA |
| SEQ ID NO: 16 | RTT254_CBM-ffluc1-hsaMECP2- |
| | 3xbinding-WPRE3-SV40pA |
| SEQ ID NO: 17 | RTT269_CBE-ffluc1-hsaMECP2- |
| | 3xbinding_mut3-WPRE3-SV40pA |
| SEQ ID NO: 18 | RTT270_CBE-ffluc1-hsaMECP2- |
| | 3xbinding_mut6-WPRE3-SV40pA |
| SEQ ID NO: 19 | RTT271_CBE-ran1g-hsaMECP2- |
| | 3xbinding-WPRE3-SV40pA |
| SEQ ID NO: 20 | RTT272_CBE-ran2g-hsaMECP2- |
| | 3xbinding-WPRE3-SV40pA |
| SEQ ID NO: 21 | CBM promoter |
| SEQ ID NO: 22 | CBE promoter |
| SEQ ID NO: 23 | Methyl-CpG Binding Domain (MBD) of |
| | MeCP2 |
| SEQ ID NO: 24 | NCOR/SMRT Interaction Domain (NID) of |
| | MeCP2 |
| SEQ ID NO: 25 | RTT254/NGN-401 |

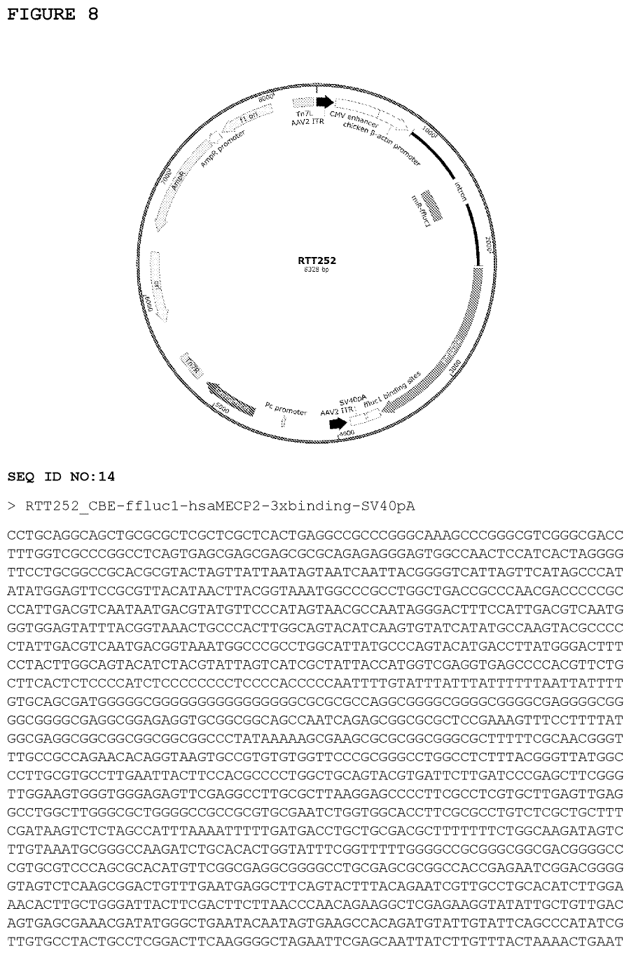

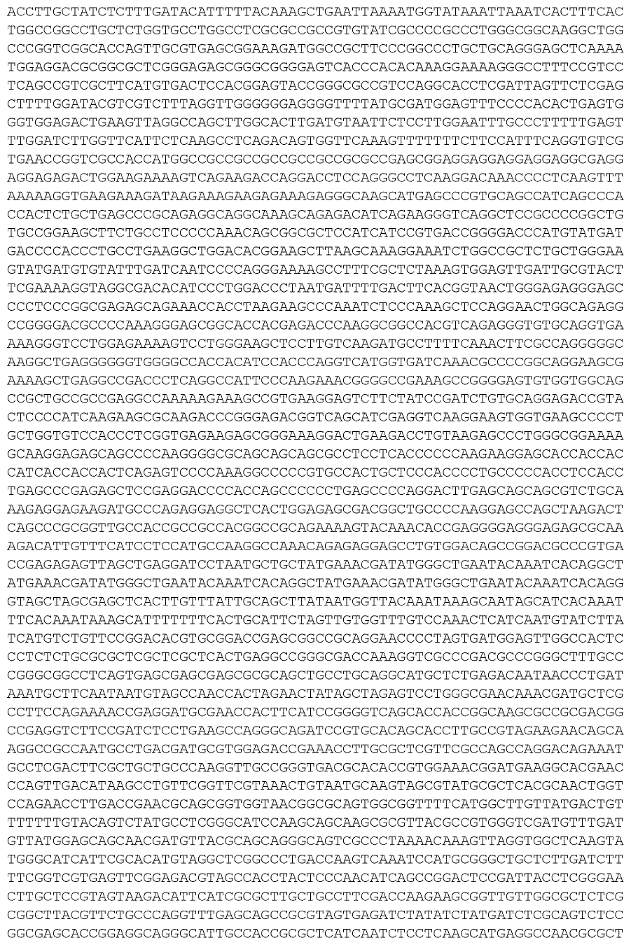

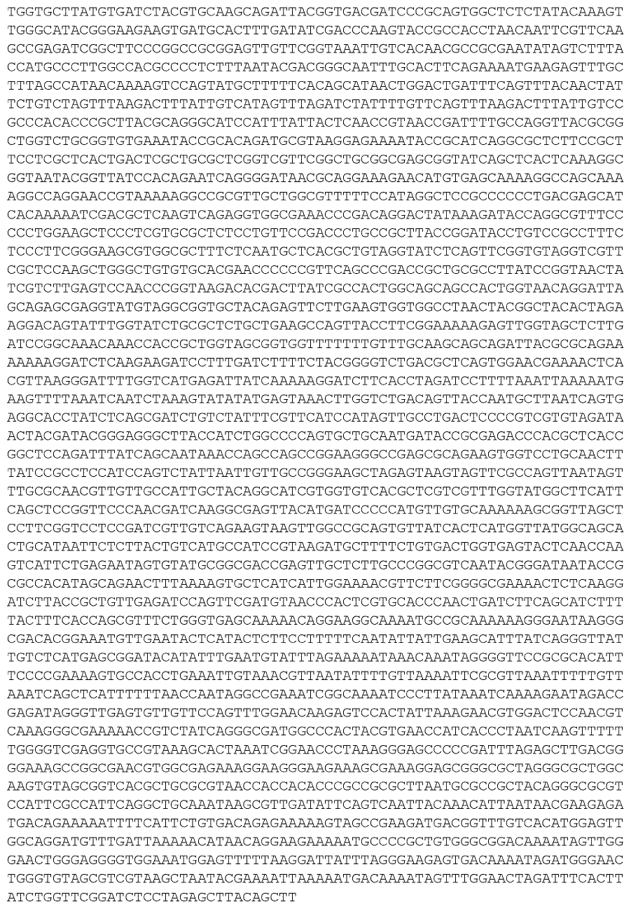

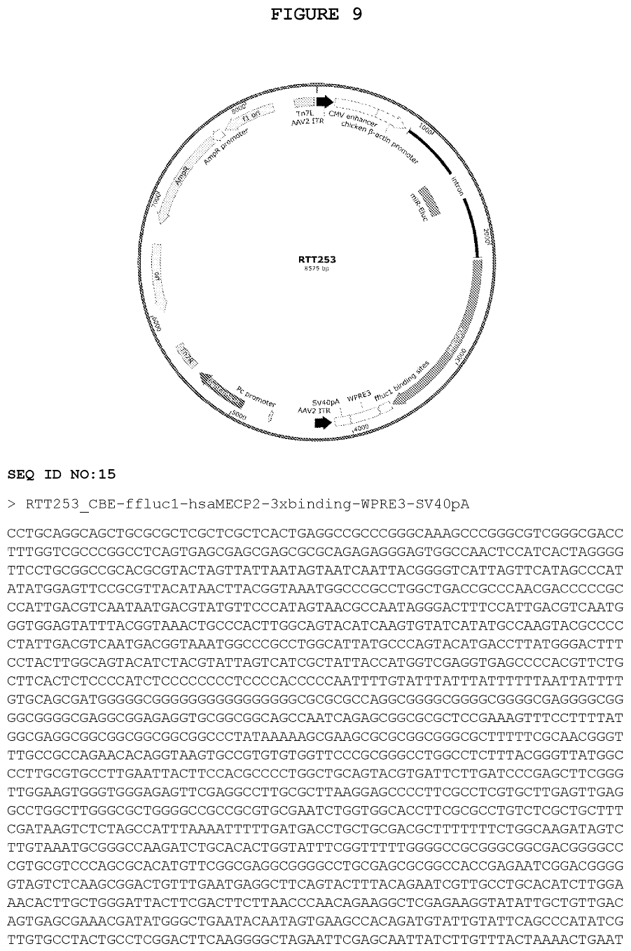

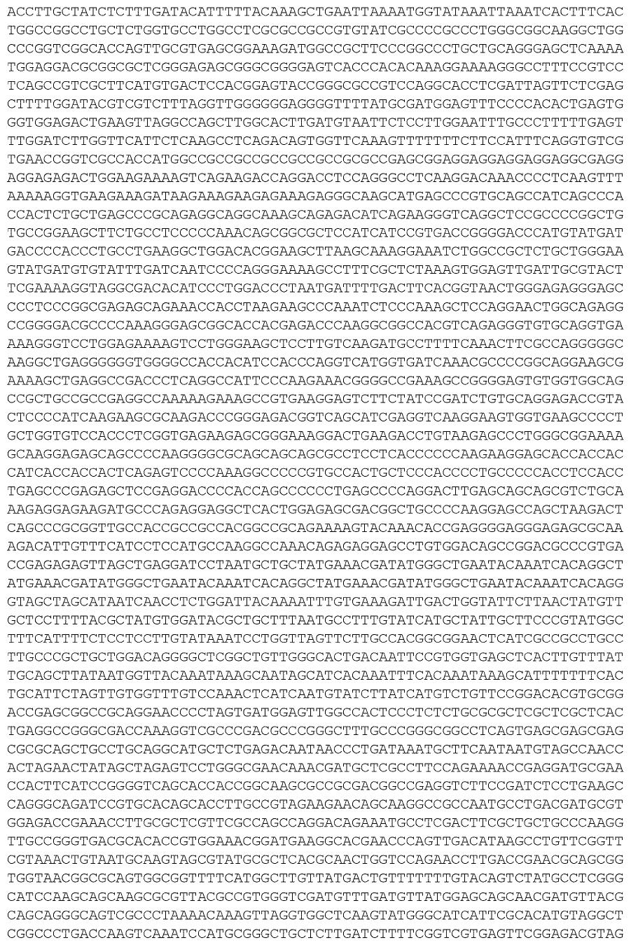

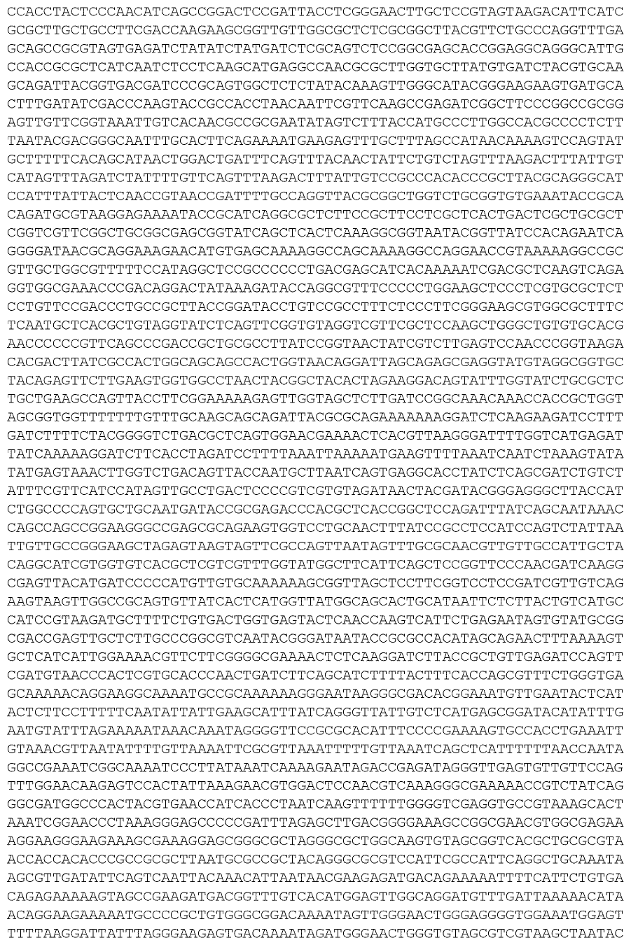

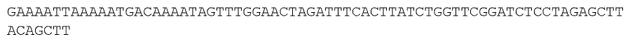

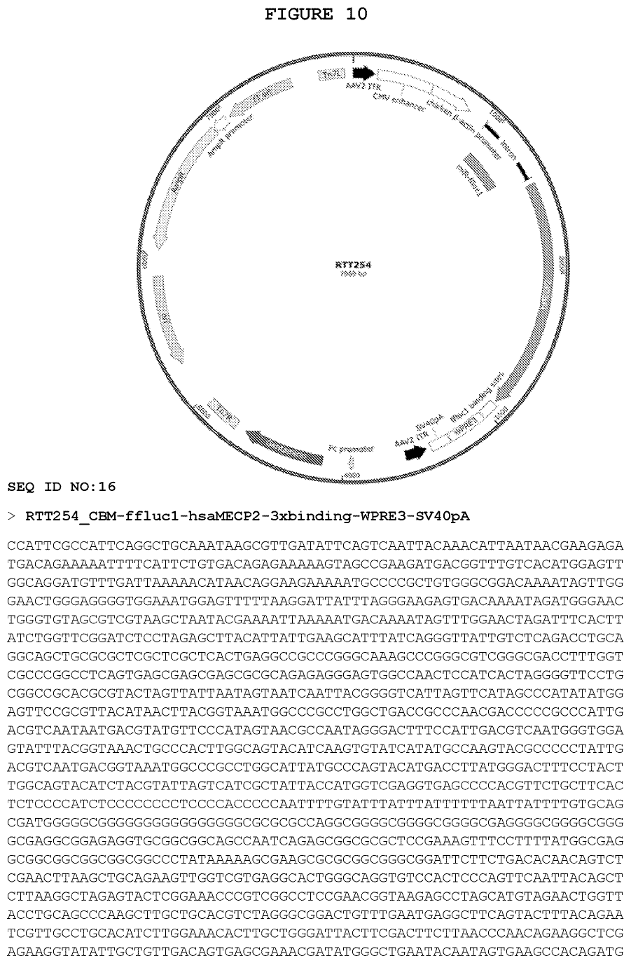

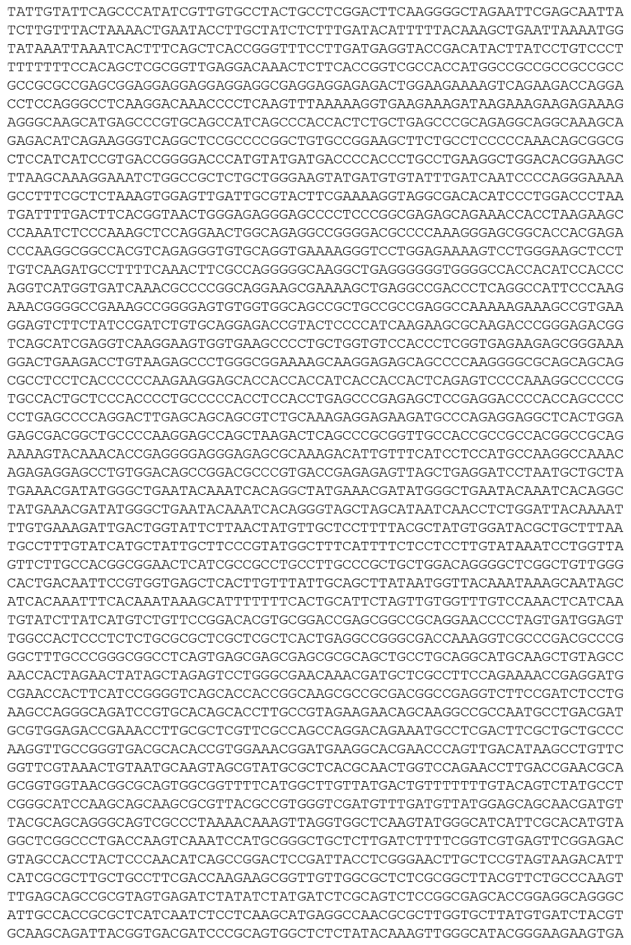

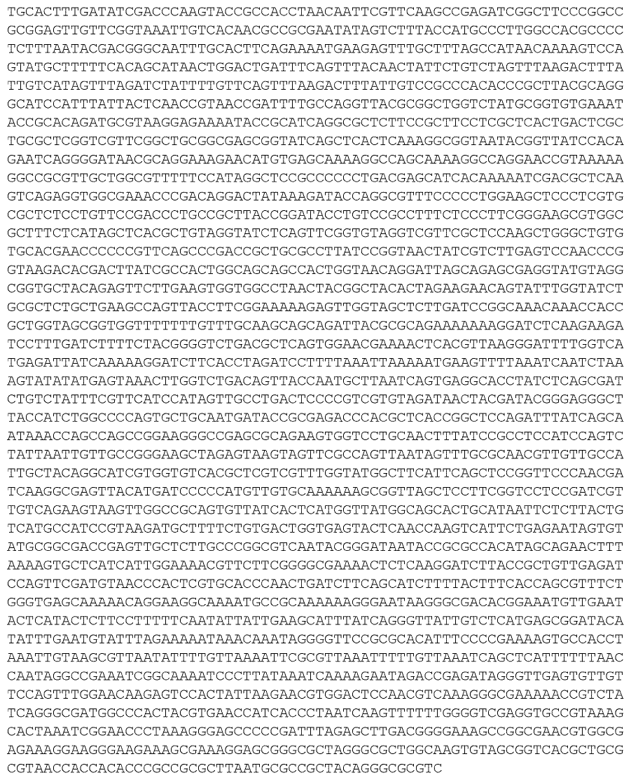

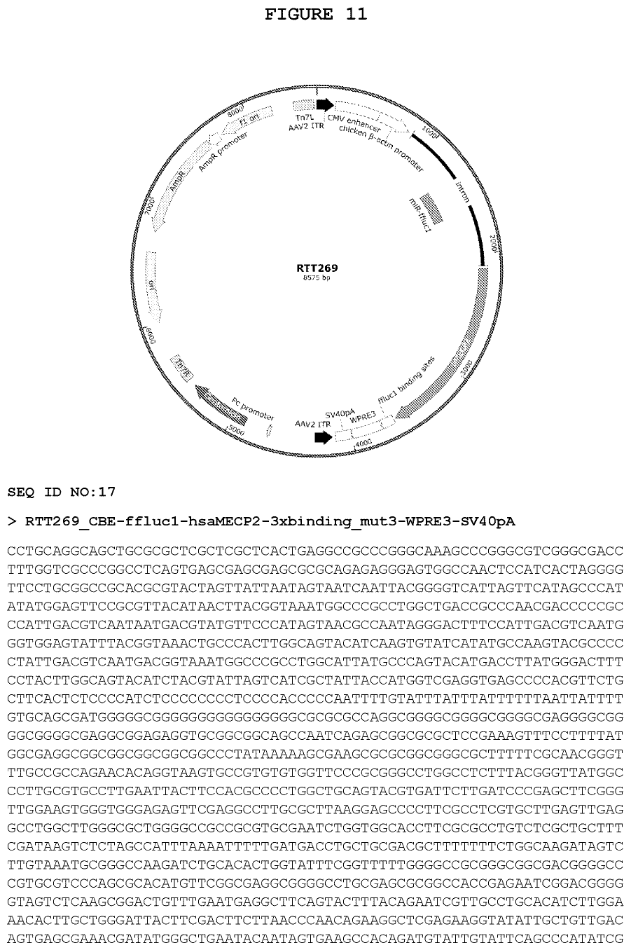

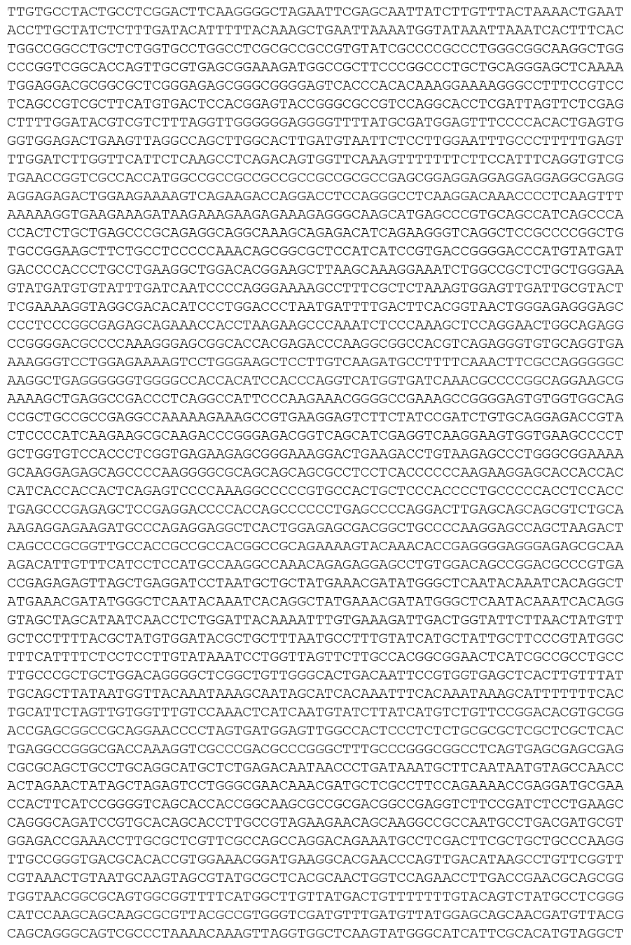

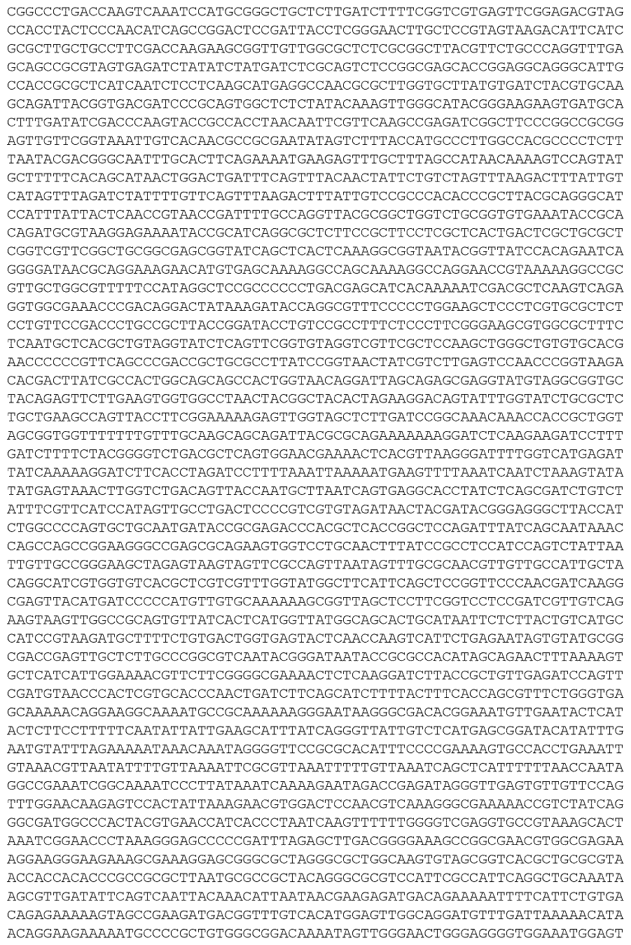

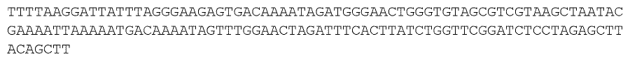

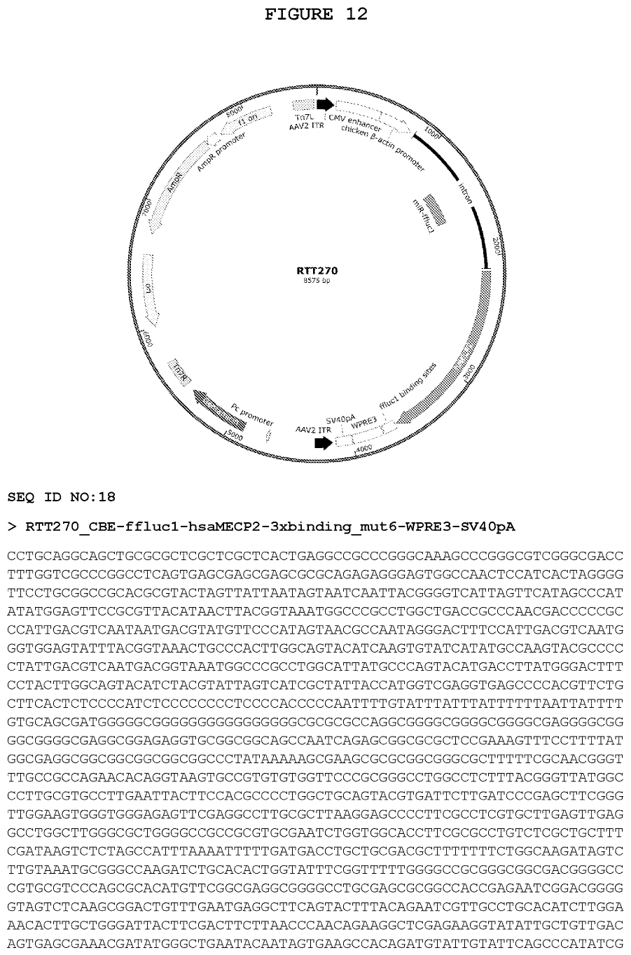

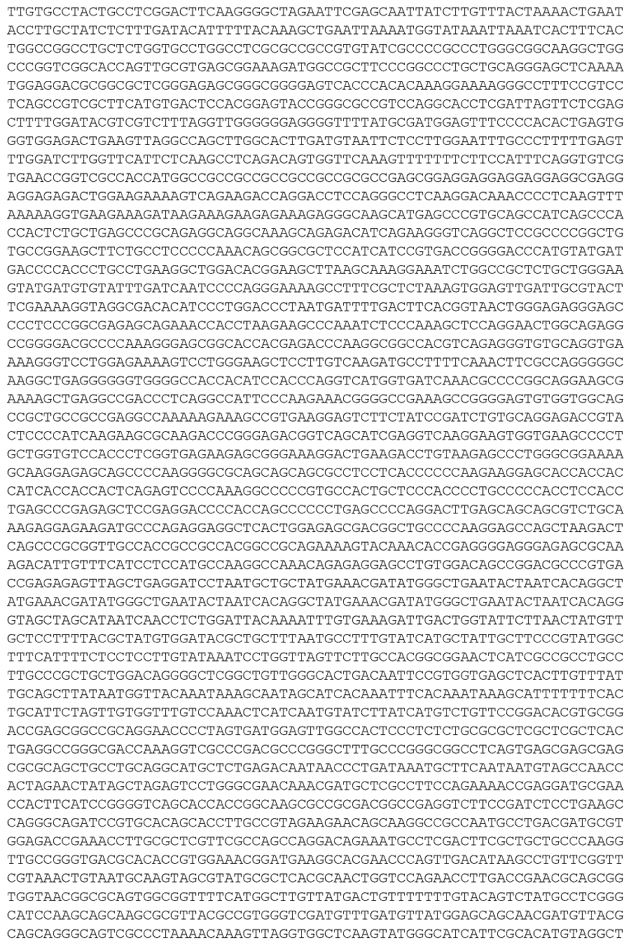

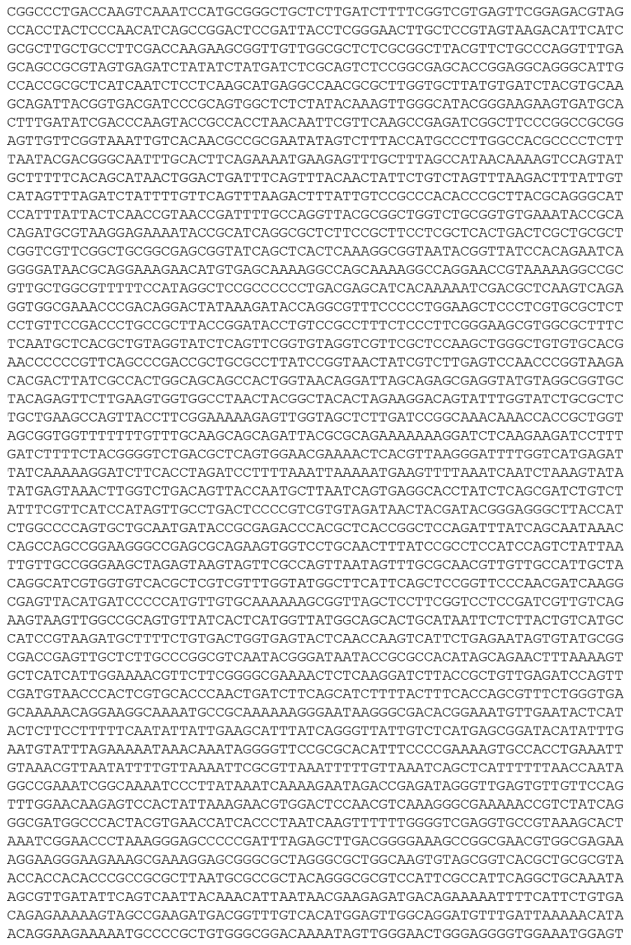

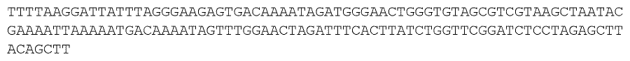

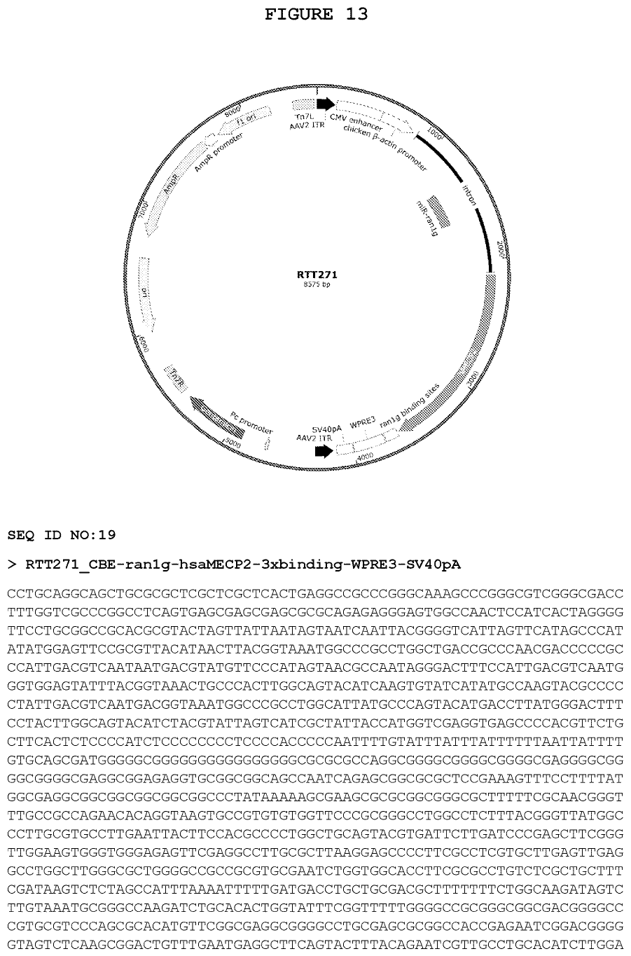

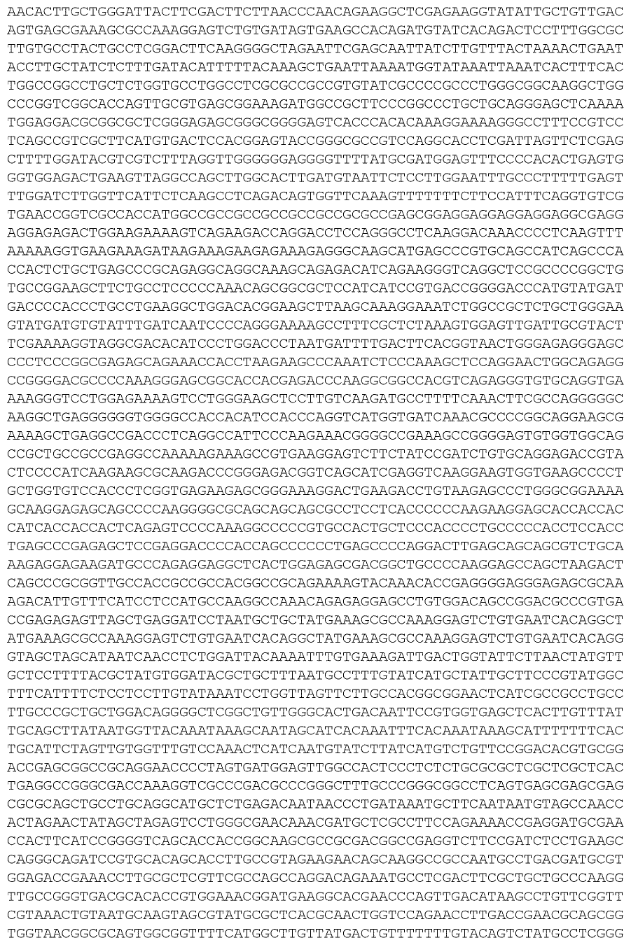

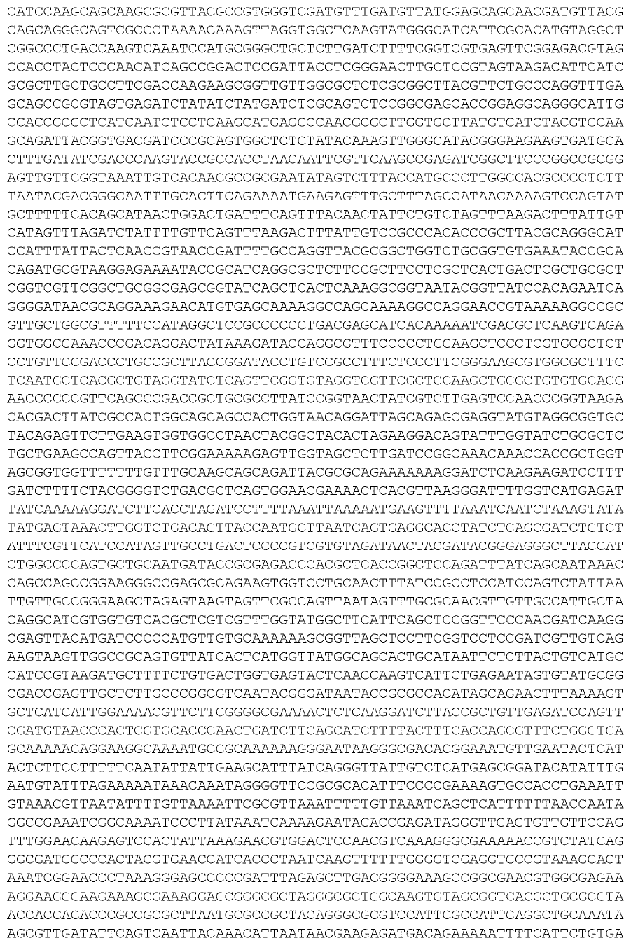

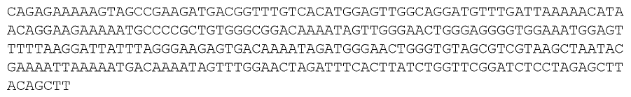

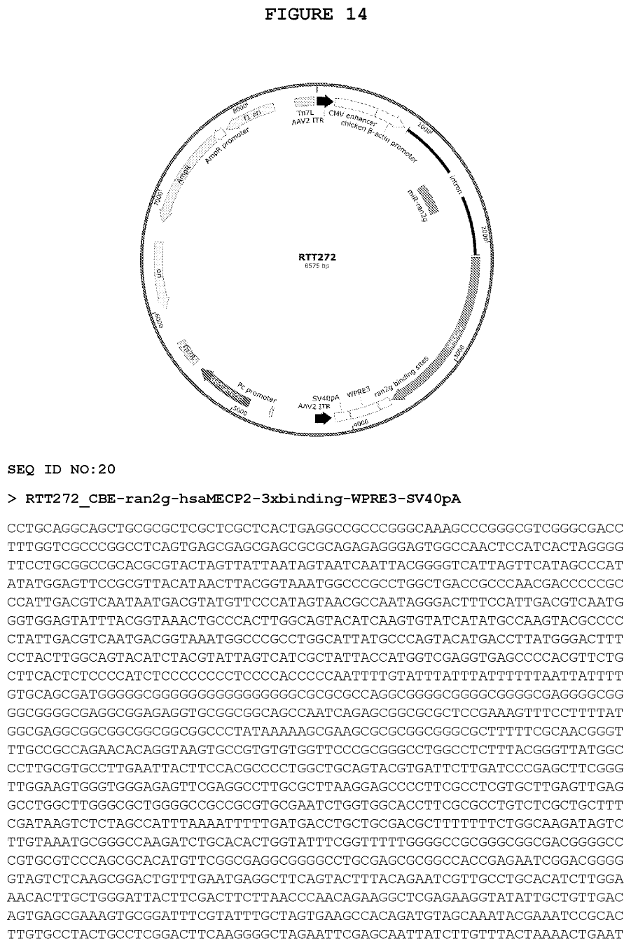

SEQ ID NO: 1
PROMOTERS
>CMV/CBA
CTAGTTATTAATAGTAATCAATTACGGGGTCATTAGTTCATAGCCCATATATGGAGTTCCGCGTTACATAACTT
ACGGTAAATGGCCCGCCTGGCTGACCGCCCAACGACCCCCGCCCATTGACGTCAATAATGACGTATGTTCCCA
TAGTAACGCCAATAGGGACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAATGGCCCGCCTGGCATTAT
GCCCAGTACATGACCTTATGGGACTTTCCTACTTGGCAGTACATCTACGTATTAGTCATCGCTATTACCATGGT
CGAGGTGAGCCCCACGTTCTGCTTCACTCTCCCCATCTCCCCCCCCTCCCCACCCCCAATTTTGTATTTATTTATT
TTTTAATTATTTTGTGCAGCGATGGGGGGGGGGGGGGGGGGGGGGGGCGCGCCAGGCGGGGGGGGGGGG
GGCGAGGGGCGGGGCGGGGCGAGGCGGAGAGGTGCGGCGGCAGCCAATCAGAGCGGCGCGCTCCGAAAG
TTTCCTTTTATGGCGAGGCGGCGGCGGCGGCGGCCCTATAAAAAGCGAAGCGCGCGGCGGGCG
SEQ ID NO: 2
INTRONS
>MINIX_intron
GTAAGAGCCTAGCATGTAGAACTGGTTACCTGCAGCCCAAGCTTGCTGCACGTCTAGGGCCTCACCGGGTTTC
CTTGATGAGGTACCGACATACTTATCCTGTCCCTTTTTTTTCCACAG
SEQ ID NO: 3
>EF1a_intron
GTAAGTGCCGTGTGTGGTTCCCGCGGGCCTGGCCTCTTTACGGGTTATGGCCCTTGCGTGCCTTGAATTACTTC
CACGCCCCTGGCTGCAGTACGTGATTCTTGATCCCGAGCTTCGGGTTGGAAGTGGGTGGGAGAGTTCGAGGC
CTTGCGCTTAAGGAGCCCCTTCGCCTCGTGCTTGAGTTGAGGCCTGGCTTGGGCGCTGGGGCCGCCGCGTGC
GAATCTGGTGGCACCTTCGCGCCTGTCTCGCTGCTTTCGATAAGTCTCTAGCCATTTAAAATTTTTGATGACCTG
CTGCGACGCTTTTTTTCTGGCAAGATAGTCTTGTAAATGCGGGCCAAGATCTGCACACTGGTATTTCGGTTTTT
GGGGCCGCGGGCGGCGACGGGGCCCGTGCGTCCCAGCGCACATGTTCGGCGAGGCGGGGCCTGCGAGCGC
GGCCACCGAGAATCGGACGGGGGTAGTCTCAAGCTGGCCGGCCTGCTCTGGTGCCTGGCCTCGCGCCGCCGT
GTATCGCCCCGCCCTGGGCGGCAAGGCTGGCCCGGTCGGCACCAGTTGCGTGAGCGGAAAGATGGCCGCTTC
CCGGCCCTGCTGCAGGGAGCTCAAAATGGAGGACGCGGCGCTCGGGAGAGCGGGGGGGGAGTCACCCACA
CAAAGGAAAAGGGCCTTTCCGTCCTCAGCCGTCGCTTCATGTGACTCCACGGAGTACCGGGCGCCGTCCAGGC
ACCTCGATTAGTTCTCGAGCTTTTGGATACGTCGTCTTTAGGTTGGGGGGAGGGGTTTTATGCGATGGAGTTT
CCCCACACTGAGTGGGTGGAGACTGAAGTTAGGCCAGCTTGGCACTTGATGTAATTCTCCTTGGAATTTGCCC
TTTTTGAGTTTGGATCTTGGTTCATTCTCAAGCCTCAGACAGTGGTTCAAAGTTTTTTTCTTCCATTTCAG
MICRORNA EXPRESSION
SEQ ID NO: 4
>EXACT1 (ffluc1)
TGTTTGAATGAGGCTTCAGTACTTTACAGAATCGTTGCCTGCACATCTTGGAAACACTTGCTGGGATTACTTCG
ACTTCTTAACCCAACAGAAGGCTCGAGAAGGTATATTGCTGTTGACAGTGAGCGAAACGATATGGGCTGAAT
ACAATAGTGAAGCCACAGATGTATTGTATTCAGCCCATATCGTTGTGCCTACTGCCTCGGACTTCAAGGGGCT
AGAATTCGAGCAATTATCTTGTTTACTAAAACTGAATACCTTGCTATCTCTTTGATACATTTTTACAAAGCTGAA
TTAAAATGGTATAAATTAAATCACTTT
SEQ ID NO: 5
>EXACT2 (ran1g)
TGTTTGAATGAGGCTTCAGTACTTTACAGAATCGTTGCCTGCACATCTTGGAAACACTTGCTGGGATTACTTCG
ACTTCTTAACCCAACAGAAGGCTCGAGAAGGTATATTGCTGTTGACAGTGAGCGAAAGCGCCAAAGGAGTCT
GTGATAGTGAAGCCACAGATGTATCACAGACTCCTTTGGCGCTTGTGCCTACTGCCTCGGACTTCAAGGGGCT
AGAATTCGAGCAATTATCTTGTTTACTAAAACTGAATACCTTGCTATCTCTTTGATACATTTTTACAAAGCTGAA
TTAAAATGGTATAAATTAAATCACTTT
SEQ ID NO: 6
>EXACT3 (ran2g)
TGTTTGAATGAGGCTTCAGTACTTTACAGAATCGTTGCCTGCACATCTTGGAAACACTTGCTGGGATTACTTCG
ACTTCTTAACCCAACAGAAGGCTCGAGAAGGTATATTGCTGTTGACAGTGAGCGAAAGTGCGGATTTCGTATT
TGCTAGTGAAGCCACAGATGTAGCAAATACGAAATCCGCACTTGTGCCTACTGCCTCGGACTTCAAGGGGCTA
GAATTCGAGCAATTATCTTGTTTACTAAAACTGAATACCTTGCTATCTCTTTGATACATTTTTACAAAGCTGAAT
TAAAATGGTATAAATTAAATCACTTT
TRANSGENE
SEQ ID NO: 7
>hMECP2
ATGGCCGCCGCCGCCGCCGCCGCGCCGAGCGGAGGAGGAGGAGGAGGCGAGGAGGAGAGACTGGAAGAA
AAGTCAGAAGACCAGGACCTCCAGGGCCTCAAGGACAAACCCCTCAAGTTTAAAAAGGTGAAGAAAGATAAG
AAAGAAGAGAAAGAGGGCAAGCATGAGCCCGTGCAGCCATCAGCCCACCACTCTGCTGAGCCCGCAGAGGC
AGGCAAAGCAGAGACATCAGAAGGGTCAGGCTCCGCCCCGGCTGTGCCGGAAGCTTCTGCCTCCCCCAAACA
GCGGCGCTCCATCATCCGTGACCGGGGACCCATGTATGATGACCCCACCCTGCCTGAAGGCTGGACACGGAA
GCTTAAGCAAAGGAAATCTGGCCGCTCTGCTGGGAAGTATGATGTGTATTTGATCAATCCCCAGGGAAAAGC
CTTTCGCTCTAAAGTGGAGTTGATTGCGTACTTCGAAAAGGTAGGCGACACATCCCTGGACCCTAATGATTTTG
ACTTCACGGTAACTGGGAGAGGGAGCCCCTCCCGGCGAGAGCAGAAACCACCTAAGAAGCCCAAATCTCCCA
AAGCTCCAGGAACTGGCAGAGGCCGGGGACGCCCCAAAGGGAGCGGCACCACGAGACCCAAGGCGGCCAC
GTCAGAGGGTGTGCAGGTGAAAAGGGTCCTGGAGAAAAGTCCTGGGAAGCTCCTTGTCAAGATGCCTTTTCA
AACTTCGCCAGGGGGCAAGGCTGAGGGGGGTGGGGCCACCACATCCACCCAGGTCATGGTGATCAAACGCC
CCGGCAGGAAGCGAAAAGCTGAGGCCGACCCTCAGGCCATTCCCAAGAAACGGGGCCGAAAGCCGGGGAGT
GTGGTGGCAGCCGCTGCCGCCGAGGCCAAAAAGAAAGCCGTGAAGGAGTCTTCTATCCGATCTGTGCAGGA
GACCGTACTCCCCATCAAGAAGCGCAAGACCCGGGAGACGGTCAGCATCGAGGTCAAGGAAGTGGTGAAGC
CCCTGCTGGTGTCCACCCTCGGTGAGAAGAGCGGGAAAGGACTGAAGACCTGTAAGAGCCCTGGGCGGAAA
AGCAAGGAGAGCAGCCCCAAGGGGCGCAGCAGCAGCGCCTCCTCACCCCCCAAGAAGGAGCACCACCACCA
TCACCACCACTCAGAGTCCCCAAAGGCCCCCGTGCCACTGCTCCCACCCCTGCCCCCACCTCCACCTGAGCCCG
AGAGCTCCGAGGACCCCACCAGCCCCCCTGAGCCCCAGGACTTGAGCAGCAGCGTCTGCAAAGAGGAGAAG
ATGCCCAGAGGAGGCTCACTGGAGAGCGACGGCTGCCCCAAGGAGCCAGCTAAGACTCAGCCCGCGGTTGC
CACCGCCGCCACGGCCGCAGAAAAGTACAAACACCGAGGGGAGGGAGAGCGCAAAGACATTGTTTCATCCTC
CATGCCAAGGCCAAACAGAGAGGAGCCTGTGGACAGCCGGACGCCCGTGACCGAGAGAGTTAGCTGA
BINDING SITES
SEQ ID NO: 8
>3xEXACT1_binding (ffluc1)
GCTATGAAACGATATGGGCTGAATACAAATCACAGGCTATGAAACGATATGGGCTGAATACAAATCACAGGC
TATGAAACGATATGGGCTGAATACAAATCACAG
SEQ ID NO: 9
>3xEXACT2_binding (ran1g)
GCTATGAAAGCGCCAAAGGAGTCTGTGAATCACAGGCTATGAAAGCGCCAAAGGAGTCTGTGAATCACAGGC
TATGAAAGCGCCAAAGGAGTCTGTGAATCACAG
SEQ ID NO: 10
>3xEXACT3_binding (ran2g)
GCTATGAAAGTGCGGATTTCGTATTTGCATCACAGGCTATGAAAGTGCGGATTTCGTATTTGCATCACAGGCT
ATGAAAGTGCGGATTTCGTATTTGCATCACAG
STABILITY ELEMENT
SEQ ID NO: 11
>WPRE3
ATAATCAACCTCTGGATTACAAAATTTGTGAAAGATTGACTGGTATTCTTAACTATGTTGCTCCTTTTACGCTAT
GTGGATACGCTGCTTTAATGCCTTTGTATCATGCTATTGCTTCCCGTATGGCTTTCATTTTCTCCTCCTTGTATAA
ATCCTGGTTAGTTCTTGCCACGGCGGAACTCATCGCCGCCTGCCTTGCCCGCTGCTGGACAGGGGCTCGGCTG
TTGGGCACTGACAATTCCGTGGT
POLYADENYLATION SIGNAL
SEQ ID NO: 12
>SV40pA
ACTTGTTTATTGCAGCTTATAATGGTTACAAATAAAGCAATAGCATCACAAATTTCACAAATAAAGCATTTTTTT
CACTGCATTCTAGTTGTGGTTTGTCCAAACTCATCAATGTATCTTATCATGTCTGT
SEQ ID NO: 13
Kozak sequence
GCCACCATGG
SEQ ID 14-20 are in FIGS. 8-14
SEQ ID NO: 21
>CBM Promoter
CTAGTTATTAATAGTAATCAATTACGGGGTCATTAGTTCATAGCCCATATATGGAGTTCCGCGTTACA
TAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAA
CTGCCCACTTGGCAGTACATCAAGTGTATCATATGCCAAGTACGCCCCCTATTGACGTCAATGACGGT
AAATGGCCCGCCTGGCATTATGCCCAGTACATGACCTTATGGGACTTTCCTACTTGGCAGTACATCTA
CGTATTAGTCATCGCTATTACCATGGTCGAGGTGAGCCCCACGTTCTGCTTCACTCTCCCCATCTCCC
CCCCCTCCCCACCCCCAATTTTGTATTTATTTATTTTTTAATTATTTTGTGCAGCGATGGGGGCGGGG
GGGGGGGGGGGGCGCGCGCCAGGCGGGGCGGGGCGGGGCGAGGGGCGGGGCGGGGCGAGGCGGAGAGG
TGCGGCGGCAGCCAATCAGAGCGGCGCGCTCCGAAAGTTTCCTTTTATGGCGAGGCGGCGGCGGCGGC
GGCCCTATAAAAAGCGAAGCGCGCGGCGGGCGGATTCTTCTGACACAACAGTCTCGAACTTAAGCTGC
AGAAGTTGGTCGTGAGGCACTGGGCAGGTGTCCACTCCCAGTTCAATTACAGCTCTTAAGGCTAGAGT
ACTCGGAAACCCGTCGGCCTCCGAACGGTAAGAGCCTAGCATGTAGAACTGGTTACCTGCAGCCCAAG
CTTGCTGCACGTCTAGGGCCTCACCGGGTTTCCTTGATGAGGTACCGACATACTTATCCTGTCCCTTT
TTTTTCCACAGCTCGCGGTTGAGGACAAACTCTTC
SEQ ID NO: 22
>CBE Promoter
CTAGTTATTAATAGTAATCAATTACGGGGTCATTAGTTCATAGCCCATATATGGAGTTCCGCGTTACA
TAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAA
CTGCCCACTTGGCAGTACATCAAGTGTATCATATGCCAAGTACGCCCCCTATTGACGTCAATGACGGT
AAATGGCCCGCCTGGCATTATGCCCAGTACATGACCTTATGGGACTTTCCTACTTGGCAGTACATCTA
CGTATTAGTCATCGCTATTACCATGGTCGAGGTGAGCCCCACGTTCTGCTTCACTCTCCCCATCTCCC
CCCCCTCCCCACCCCCAATTTTGTATTTATTTATTTTTTAATTATTTTGTGCAGCGATGGGGGCGGGG
GGGGGGGGGGGGCGCGCGCCAGGCGGGGCGGGGCGGGGCGAGGGGCGGGGCGGGGCGAGGCGGAGAGG
TGCGGCGGCAGCCAATCAGAGCGGCGCGCTCCGAAAGTTTCCTTTTATGGCGAGGCGGCGGCGGCGGC
GGCCCTATAAAAAGCGAAGCGCGCGGCGGGCGCTTTTTCGCAACGGGTTTGCCGCCAGAACACAGGTA
AGTGCCGTGTGTGGTTCCCGCGGGCCTGGCCTCTTTACGGGTTATGGCCCTTGCGTGCCTTGAATTAC
TTCCACGCCCCTGGCTGCAGTACGTGATTCTTGATCCCGAGCTTCGGGTTGGAAGTGGGTGGGAGAGT
TCGAGGCCTTGCGCTTAAGGAGCCCCTTCGCCTCGTGCTTGAGTTGAGGCCTGGCTTGGGCGCTGGGG
CCGCCGCGTGCGAATCTGGTGGCACCTTCGCGCCTGTCTCGCTGCTTTCGATAAGTCTCTAGCCATTT
AAAATTTTTGATGACCTGCTGCGACGCTTTTTTTCTGGCAAGATAGTCTTGTAAATGCGGGCCAAGAT
CTGCACACTGGTATTTCGGTTTTTGGGGCCGCGGGCGGCGACGGGGCCCGTGCGTCCCAGCGCACATG
TTCGGCGAGGCGGGGCCTGCGAGCGCGGCCACCGAGAATCGGACGGGGGTAGTCTCAAGCTGGCCGGC
CTGCTCTGGTGCCTGGCCTCGCGCCGCCGTGTATCGCCCCGCCCTGGGCGGCAAGGCTGGCCCGGTCG
GCACCAGTTGCGTGAGCGGAAAGATGGCCGCTTCCCGGCCCTGCTGCAGGGAGCTCAAAATGGAGGAC
GCGGCGCTCGGGAGAGCGGGCGGGTGAGTCACCCACACAAAGGAAAAGGGCCTTTCCGTCCTCAGCCG
TCGCTTCATGTGACTCCACGGAGTACCGGGCGCCGTCCAGGCACCTCGATTAGTTCTCGAGCTTTTGG
AGTACGTCGTCTTTAGGTTGGGGGGAGGGGTTTTATGCGATGGAGTTTCCCCACACTGAGTGGGTGGA
GACTGAAGTTAGGCCAGCTTGGCACTTGATGTAATTCTCCTTGGAATTTGCCCTTTTTGAGTTTGGAT
CTTGGTTCATTCTCAAGCCTCAGACAGTGGTTCAAAGTTTTTTTCTTCCATTTCAGGTGTCGTGA
SEQ ID NO: 23
>Methyl-CpG Binding Domain (MBD) of MeCP2
TCTGCCTCCCCCAAACAGCGGCGCTCCATCATCCGTGACCGGGGACCCATGTATGATGACCCCACCCT
GCCTGAAGGCTGGACACGGAAGCTTAAGCAAAGGAAATCTGGCCGCTCTGCTGGGAAGTATGATGTGT
ATTTGATCAATCCCCAGGGAAAAGCCTTTCGCTCTAAAGTGGAGTTGATTGCGTACTTCGAAAAGGTA
GGCGACACATCCCTGGAC
SEQ ID NO: 24
>NCoR/SMRT Interaction Domain (NID) of MeCP2
AAGAAAGCCGTGAAGGAGTCTTCTATCCGATCTGTGCAGGAGACCGTACTCCCCATCAAGAAGCGCAA
GACCCGG
SEQ ID NO: 25
RTT254/NGN-401
CCTGCAGGCAGCTGCGCGCTCGCTCGCTCACTGAGGCCGCCCGGGCAAAGCCCGGGCGTCGGGCGACC
TTTGGTCGCCCGGCCTCAGTGAGCGAGCGAGCGCGCAGAGAGGGAGTGGCCAACTCCATCACTAGGGG
TTCCTGCGGCCGCACGCGTACTAGTTATTAATAGTAATCAATTACGGGGTCATTAGTTCATAGCCCAT
ATATGGAGTTCCGCGTTACATAACTTACGGTAAATGGCCCGCCTGGCTGACCGCCCAACGACCCCCGC
CCATTGACGTCAATAATGACGTATGTTCCCATAGTAACGCCAATAGGGACTTTCCATTGACGTCAATG
GGTGGAGTATTTACGGTAAACTGCCCACTTGGCAGTACATCAAGTGTATCATATGCCAAGTACGCCCC
CTATTGACGTCAATGACGGTAAATGGCCCGCCTGGCATTATGCCCAGTACATGACCTTATGGGACTTT
CCTACTTGGCAGTACATCTACGTATTAGTCATCGCTATTACCATGGTCGAGGTGAGCCCCACGTTCTG
CTTCACTCTCCCCATCTCCCCCCCCTCCCCACCCCCAATTTTGTATTTATTTATTTTTTAATTATTTT
GTGCAGCGATGGGGGCGGGGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTCCGAAAGTTTCCTTTTAT
GGCGAGGCGGCGGCGGCGGCGGCCCTATAAAAAGCGAAGCGCGCGGCGGGCGGATTCTTCTGACACAA
CAGTCTCGAACTTAAGCTGCAGAAGTTGGTCGTGAGGCACTGGGCAGGTGTCCACTCCCAGTTCAATT
ACAGCTCTTAAGGCTAGAGTACTCGGAAACCCGTCGGCCTCCGAACGGTAAGAGCCTAGCATGTAGAA
CTGGTTACCTGCAGCCCAAGCTTGCTGCACGTCTAGGGCGGACTGTTTGAATGAGGCTTCAGTACTTT
ACAGAATCGTTGCCTGCACATCTTGGAAACACTTGCTGGGATTACTTCGACTTCTTAACCCAACAGAA
GGCTCGAGAAGGTATATTGCTGTTGACAGTGAGCGAAACGATATGGGCTGAATACAATAGTGAAGCCA
CAGATGTATTGTATTCAGCCCATATCGTTGTGCCTACTGCCTCGGACTTCAAGGGGCTAGAATTCGAG
CAATTATCTTGTTTACTAAAACTGAATACCTTGCTATCTCTTTGATACATTTTTACAAAGCTGAATTA
AAATGGTATAAATTAAATCACTTTCAGCTCACCGGGTTTCCTTGATGAGGTACCGACATACTTATCCT
GTCCCTTTTTTTTCCACAGCTCGCGGTTGAGGACAAACTCTTCACCGGTCGCCACCATGGCCGCCGCC
GCCGCCGCCGCGCCGAGCGGAGGAGGAGGAGGAGGCGAGGAGGAGAGACTGGAAGAAAAGTCAGAAGA
CCAGGACCTCCAGGGCCTCAAGGACAAACCCCTCAAGTTTAAAAAGGTGAAGAAAGATAAGAAAGAAG
AGAAAGAGGGCAAGCATGAGCCCGTGCAGCCATCAGCCCACCACTCTGCTGAGCCCGCAGAGGCAGGC
AAAGCAGAGACATCAGAAGGGTCAGGCTCCGCCCCGGCTGTGCCGGAAGCTTCTGCCTCCCCCAAACA
GCGGCGCTCCATCATCCGTGACCGGGGACCCATGTATGATGACCCCACCCTGCCTGAAGGCTGGACAC
GGAAGCTTAAGCAAAGGAAATCTGGCCGCTCTGCTGGGAAGTATGATGTGTATTTGATCAATCCCCAG
GGAAAAGCCTTTCGCTCTAAAGTGGAGTTGATTGCGTACTTCGAAAAGGTAGGCGACACATCCCTGGA
CCCTAATGATTTTGACTTCACGGTAACTGGGAGAGGGAGCCCCTCCCGGCGAGAGCAGAAACCACCTA
AGAAGCCCAAATCTCCCAAAGCTCCAGGAACTGGCAGAGGCCGGGGACGCCCCAAAGGGAGCGGCACC
ACGAGACCCAAGGCGGCCACGTCAGAGGGTGTGCAGGTGAAAAGGGTCCTGGAGAAAAGTCCTGGGAA
GCTCCTTGTCAAGATGCCTTTTCAAACTTCGCCAGGGGGCAAGGCTGAGGGGGGTGGGGCCACCACAT
CCACCCAGGTCATGGTGATCAAACGCCCCGGCAGGAAGCGAAAAGCTGAGGCCGACCCTCAGGCCATT
CCCAAGAAACGGGGCCGAAAGCCGGGGAGTGTGGTGGCAGCCGCTGCCGCCGAGGCCAAAAAGAAAGC
CGTGAAGGAGTCTTCTATCCGATCTGTGCAGGAGACCGTACTCCCCATCAAGAAGCGCAAGACCCGGG
AGACGGTCAGCATCGAGGTCAAGGAAGTGGTGAAGCCCCTGCTGGTGTCCACCCTCGGTGAGAAGAGC
GGGAAAGGACTGAAGACCTGTAAGAGCCCTGGGCGGAAAAGCAAGGAGAGCAGCCCCAAGGGGCGCAG
CAGCAGCGCCTCCTCACCCCCCAAGAAGGAGCACCACCACCATCACCACCACTCAGAGTCCCCAAAGG
CCCCCGTGCCACTGCTCCCACCCCTGCCCCCACCTCCACCTGAGCCCGAGAGCTCCGAGGACCCCACC
AGCCCCCCTGAGCCCCAGGACTTGAGCAGCAGCGTCTGCAAAGAGGAGAAGATGCCCAGAGGAGGCTC
ACTGGAGAGCGACGGCTGCCCCAAGGAGCCAGCTAAGACTCAGCCCGCGGTTGCCACCGCCGCCACGG
CCGCAGAAAAGTACAAACACCGAGGGGAGGGAGAGCGCAAAGACATTGTTTCATCCTCCATGCCAAGG
CCAAACAGAGAGGAGCCTGTGGACAGCCGGACGCCCGTGACCGAGAGAGTTAGCTGAGGATCCTAATG
CTGCTATGAAACGATATGGGCTGAATACAAATCACAGGCTATGAAACGATATGGGCTGAATACAAATC
ACAGGCTATGAAACGATATGGGCTGAATACAAATCACAGGGTAGCTAGCATAATCAACCTCTGGATTA
CAAAATTTGTGAAAGATTGACTGGTATTCTTAACTATGTTGCTCCTTTTACGCTATGTGGATACGCTG
CTTTAATGCCTTTGTATCATGCTATTGCTTCCCGTATGGCTTTCATTTTCTCCTCCTTGTATAAATCC
TGGTTAGTTCTTGCCACGGCGGAACTCATCGCCGCCTGCCTTGCCCGCTGCTGGACAGGGGCTCGGCT
GTTGGGCACTGACAATTCCGTGGTGAGCTCACTTGTTTATTGCAGCTTATAATGGTTACAAATAAAGC
AATAGCATCACAAATTTCACAAATAAAGCATTTTTTTCACTGCATTCTAGTTGTGGTTTGTCCAAACT
CATCAATGTATCTTATCATGTCTGTTCCGGACACGTGCGGACCGAGCGGCCGCAGGAACCCCTAGTGA
TGGAGTTGGCCACTCCCTCTCTGCGCGCTCGCTCGCTCACTGAGGCCGGGCGACCAAAGGTCGCCCGA
CGCCCGGGCTTTGCCCGGGCGGCCTCAGTGAGCGAGCGAGCGCGCAGCTGCCTGCAGG

### EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

1. A polynucleotide comprising from 5′ to 3′:
a promoter;
at least one non-mammalian or synthetic miRNA expressed within an intron;
a protein translation initiation site (Kozak sequence);
a human MECP2 coding sequence comprising of at least 90% identity to SEQ ID NO:7, optionally comprising SEQ ID NO: 7, or a functionally active fragment thereof;
at least one 3′ stability element;
at least three miRNA binding sites for the non-mammalian or synthetic miRNA;
optionally the miRNA binding site comprises from one to six mismatches, optionally a single mismatch;
and a polyadenylation signal.

2. A polynucleotide comprising from 5′ to 3′:
a promoter;
at least one non-mammalian or synthetic miRNA expressed within an intron;
a protein translation initiation site (Kozak sequence);
a codon optimized or wildtype human MECP2 coding sequence;
at least one 3′ stability element;
three miRNA binding sites for the non-mammalian or synthetic miRNA; optionally wherein the miRNA binding site comprises a single mismatch;
and a polyadenylation signal.

3. The polynucleotide of claim 1, wherein the polynucleotide comprises one non-mammalian or synthetic miRNA expressed within an intron.

4. The polynucleotide of claim 1, wherein the non-mammalian or synthetic miRNA comprises SEQ ID NO: 4.

5. The polynucleotide of claim 1, wherein the human MECP2 coding sequence or codon optimized coding sequence comprises a nucleotide sequence having at least 90% identity to SEQ ID NO:7, optionally at least 95% identity, at least 97% identity, at least 99% identity to SEQ ID NO: 7 or 100% identity to SEQ ID NO: 7, or a functionally active fragment thereof.

6. The polynucleotide of claim 2, wherein the MECP2 sequence is a codon optimized human MECP2 sequence.

7. The polynucleotide of claim 1, wherein the protein translation initiation site is a Kozak sequence comprising SEQ ID NO: 13.

8. The polynucleotide of claim 1, wherein the promoter comprises CBM or CBE (SEQ ID NO:21 or 22).

9. The polynucleotide of claim 1, wherein the CBM promoter comprises a nucleotide sequence having at least 90% identity to SEQ ID NO:21.

10. The polynucleotide of claim 1, wherein the CBE promoter comprises a nucleotide sequence having at least 90% identity to SEQ ID NO:22.

11. The polynucleotide of claim 1, wherein the at least one 3′ stability element is WPRE.

12. The polynucleotide of claim 1, wherein the polynucleotide comprises three miRNA binding sites for the non-mammalian or synthetic miRNA.

13. The polynucleotide of claim 1, wherein the miRNA binding sites comprise SEQ ID NO: 8.

14. The polynucleotide of claim 1, wherein the miRNA binding site comprises one mismatch.

15. The polynucleotide of claim 1, wherein the polyadenylation signal is a simian vacuolating virus 40 polyadenylation signal (SV40 pA).

16. The polynucleotide of claim 15, wherein the SV40 pA signal comprises the nucleotide sequence of SEQ ID NO:12.

17. The polynucleotide of claim 1, wherein the polynucleotide comprises: a CBM promoter, one non-mammalian or synthetic miRNA expressed within an intron, a wild-type human MECP2 coding sequence with an optimized Kozak sequence, a WPRE stability element, three miRNA binding sites for the non-mammalian or synthetic miRNA, and an SV40 pA signal.

18. The polynucleotide of claim 2, wherein the polynucleotide comprises: a CBM promoter, one non-mammalian or synthetic miRNA expressed within an intron, a codon optimized human MECP2 coding sequence with an optimized Kozak sequence, a WPRE stability element, three miRNA binding sites for the non-mammalian or synthetic miRNA, and an SV40 pA signal.

19. The polynucleotide of claim 17, wherein the polynucleotide construct comprises SEQ ID NO: 25 (RTT254/NGN-401).

20. The polynucleotide of claim 1, wherein the polynucleotide further comprises at least one adeno-associated virus (AAV) inverted terminal repeat (ITR).

21. The polynucleotide of claim 20, wherein the polynucleotide comprises two AAV ITRs.

22. A vector comprising the polynucleotide of claim 1.

23. The vector of claim 22, wherein the vector is a viral vector.

24. The viral vector of claim 23, wherein the vector is an adeno-associated virus (AAV) vector.

25. The AAV vector of claim 24, wherein the AAV vector is an AAV9 vector.

26. A recombinant adeno-associated virus (rAAV), comprising the polynucleotide or vector of claim 22.

27. The rAAV of claim 26, wherein the rAAV is AAV9.

28. A virion comprising the rAAV of claim 26.

29. A transformed cell comprising the polynucleotide, the vector, the rAAV, or the virion of claim 26.

30. A pharmaceutical composition comprising the polynucleotide, the vector, the rAAV, or the virion of claim 29, and further comprising a pharmaceutically acceptable carrier.

31. A method of treating a MECP2-associated disorder in a subject, the method comprising administering to the subject an effective amount of a polynucleotide comprising from 5′ to 3′:
a promoter;
at least one non-mammalian or synthetic miRNA expressed within an intron;
a protein translation initiation site (Kozak sequence);
a human MECP2 coding sequence comprising of at least 90% identity to SEQ ID NO:7, optionally comprising SEQ ID NO: 7, or a functionally active fragment thereof;
at least one 3′ stability element;
at least three miRNA binding sites for the non-mammalian or synthetic miRNA; optionally the miRNA binding site comprises from one to six mismatches, optionally a single mismatch; and a polyadenylation signal.

32. The method of claim 31, wherein the subject exhibits improvement in one or more symptoms associated with a MECP2-associated disorder.

33. The method of claim 31, wherein the subject is dosed with 1.0×10¹⁵ vg comprising NGN-401 (SEQ ID NO:25), delivered via a 10 mL ICV injection at 1.0×10¹⁴ vg/mL.

34. The method of claim 33, wherein the effective dose is 8.3×10¹¹ vg/g brain.

35. The method of claim 31, wherein the subject is substantially free of MECP2 overexpression toxicity.

36. An effective amount of a polynucleotide as claimed in claim 1 for use as a medicament.

37. An effective amount of a polynucleotide as claimed in claim 1 for use in treating a MECP2-associated disorder in a subject.

38. The polynucleotide for use as claimed in claim 37, wherein the subject exhibits improvement in one or more symptoms associated with a MECP2-associated disorder.

39. The polynucleotide for use as claimed in claim 37, wherein the subject is dosed with 1.0×10¹⁵ vg comprising NGN-401 (SEQ ID NO:25), delivered via a 10 mL ICV injection at 1.0×10¹⁴ vg/mL.

40. The polynucleotide for use as claimed in claim 39, wherein the effective dose is 8.3×10¹¹ vg/g brain.

41. The polynucleotide for use as claimed in claim 37, wherein the subject is substantially free of MECP2 overexpression toxicity.
